(19) United States
(12) Patent Application Publication
(10) Pub. No.: US 2026/0002104 A1
(43) Pub. Date: Jan. 1, 2026

(54) DEVICES AND METHODS FOR CELL AGGREGATION

(71) Applicant: Satellite Biosciences, Inc., Cambridge, MA (US)

(72) Inventors: Thomas Jay LOWERY, JR., Sudbury, MA (US); Christopher Garrison WILSON, Auburndale, MA (US); William SCHOLL, Cambridge, MA (US); Marcus LEHMANN, Boston, MA (US); Christopher MONTALBANO, Huntington, NY (US); Greg MONTALBANO, Huntington, NY (US); Andrew MARTIN, Glen Cove, NY (US)

(21) Appl. No.: 18/869,182

(22) PCT Filed: May 26, 2023
(86) PCT No.: PCT/US2023/067536

§ 371 Date: Nov. 25, 2024

Related U.S. Application Data
(60) Provisional application No. 63/346,582, filed on May 27, 2022.

(51) Int. Cl. C12M 1/32; C12M 1/00; C12M 1/12

(52) U.S. Cl. CPC C12M 23/12; C12M 23/40; C12M 25/06; C12M 29/00

(57) ABSTRACT

Featured is a device for aggregating populations of cells that includes one or more plates that include a microwell film composite. The microwell film composite includes a first layer with a plurality of microwells and a second layer that includes a permeable body that includes a plurality of pores. The device further includes one or more ports for fluid input and/or output. Also featured are methods of making aggregates including a first population of cells (e.g., parenchymal cells (e.g., hepatocytes)) and a second population of cells (e.g., stromal cells).

### FIELD OF THE INVENTION

The present invention relates generally to devices for aggregating populations of cells.

### BACKGROUND OF THE INVENTION

Many diseases result from damage, malfunction, or loss of a single organ or tissue type. While certain strategies such as organ transplants can be effective, the demand for replacement organs is great. Tissue therapeutics, including the development of engineered tissue constructs (e.g., cell-based implants), are among the most promising multidisciplinary approaches to fulfill this demand. However, despite significant advances in the fields of cell biology, microfluidics, and engineering, to date, conventional approaches have failed to re-create functional tissues at a scale necessary to impart therapeutic efficacy. Formation of cell aggregates to generate tissues is critical as a first step towards creating useful tissues. However, mimicking biological conditions to achieve such conditions remains challenging. Accordingly, new devices and methods for forming cell aggregates are needed.

### SUMMARY OF THE INVENTION

In one aspect, the invention features a device that includes one or more plates that include a microwell film composite. The microwell film composite includes a first layer with a plurality of microwells and a second layer that includes a permeable body that includes a plurality of pores. The device further includes one or more ports for fluid input and/or output. The device may further include a macroscopic manifold that is connected to the microwell film composite and is configured to direct fluid flow to the microwell film composite. The macroscopic manifold may include one or more of the ports for fluid input and/or output. In some embodiments, the plurality of pores is formed by non-woven non-linear pathways. The pores may be, e.g., cylindrical. In some embodiments, the pores each include a through hole.

In some embodiments, the device further includes one or more gaskets that form a seal between the macroscopic manifold and the microwell film composite.

In some embodiments, the macroscopic manifold further includes one or more seal ports in fluid connection with the microwell film composite. The seal ports are configured to direct fluid flow and/or pressure to or from the microwell film composite. In some embodiments, the macroscopic manifold further includes one or more foil seals attached to the seal ports. The macroscopic manifold may further include one or more seal punches positioned in the one or more seal ports. The seal punches are configured to break a corresponding foil seal, e.g., to actuate fluid communication through the seal port. In some embodiments, the macroscopic manifold includes two or more (e.g., three, four, five, or more) seal ports and two or more (e.g., three, four, five, or more) seal punches. In some embodiments, the macroscopic manifold further includes one or more seal bellows that connect the one or more seal punches to the one or more seal ports.

In some embodiments, the macroscopic manifold further includes a sealed outlet in fluid connection with the one or more seal ports, wherein the sealed outlet is configured to direct fluid flow from the microwells to the one or more seal ports.

In some embodiments, the macroscopic manifold further includes a sealed inlet in fluid connection with the one or more seal ports, wherein the sealed inlet is configured to direct fluid flow from the microwells to the one or more seal ports.

In some embodiments, the device further includes a cam bar connected to the one or more seal punches. The device may include an actuator (e.g., a button or lever) connected to the cam bar and configured to engage the one or more seal punches. The cam bar may be connected to the macroscopic manifold.

In some embodiments, the device further includes a collection outlet in fluid communication with the first layer that includes the plurality of microwells. The collection outlet may be used to collect material (e.g., cells or aggregates) in the microwells that do not pass through the permeable layer.

In some embodiments, the plate has a surface area of from 100 mm² to 100,000 mm². For example, the plate may have a surface area of 100 to 1,000 mm², e.g., 100 mm², 200 mm², 300 mm², 400 mm², 500 mm², 600 mm², 700 mm², 800 mm², 900 mm², or 1,000 mm², e.g., from 1,000 mm² to 10,000 mm², e.g., 2,000 mm², 3,000 mm², 4,000 mm², 5,000 mm², 6,000 mm², 7,000 mm², 8,000 mm², 9,000 mm², or 10,000 mm², e.g., from 10,000 mm² to 100,000 mm², e.g., 20,000 mm², 30,000 mm², 40,000 mm², 50,000 mm², 60,000 mm², 70,000 mm², 80,000 mm², 90,000 mm², or 100,000 mm².

In some embodiments, the plate has a density of microwells of from 1 well/mm² to 10⁶ wells/mm². For example, the plate may have a density of microwells of from 1 well/mm² to 10 well/mm², e.g., 2 well/mm², 3 well/mm², 4 well/mm², 5 well/mm², 6 well/mm², 7 well/mm², 8 well/mm², 9 well/mm², or 10 well/mm², e.g., from 10 well/mm² to 100 well/mm², e.g., 20 well/mm², 30 well/mm², 40 well/mm², 50 well/mm², 60 well/mm², 70 well/mm², 80 well/mm², 90 well/mm², or 100 well/mm², e.g., from 100 well/mm² to 1,000 well/mm², e.g., 200 well/mm², 300 well/mm², 400 well/mm², 500 well/mm², 600 well/mm², 700 well/mm², 800 well/mm², 900 well/mm², or 1,000 well/mm², e.g., from 1,000 well/mm² to 10,000 well/mm², e.g., 2,000 well/mm², 3,000 well/mm², 4,000 well/mm², 5,000 well/mm², 6,000 well/mm², 7,000 well/mm², 8,000 well/mm², 9,000 well/mm², or 10,000 well/mm², e.g., from 10,000 well/mm² to 100,000 well/mm², e.g., 20,000 well/mm², 30,000 well/mm², 40,000 well/mm², 50,000 well/mm², 60,000 well/mm², 70,000 well/mm², 80,000 well/mm², 90,000 well/mm², or 100,000 well/mm², e.g., from 100,000 well/mm² to 106 wells/mm², e.g., 200,000 wells/mm², 300,000 wells/mm², 400,000 wells/mm², 500,000 wells/mm², 600,000 wells/mm², 700,000 wells/mm², 800,000 wells/mm², 900,000 wells/mm², or 10⁶ wells/mm².

In some embodiments, each microwell has a diameter of from 1 μm to 1 mm. For example, each microwell may have a diameter of from 1 μm to 10 μm, e.g., 2 μm, 3 μm, 4 μm, 5 μm, 6 μm, 7 μm, 8 μm, 9 μm, or 10 μm, e.g., from 10 μm to 100 μm, e.g., 20 μm, 30 μm, 40 μm, 50 μm, 60 μm, 70 μm, 80 μm, 90 μm, or 100 μm, e.g., from 100 μm to 1 mm, e.g., 200 μm, 300 μm, 400 μm, 500 μm, 600 μm, 700 μm, 800 μm, 900 μm, or 1 mm.

In some embodiments, the permeable body includes a polymer. The polymer may include, for example, polycarbonate, polyester, polystyrene, polytetrafluoroethylene (PTFE), collagen-coated PTFE, polyethylene terephthalate (PET), polysulfone (PES), nylon, or cellulose acetate. In some embodiments, the permeable body may include stainless steel.

In some embodiments, the microwells include a stainless material or a polymer. For example, in some embodiments, the microwells include a polymer, such as polystyrene, PET, polycarbonate, polypropylene, cellulose acetate, PES, or fluorinated ethylene propylene (FEP).

In some embodiments, each pore has a diameter of from 0.1 μm to 15 μm. For example, each pore may have a diameter of from 0.1 μm to 1 μm, e.g., 0.2 μm, 0.3 μm, 0.4 μm, 0.5 μm, 0.6 μm, 0.7 μm, 0.8 μm, 0.9 μm, or 1 μm, e.g., from 1 μm to 15 μm, e.g., 2 μm, 3 μm, 4 μm, 5 μm, 6 μm, 7 μm, 8 μm, 9 μm, 10 μm, 11 μm, 12 μm, 13 μm, 14 μm, or 15 μm. In some embodiments, each pore has a diameter of from 0.1 μm to 3 μm. In some embodiments, each pore has a diameter of 5 μm.

In some embodiments, the permeable body has a density of pores across the permeable body of from 1×10⁴ pores/cm² to 1×10⁹ pores/cm². For example, the permeable body may have a density of pores across the permeable body of from 10⁴ pores/cm² to 10⁵ pores/cm², e.g., 20,000 pores/cm², 30,000 pores/cm², 40,000 pores/cm², 50,000 pores/cm², 60,000 pores/cm², 70,000 pores/cm², 80,000 pores/cm², 90,000 pores/cm², or 10⁵ pores/cm², e.g., from 10⁵ pores/cm² to 10⁶ pores/cm², e.g., 2×10⁵ pores/cm², 3×10⁵ pores/cm², 4×10⁵ pores/cm², 5×10⁵ pores/cm², 6×10⁵ pores/cm², 7×10⁵ pores/cm², 8×10⁵ pores/cm², 9×10⁵ pores/cm², or 10⁶ pores/cm², e.g., from 10⁶ pores/cm² to 10⁷ pores/cm², e.g., 2×10⁷ pores/cm², 3×10⁷ pores/cm², 4×10⁷ pores/cm², 5×10⁷ pores/cm², 6×10⁷ pores/cm², 7×10⁷ pores/cm², 8×10⁷ pores/cm², 9×10⁷ pores/cm², or 10⁸ pores/cm², e.g., from 10⁸ pores/cm² to 10⁹ pores/cm², e.g., 2×10⁸ pores/cm², 3×10⁸ pores/cm², 4×10⁸ pores/cm², 5×10⁸ pores/cm², 6×10⁸ pores/cm², 7×10⁸ pores/cm², 8×10⁸ pores/cm², 9×10⁸ pores/cm², or 10⁹ pores/cm².

In some embodiments, the permeable body is a porous filter.

In some embodiments, the layer that includes the microwells further includes a passivation coating. The passivation coating may include a non-covalent coating and/or a covalent coating. For example, in some embodiments, the non-covalent coating includes a block co-polymer, polyethylene glycol (PEG), streptavidin, albumin/biotin, phospholipid surfactants, hyaluronic acid, or poly-lysine-based adherence. In some embodiments, the covalent coating includes a covalent attachment (e.g., carboxylic acid/amine bonds, disulfide bonds, n-hydroxysuccinimide (NHS) esters, NHS, maleimide, cycloadditions, epoxy, amine, carboxy, aldehyde, p-phenylene diisothiocyanate (PDITC), maleimide, thiol, poly-l-lysine, streptavidin, or neutravidin).

In some embodiments, the microwells are conical, spherical, cylindrical, pyramidal, or chaliced shaped.

In some embodiments, the device includes a plate cover.

In some embodiments, the one or more plates further include an upper manifold. The upper manifold may be in fluid connection with the macroscopic manifold and is configured to direct fluid flow and/or fluid distribution to or from the macroscopic manifold to the microwell film composite. In some embodiments, the device further includes a retaining ring positioned between the upper manifold and the microwell film composite. The retaining ring may be configured to position the upper manifold from 10 μm to 100 mm (e.g., from 1 μm to 10 μm, e.g., 2 μm, 3 μm, 4 μm, 5 μm, 6 μm, 7 μm, 8 μm, 9 μm, or 10 μm, e.g., from 10 μm to 100 μm, e.g., 20 μm, 30 μm, 40 μm, 50 μm, 60 μm, 70 μm, 80 μm, 90 μm, or 100 μm, e.g., from 100 μm to 1 mm, e.g., 200 μm, 300 μm, 400 μm, 500 μm, 600 μm, 700 μm, 800 μm, 900 μm, or 1 mm, e.g., from 1 mm to 10 mm, e.g., 2 mm, 3 mm, 4 mm, 5 mm, 6 mm, 7 mm, 8 mm, 9 mm, or 10 mm, e.g., from 10 mm to 100 mm, e.g., 20 mm, 30 mm, 40 mm, 50 mm, 60 mm, 70 mm, 80 mm, 90 mm, or 100 mm) from the microwell film composite.

In some embodiments, the upper manifold includes an upper surface and a lower surface, and the one or more plates further include a plate cover positioned on the upper surface of the upper manifold.

In some embodiments, the first layer and the second layer are components of a laminate. The laminate may further include a third layer that includes a perforation film. The third layer may be adjoined to the layer that includes the permeable body and is configured to direct fluid pressure to the microwell film composite. The perforation film may include a polymer, such as polystyrene. In some embodiments, the microwells include polystyrene, and the permeable body includes PTFE or collagen-coated PTFE, and the perforation film includes polystyrene.

In some embodiments, the one or more plates further include a film superstructure that is connected to the microwell film composite and is configured to provide structural integrity to the layers of the microwell film composite. The film superstructure may include one or more gaps across the film superstructure, and the one or more gaps allow fluid to flow through the layer that includes a permeable body. In some embodiments, the one or more gaps are configured to be a distance of 10 μm to 100 mm (e.g., from 1 μm to 10 μm, e.g., 2 μm, 3 μm, 4 μm, 5 μm, 6 μm, 7 μm, 8 μm, 9 μm, or 10 μm, e.g., from 10 μm to 100 μm, e.g., 20 μm, 30 μm, 40 μm, 50 μm, 60 μm, 70 μm, 80 μm, 90 μm, or 100 μm, e.g., from 100 μm to 1 mm, e.g., 200 μm, 300 μm, 400 μm, 500 μm, 600 μm, 700 μm, 800 μm, 900 μm, or 1 mm, e.g., from 1 mm to 10 mm, e.g., 2 mm, 3 mm, 4 mm, 5 mm, 6 mm, 7 mm, 8 mm, 9 mm, or 10 mm, e.g., from 10 mm to 100 mm, e.g., 20 mm, 30 mm, 40 mm, 50 mm, 60 mm, 70 mm, 80 mm, 90 mm, or 100 mm) apart.

In some embodiments, the one or more plates further include a membrane film standoff that is connected to the film superstructure to provide structural integrity to the microwell film composite.

In some embodiments, the one or more plates further include a basin that is connected to the film superstructure to provide a receptacle for fluid that flows through the layer including the permeable body.

The basin may have a height of 10 μm to 100 mm (e.g., from 1 μm to 10 μm, e.g., 2 μm, 3 μm, 4 μm, 5 μm, 6 μm, 7 μm, 8 μm, 9 μm, or 10 μm, e.g., from 10 μm to 100 μm, e.g., 20 μm, 30 μm, 40 μm, 50 μm, 60 μm, 70 μm, 80 μm, 90 μm, or 100 μm, e.g., from 100 μm to 1 mm, e.g., 200 μm, 300 μm, 400 μm, 500 μm, 600 μm, 700 μm, 800 μm, 900 μm, or 1 mm, e.g., from 1 mm to 10 mm, e.g., 2 mm, 3 mm, 4 mm, 5 mm, 6 mm, 7 mm, 8 mm, 9 mm, or 10 mm, e.g., from 10 mm to 100 mm, e.g., 20 mm, 30 mm, 40 mm, 50 mm, 60 mm, 70 mm, 80 mm, 90 mm, or 100 mm).

In some embodiments, the device includes a plurality of plates. For example, the device may include 5, 6, 7, 8, 9, 10, 11, 12, 13, 14, 15, 16, 17, 18, 19, 20, or more plates.

In another aspect, the invention features a method of using a device as described herein. The methods employ using a device for aggregating a population of cells, such as a population of stromal cells and/or a population of parenchymal cells. The method of aggregation may include aggregating a first population of cells and a second population of cells. The method includes the steps of (a) providing a device as described herein and a medium (e.g., perfusion medium) that includes a population of cells (e.g., a first population of cells, e.g., stromal cells, and a second population of cells, e.g., parenchymal cells); and (b) introducing the medium (e.g., perfusion medium) that includes the population of cells (e.g., the first population of cells, e.g., stromal cells, and the second population of cells, e.g., parenchymal cells) into the device through one of the ports to direct the medium (e.g., perfusion medium) that includes the cells into the microwells.

The aggregates may include, for example, two or more populations of cells (e.g., two, three, four, five, six, seven, eight, nine, ten, or more populations of cells).

In some embodiments the first population of cells and/or the second population of cells are induced pluripotent (iPSC)-derived cells, engineered cells, primary cells, embryonic stem cells (ESC)-derived cells, or transdifferentiated cells.

The introduction of the first population of cells and the second population of cells can be sequential, e.g., the first population of cells may be introduced, e.g., in a first medium, and the second population of cells may be introduced, e.g., in a second medium. Similarly, the second population of cells may be introduced, e.g., in a second medium, and the first population of cells may be introduced, e.g., in a first medium. Alternatively, the first population of cells and the second population of cells may be introduced in the same medium.

The introduction of stromal cells and parenchymal cells can be sequential, e.g., the stromal cells may be introduced, e.g., in a first medium, and the parenchymal cells may be introduced, e.g., in a second medium. Similarly, the parenchymal cells may be introduced, e.g., in a first medium, and the stromal cells may be introduced, e.g., in a second medium. Alternatively, the stromal cells and the parenchymal cells may be introduced in the same medium.

In some embodiments, the device includes a macroscopic manifold connected to the microwell film composite, and step (b) includes applying a positive pressure to the macroscopic manifold to direct fluid flow to the microwell film composite.

In some embodiments, step (b) includes shaking or centrifuging the device.

In some embodiments, each pore allows flow of the aggregation medium, and each pore prevents flow of population of cells, e.g., a population of stromal or parenchymal cells.

In some embodiments, step (b) further includes introducing an aggregation medium through the port.

In some embodiments, step (b) includes applying a pressure to the macroscopic manifold to direct the aggregation medium into the microwells.

In some embodiments, the method further includes the step of (c) culturing the first population of cells and the second population of cells in the microwells under conditions (e.g., perfusion conditions) such that aggregates of the first population of cells and the second population of cells form in the microwells.

In some embodiments, step (c) further includes culturing the first population of cells and the second population of cells in the microwells under conditions (e.g., perfusion conditions) in the aggregation medium such that aggregates of the first population of cells and the second population of cells form in the microwells.

In some embodiments, the method further includes washing or removing the aggregation medium.

In some embodiments, the method further includes the step of (d) releasing the aggregates from the microwells, thereby producing a population of aggregates including the first population of cells and the second population of cells. Step (d) may include, for example, applying a negative pressure to the macroscopic manifold to release the aggregates. Alternatively, this step may include decanting or pipetting the aggregates to release them. The method may further include collecting the population of aggregates. In some embodiments, the device includes a collection outlet in fluid communication with the first layer that includes the plurality of microwells, and the method includes collecting the population of aggregates as it passes through the collection outlet.

The first population of cells may include, for example, stromal cells. The second population of cells may include, for example, parenchymal cells.

In some embodiments, the parenchymal cells include hepatocytes, pancreatic exocrine cells, myocytes, pancreatic endocrine cells, neurons, enterocytes, adipocytes, splenic cells, kidney cells, biliary cells, Kupffer cells, stellate cells, cardiac muscle cells, alveolar cells, bronchiolar cells, club cells, urothelial cells, mucous cells, parietal cells, chief cells, G cells, goblet cells, enteroendocrine cells, Paneth cells, M cells, tuft cells, glial cells, gall bladder cells, keratinocytes, melanocytes, Merkel cells, Langerhans cells, osteocytes, osteoclasts, esophageal cells, photoreceptor cells, or corneal epithelial cells. In some embodiments, the hepatocytes are primary human hepatocytes (PHH), iPSC-derived hepatocytes, or ESC-derived hepatocytes. In some embodiments, the parenchymal cells are pancreatic cells (e.g., alpha, beta, gamma, delta, epsilon cells, or any combination thereof). In some embodiments, the primary cells include primary cells expanded in vitro.

In some embodiments, the engineered cells are engineered to express or secrete a protein (e.g., an antibody, a cytokine, an enzyme, a coagulation factor, or a hormone). In some embodiments, the protein is an endogenous human protein or an engineered protein.

In some embodiments, the first and/or second population of cells includes endocrine, exocrine, paracrine, heterocrine, autocrine, or juxtacrine cells.

In some embodiments, the first and/or second population of cells includes leading cells, adrenal cortical cells, pituitary cells, thyrocytes, granulosa cells, mammary gland epithelial cells, thymocytes, thymic epithelial cells, hypothalamus cells, skeletal muscle cells, smooth muscle cells, and/or neuronal cells.

In some embodiments, the pituitary cells include thyrotropic pituitary cells, lactotropic pituitary cells, corticotropic pituitary cells, somatotropic pituitary cells, and/or gonadotropic pituitary cells. In some embodiments, the neuronal cells include dopaminergic cells.

In some embodiments, the first and/or second population of cells includes parenchymal cells (e.g., hepatocytes, pancreatic exocrine cells, myocytes, pancreatic endocrine cells, neurons, enterocytes, adipocytes, splenic cells, kidney cells, biliary cells, Kupffer cells, stellate cells, cardiac muscle cells, alveolar cells, bronchiolar cells, club cells, urothelial cells, mucous cells, parietal cells, chief cells, G cells, goblet cells, enteroendocrine cells, Paneth cells, M cells, tuft cells, glial cells, gall bladder cells, keratinocytes, melanocytes, Merkel cells, Langerhans cells, osteocytes, osteoclasts, esophageal cells, photoreceptor cells, and corneal epithelial cells). In some embodiments, the parenchymal cells are pancreatic cells (e.g., alpha, beta, gamma, delta, epsilon cells, or any combination thereof). In some embodiments, the parenchymal cells include beta cells.

In some embodiments, the first and/or second population of cells are engineered cells, primary cells, or transdifferentiated cells.

In some embodiments, the method includes encapsulating two or more populations of cells (e.g., two, three, four, five, six, seven, eight, nine, ten, or more populations of cells).

In some embodiments, the parenchymal cells include primary human hepatocytes (PHH). In some embodiments, the population of PHH includes from 2.5×10⁴ to 1.8×10¹¹ (e.g., from 3×10⁴ to 1.8×10¹¹, 4×10⁴ to 1.8×10¹¹, 5×10⁴ to 1.8×10¹¹, 1×10⁵ to 1.8×10¹¹, 4×10⁵ to 1.8×10¹¹, 5×10⁵ to 1.8×10¹¹, 6×10⁵ to 1.8×10¹¹, 7×10⁵ to 1.8×10¹¹, 8×10⁵ to 1.8×10¹¹, 9×10⁵ to 1.8×10¹¹, 1×10⁶ to 1.8×10¹¹, 2×10⁶ to 1.8×10¹¹, 3×10⁶ to 1.8×10¹¹, 4×10⁶ to 1.8×10¹¹, 5×10⁶ to 1.8×10¹¹, 6×10⁶ to 1.8×10¹¹, 7×10⁶ to 1.8×10¹¹, 8×10⁶ to 1.8×10¹¹, 9×10⁶ to 1.8×10¹¹, 1×10⁷ to 1.8×10¹¹, 2×10⁷ to 1.8×10¹¹, 1.8×10⁷ to 1.8×10¹¹, 4×10⁷ to 1.8×10¹¹, 5×10⁷ to 1.8×10¹¹, 6×10⁷ to 1.8×10¹¹, 7×10⁷ to 1.8×10¹¹, 8×10⁷ to 1.8×10¹¹, 9×10⁷ to 1.8×10¹¹, 1×10⁸ to 1.8×10¹¹, 2×10⁸ to 1.8×10¹¹, 3×10⁸ to 1.8×10¹¹, 4×10⁸ to 1.8×10¹¹, 5×10⁸ to 1.8×10¹¹, 6×10⁸ to 1.8×10¹¹, 7×10⁸ to 1.8×10¹¹, 8×10⁸ to 1.8×10¹¹, 9×10⁸ to 1.8×10¹¹, 1×10⁹ to 1.8×10¹¹, 2×10⁹ to 1.8×10¹¹, 3×10⁹ to 1.8×10¹¹, 4×10⁹ to 1.8×10¹¹, 5×10⁹ to 1.8×10¹¹, 6×10⁹ to 1.8×10¹¹, 7×10⁹ to 1.8×10¹¹, 8×10⁹ to 1.8×10¹¹, 9×10⁹ to 1.8×10¹¹, 1×10¹⁰ to 1.8×10¹¹, 2×10¹⁰ to 1.8×10¹¹, 3×10¹⁰ to 1.8×10¹¹, 4×10¹⁰ to 1.8×10¹¹, 5×10¹⁰ to 1.8×10¹¹, 6×10¹⁰ to 1.8×10¹¹, 7×10¹⁰ to 1.8×10¹¹, 8×10¹⁰ to 1.8×10¹¹, 9×10¹⁰ to 1.8×10¹¹, or 1×10¹¹ to 1.8×10¹¹) PHH.

In some embodiments, a ratio of hepatocytes to stromal cells is from 1:10 to 4:1 (e.g., 1:10 to 4:1, 1:10 to 3:1, 1:10 to 2:1, 1:10 to 1:1, 1:9 to 4:1, 1:9 to 3:1, 1:9 to 2:1, 1:9 to 1:1, 1:8 to 4:1, 1:8 to 3:1, 1:8 to 2:1, 1:8 to 1:1, 1:7 to 4:1, 1:7 to 3:1, 1:7 to 2:1, 1:7 to 1:1, 1:6 to 4:1, 1:6 to 3:1, 1:6 to 2:1, 1:6 to 1:1, 1:5 to 4:1, 1:5 to 3:1, 1:5 to 2:1, 1:5 to 1:1, 1:4 to 4:1, 1:4 to 3:1, 1:4 to 2:1, 1:4 to 1:1, 1:3 to 4:1, 1:3 to 3:1, 1:3 to 2:1, 1:3 to 1:1, 1:2 to 4:1, 1:2 to 3:1, 1:2 to 2:1, 1:2 to 1:1, 1:1 to 4:1, 1:1 to 3:1, 1:1 to 2:1, or 1:0 to 1:1)

In some embodiments, the stromal cells are fibroblasts, endothelial cells, or pericytes. In some embodiments, the fibroblasts are normal human dermal fibroblasts or neonatal foreskin fibroblasts. In some embodiments, the fibroblasts are normal human dermal fibroblasts.

In some embodiments, the population of stromal cells includes from 6×10³ to 1.8×10¹² e.g., from 1 to 1.8×10¹², from 10 to 1.8×10¹², from 100 to 1.8×10¹², from 1×10³ to 1.8×10¹², from 2×10³ to 1.8×10¹², from 3×10³ to 1.8×10¹², from 4×10³ to 1.8×10¹², from 5×10³ to 1.8×10¹², from 6×10³ to 1.8×10¹², from 7×10³ to 1.8×10¹², from 8×10³ to 1.8×10¹², from 9×10³ to 1.8×10¹², from 1×10⁴ to 1.8×10¹², from 2×10⁴ to 1.8×10¹², from 3×10⁴ to 1.8×10¹², from 4×10⁴ to 1.8×10¹², from 5×10⁴ to 1.8×10¹², from 6×10⁴ to 1.8×10¹², from 7×10⁴ to 1.8×10¹², from 8×10⁴ to 1.8×10¹², from 9×10⁴ to 1.8×10¹², from 1×10⁵ to 1.8×10¹², from 2×10⁵ to 1.8×10¹², from 3×10⁵ to 1.8×10¹², from 4×10⁵ to 1.8×10¹², from 5×10⁵ to 1.8×10¹², from 6×10⁵ to 1.8×10¹², from 7×10⁵ to 1.8×10¹², from 8×10⁵ to 1.8×10¹², from 9×10⁵ to 1.8×10¹², from 1×10⁶ to 1.8×10¹², from 2×10⁶ to 1.8×10¹², 3×10⁶ to 1.8×10¹², 4×10⁶ to 1.8×10¹², 5×10⁶ to 1.8×10¹², 6×10⁶ to 1.8×10¹², 7×10⁶ to 1.8×10¹², 8×10⁶ to 1.8×10¹², 9×10⁶ to 1.8×10¹², from 1×10⁷ to 1.8×10¹², from 2×10⁷ to 1.8×10¹², from 18×10⁷ to 1.8×10¹², from 4×10⁷ to 1.8×10¹², from 5×10⁷ to 1.8×10¹², from 6×10⁷ to 1.8×10¹², from 7×10⁷ to 1.8×10¹², from 8×10⁷ to 1.8×10¹², from 9×10⁷ to 1.8×10¹², from 1×10⁸ to 1.8×10¹², from 2×10⁸ to 1.8×10¹², from 3×10⁸ to 1.8×10¹², from 4×10⁸ to 1.8×10¹², from 5×10⁸ to 1.8×10¹², from 6×10⁸ to 1.8×10¹², from 7×10⁸ to 1.8×10¹², from 8×10⁸ to 1.8×10¹², from 9×10⁸ to 1.8×10¹², from 1×10⁹ to 1.8×10¹², from 2×10⁹ to 1.8×10¹², from 3×10⁹ to 1.8×10¹², from 4×10⁹ to 1.8×10¹², from 5×10⁹ to 1.8×10¹², from 6×10⁹ to 1.8×10¹², from 7×10⁹ to 1.8×10¹², from 8×10⁹ to 1.8×10¹², from 9×10⁹ to 1.8×10¹², from 1×10¹⁰ to 1.8×10¹², from 2×10¹⁰ to 1.8×10¹², from 3×10¹⁰ to 1.8×10¹², from 4×10¹⁰ to 1.8×10¹², from 5×10¹⁰ to 1.8×10¹², from 6×10¹⁰ to 1.8×10¹², from 7×10¹⁰ to 1.8×10¹², from 8×10¹⁰ to 1.8×10¹², from 9×10¹⁰ to 1.8×10¹², from 1×10¹¹ to 1.8×10¹², from 2×10¹¹ to 1.8×10¹², from 3×10¹¹ to 1.8×10¹², from 4×10¹¹ to 1.8×10¹², from 5×10¹¹ to 1.8×10¹², from 6×10¹¹ to 1.8×10¹², from 7×10¹¹ to 1.8×10¹², from 8×10¹¹ to 1.8×10¹², from 9×10¹¹ to 1.8×10¹², or from 1×10¹² to 1.8×10¹²) stromal cells.

In some embodiments, the population of parenchymal cells and the population of stromal cells are provided in a ratio of parenchymal cells to stromal cells of from 1:10 to 4:1 (e.g., 1:10 to 4:1, 1:10 to 3:1, 1:10 to 2:1, 1:10 to 1:1, 1:9 to 4:1, 1:9 to 3:1, 1:9 to 2:1, 1:9 to 1:1, 1:8 to 4:1, 1:8 to 3:1, 1:8 to 2:1, 1:8 to 1:1, 1:7 to 4:1, 1:7 to 3:1, 1:7 to 2:1, 1:7 to 1:1, 1:6 to 4:1, 1:6 to 3:1, 1:6 to 2:1, 1:6 to 1:1, 1:5 to 4:1, 1:5 to 3:1, 1:5 to 2:1, 1:5 to 1:1, 1:4 to 4:1, 1:4 to 3:1, 1:4 to 2:1, 1:4 to 1:1, 1:3 to 4:1, 1:3 to 3:1, 1:3 to 2:1, 1:3 to 1:1, 1:2 to 4:1, 1:2 to 3:1, 1:2 to 2:1, 1:2 to 1:1, 1:1 to 4:1, 1:1 to 3:1, 1:1 to 2:1, or 1:0 to 1:1).

In some embodiments, the one or more plates further include an upper manifold. The upper manifold is in fluid connection with the macroscopic manifold and directs fluid flow and/or fluid distribution to or from the macroscopic manifold to the microwell film composite.

In some embodiments, the macroscopic manifold further includes one or more seal ports in fluid connection with the microwell film composite. The one or more seal ports direct fluid flow and/or pressure to or from the microwell film composite.

In some embodiments, the macroscopic manifold further includes one or more foil seals attached to the seal ports. The macroscopic manifold may further include one or more seal punches positioned in the one or more seal ports, and the seal punches break the one or more foil seals. In some embodiments, the device further includes a cam bar connected to the one or more seal punches, and the method includes actuating the cam bar to engage the one or more seal punches to break the one or more foil seals.

In another aspect, the invention features a population of aggregates including a first population of cells and a second population of cells produced by a method as described herein. In some embodiments, the first population of cells includes stromal cells (e.g., fibroblasts) and the second population of cells includes parenchymal cells (e.g., hepatocytes).

### BRIEF DESCRIPTION OF THE DRAWINGS

FIG. 1 is a schematic drawing of an exemplary plate with a microwell film composite. Both the 3D rectangular and square structures represent the plate, while the flat lattice represents the microwell film composite that includes multiple layers, including a layer with microwells and a layer with a permeable body, such as a filter.

FIG. 2 is a schematic drawing of a plate (1) with a microwell film composite (2).

FIG. 3 is a schematic drawing showing a close-up view of a microwell film composite (2).

FIG. 4 is a schematic drawing showing a perspective view of a plate with a macroscopic manifold (3).

FIG. 5 is a schematic drawing showing a perspective view of a plate with a macroscopic manifold (3).

FIG. 6 is a schematic drawing showing a perspective cross-sectional view of a plate with a macroscopic manifold (3).

FIG. 7 is a schematic drawing showing a perspective cross-sectional view of a plate.

FIG. 8 is a schematic drawing showing an exploded view of a device as described herein.

FIG. 9 is a schematic drawing showing a cross sectional view of the microwells (4). The cells sit in the microwells but cannot penetrate the permeable body (5).

FIG. 10 is a schematic drawing showing a cross sectional view of the microwells (4) and the direction of flow. The cells sit in the microwells but cannot penetrate the permeable body (5).

FIG. 11 is a schematic drawing showing a cross sectional view of the microwells (4) with cell aggregates being removed by reversing the direction of flow.

FIG. 12 is a schematic drawing showing a cross sectional view of the microwells (4) and a permeable body (5) that includes pores (6).

FIG. 13 is a schematic drawing showing a cross sectional view of the microwells (4) and a permeable body (5) that includes pores (6) disposed under each microwell.

FIG. 14 is a schematic drawing showing a cross sectional view of a microwell (4) with a chalice (left) or conical (right) shape.

FIG. 15 is a schematic drawing showing a cross sectional view of a plate with upper manifold (9), retaining ring (10), and a film superstructure (27).

FIG. 16 is a schematic drawing showing a close-up cross-sectional view of a plate with retaining ring (10) and a film superstructure (27). The layers of the permeable body (5) are in a laminate that includes a perforation film (13) and membrane film standoff (15).

FIG. 17 is a schematic drawing showing a cross-sectional view of a plate with the direction of flow coming from the upper manifold (9).

FIG. 18 is a schematic drawing showing a close-up cross-sectional view of a plate as in FIG. 17.

FIG. 19 is a schematic drawing showing a cross-sectional of a plate with upper manifold (9). The cells sit in the microwells but cannot penetrate the permeable body (5).

FIG. 20 is a schematic drawing showing a close-up cross-sectional view of a plate as in FIG. 19.

FIG. 21 is a schematic drawing showing a close-up cross-sectional view of a plate as in FIG. 19. The cells settle into microwells (4).

FIG. 22 is a schematic drawing showing a close-up cross-sectional view of a plate as in FIG. 19. The cells form aggregates in microwells (4).

FIG. 23 is a schematic drawing showing a close-up cross-sectional view of a plate as in FIG. 19. The aggregates in microwells (4) are released by reversing the direction of flow.

FIG. 24 is a schematic drawing showing a close-up cross-sectional view of a plate as in FIG. 19. The aggregates in microwells (4) are released by reversing the direction of flow and flow through upper manifold (9).

FIG. 25 is a schematic drawing of macroscopic manifold (3) with a vector map illustrating fluid flow.

FIG. 26 is a schematic drawing of macroscopic manifold (3) with a vector map illustrating fluid flow.

FIG. 27 is a schematic drawing of a macroscopic manifold (3) that includes a seal punch (20) positioned in a seal port.

FIG. 28 is a schematic drawing of a macroscopic manifold (3) that includes a seal punch (20) positioned in a seal port. The seal punch (20) has been actuated to open the seal port.

FIG. 29 is a schematic drawing showing a cross-sectional view of FIG. 28. The seal punch (20) has not yet been actuated to open the seal port (19) by breaking foil seal (18).

FIG. 30 is a schematic drawing showing macroscopic manifold (3) with an actuator (24) that engages seal punch (20) via cam bar (23).

FIG. 31 is a schematic drawing showing macroscopic manifold (3) with an actuator (24) that engages seal punch (20) via cam bar (23).

FIG. 32 is a schematic drawing showing close-up view of cam bar (23) engaging seal punch (20).

FIG. 33 is a schematic drawing showing a device with a plurality of plates (1) and a plurality of macroscopic manifolds (3).

FIG. 34 is a schematic drawing of a device with a plate with a microwell film composite (2) without a macroscopic manifold. The microwells can be filled, e.g., manually, by shaking, or by centrifugation.

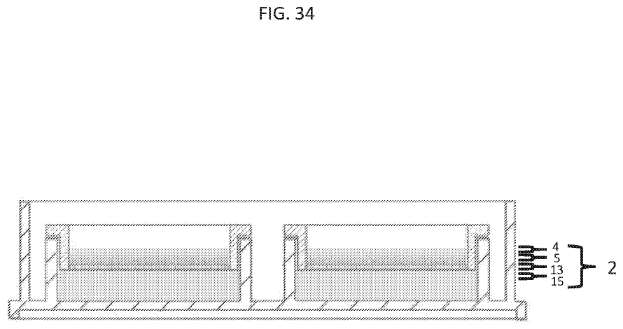

FIG. 35 is a schematic drawing of a device with a plate with a microwell film composite (2) without a macroscopic manifold and plate cover (11).

FIG. 36 is a schematic drawing of a device with a plate, inlet ports (7A and 7B) and outlet ports (7C and 7D). The media flows through left top inlet port (7A) and out of bottom outlet port (7D) under the microwell film composite (2).

FIG. 37 is a schematic drawing of a device with a plate, inlet ports (7A and 7B) and outlet ports (7C and 7D). The cells flow through right top inlet port (7B) and the media flows out through bottom outlet port (7D) under the microwell film composite (2).

FIG. 38 is a schematic drawing of a device with a plate, inlet ports (7A and 7B) and outlet ports (7C and 7D). The media flows through top left inlet port (7A) and out bottom outlet port (7D) under the microwell film composite (2). The cells settle into the microwells.

FIG. 39 is a schematic drawing of a device with a plate, inlet ports (7A and 7B) and outlet ports (7C and 7D). The portion of the plate above the microwell film composite (2) has been drained through top right outlet port (7C) such that the liquid level remains just under top right outlet port (7C). The bottom right outlet port (7D) is closed, such that fluid beneath the microwell film composite (2) cannot flow out.

FIG. 40 is a schematic drawing of a device with a plate in which the cells are incubated after the top inlet ports (7A and 7B) as shown in FIGS. 36-38 are removed. The device includes plate cover (11).

FIG. 41 is a schematic drawing of a device with a plate in which the aggregates are released by reversing flow through bottom right outlet port (7D).

FIG. 42 is a schematic drawing showing a layer with cylindrical wells.

FIG. 43 is a schematic drawing showing a layer with pyramidal wells.

FIG. 44 is a schematic drawing showing a layer with tapered side square wells.

FIG. 45 is a schematic drawing showing a layer with cylindrical wells.

FIG. 46 is a schematic drawing showing a layer with tapered side square wells.

FIG. 47 is a schematic drawing showing a layer with microwells.

FIG. 48 is a schematic drawing showing a layer with microwells.

FIG. 49 is a schematic drawing showing a layer with microwells.

FIG. 50 is a schematic drawing showing a layer with microwells.

FIG. 51 is a schematic drawing showing a layer with microwells.

FIG. 52 is a schematic drawing showing a layer with microwells.

FIG. 53 is a schematic drawing showing a microwell film composite with a first layer with a plurality of microwells and a permeable body disposed below the microwells.

FIG. 54 is a schematic drawing showing a microwell film composite with a first layer with a plurality of microwells, a permeable body disposed below the microwell, and a bottom film.

FIG. 55 is a schematic drawing showing a device as described herein with exemplary dimensions (mm).

FIGS. 56A and 56B are photographs showing a perspective view (FIG. 56A) and a top-down view (FIG. 56B) of a device as described herein.

FIG. 57 is a photograph showing different microwell sizes.

FIG. 58 is a set of photographs showing different components that can be disposed in a well.

FIG. 59 is a schematic drawing showing an angled inlet manifold.

FIG. 60 is a schematic drawing showing a dual inlet manifold.

FIG. 61 is a schematic drawing showing a loop inlet manifold.

FIG. 62 is a schematic drawing showing a bifurcation manifold.

FIG. 63 is a schematic drawing showing a macroscopic manifold.

FIG. 64 is a schematic drawing showing a radial manifold.

FIG. 65 is a schematic drawing showing a radial channel manifold.

### DEFINITIONS

Cells can be from established cell lines or they can be primary cells, where “primary cells,” “primary cell lines,” and “primary cultures” are used interchangeably herein to refer to cells and cells cultures that have been derived from an individual (e.g., a human individual) and allowed to grow in vitro for a limited number of passages, e.g., splitting, of the culture. For example, primary cultures can be cultures that have been passaged 0 times, 1 time, 2 times, 4 times, 5 times, 10 times, or 15 times, but not enough times go through the crisis stage. Primary cell lines can be maintained for fewer than 10 passages in vitro. If the cells are primary cells, such cells can be harvested from an individual by any convenient method. For example, cells from tissues such as skin, muscle, bone marrow, spleen, liver, pancreas, lung, intestine, stomach, or other tissues are most conveniently harvested by biopsy. An appropriate solution can be used for dispersion or suspension of the harvested cells. Such solution will generally be a balanced salt solution, e.g., normal saline, phosphate-buffered saline (PBS), Hank's balanced salt solution, and the like, conveniently supplemented with fetal calf serum or other naturally occurring factors, in conjunction with an acceptable buffer at low concentration, generally from 5-25 mM. Convenient buffers include HEPES (4-(2-hydroxyethyl)-1-piperazineethanesulfonic acid), phosphate buffers, lactate buffers, and the like. The cells can be used immediately, or they can be stored, frozen, for long periods of time, being thawed and capable of being reused. In such cases, the cells will usually be frozen in 10% dimethyl sulfoxide (DMSO), 50% serum, 40% buffered medium, or some other such solution as is commonly used in the art to preserve cells at such freezing temperatures and thawed in a manner as commonly known in the art for thawing frozen cultured cells. For example, hepatocytes may be isolated by conventional methods (Berry and Friend, 1969, J. Cell Biol. 43:506-520) which can be adapted for human liver biopsy or autopsy material (e.g., to garner primary human hepatocytes).

As used herein, the term “cell type” refers to a group of cells sharing a phenotype that is statistically separable based on gene expression data. For example, cells of a common cell type may share similar structural and/or functional characteristics, such as similar gene activation patterns and antigen presentation profiles. Cells of a common cell type may include those that are isolated from a common tissue (e.g., epithelial tissue, neural tissue, connective tissue, or muscle tissue) and/or those that are isolated from a common organ, tissue system, blood vessel, or other structure and/or region in an organism.

As used herein, the term “pore” refers to an opening in a surface that allows particles below a predetermined size (e.g., smaller than the pore size) to pass through. A pore may be formed by any suitable method known in the art, such as etching (e.g., chemical etching), drilling (e.g., electron beam drilling), laser, pin, ion beam, roller pin, computer numerical control (CNC) router, or the like. Pores may be formed by any structure known in the art, such as holes, voids, or non-woven nonlinear pathways.

As used herein, the term “non-woven nonlinear pathways” refers to pathways that are not linear (e.g., curvilinear) and are defined by a fabric-like material made from short and long fibers bonded together by chemical, mechanical, heat or solvent treatment. The term is used in the textile manufacturing industry to denote fabrics, such as felt, which are neither woven nor knitted as the fibers are not intertwined.

As used herein, the term “parenchymal cells” refers to cells of, or derived from, the parenchyma of an organ or gland, e.g., a mammalian organ or gland. The parenchyma of an organ or gland is the functional tissue of the organ or gland, as distinguished from surrounding or supporting or connective tissue. As such, parenchymal cells are attributed with carrying out the particular function, or functions, of the organ or gland, often referred to in the art as “tissue-specific” function. Parenchymal cells include, but are not limited to, hepatocytes, pancreatic cells (e.g., alpha, beta, gamma, delta, and epsilon cells), myocytes, e.g., smooth muscle cells, cardiac myocytes, and the like, enterocytes, renal epithelial cells and other kidney cells, brain cell (e.g., neurons or glial cells, e.g., astrocytes), respiratory epithelial cells, stem cells, and blood cells (e.g., erythrocytes and lymphocytes), adult and embryonic stem cells, blood-brain barrier cells, adipocytes, splenocytes, osteoblasts, osteoclasts, and other parenchymal cell types known in the art. Because parenchymal cells are responsible for tissue-specific function, parenchymal cells express or secrete certain tissue specific markers.

Certain precursor cells can also be included as “parenchymal cells,” in particular, if they are committed to becoming the more differentiated cells described above, for example, liver progenitor cells, oval cells, adipocytes, osteoblasts, osteoclasts, myoblasts, stem cells (e.g., embryonic stem cells, hematopoietic stem cells, mesenchymal stem cells, or endothelial stem cells), and the like. In some embodiments, stem cells can be encapsulated and/or implanted under specified conditions such that they are induced to differentiate into a desired parenchymal cell type, for example, in the construct and/or in vivo. It is also contemplated that parenchymal cells derived from cell lines can be used in the methodologies of the invention.

### DETAILED DESCRIPTION

The present invention features devices and methods for forming cell aggregates. Formation of cell aggregates for tissue engineering is critical as a first step towards creating useful tissues for various therapeutic approaches. However, mimicking biological conditions to achieve such conditions remains challenging. In particular, many devices utilize two dimensional approaches, which may only create flat layers of tissue, as compared to the three-dimensional structures required for many tissues. Moreover, creating a device for facile manipulation of cell aggregates remains challenging due to the requirement for various cell media exchanges to ensure robust cell growth and aggregation.

The present invention solves this problem by providing a device that includes a plate with a microwell film composite. The microwell film composite includes a first layer with a plurality of microwells and a permeable body disposed below the microwells. The cells are able to be deposited into the microwells, while fluids (e.g., cell culture media) can pass through the permeable body while allowing the cells to remain in place in the microwells. The density of microwells provides improved aggregation seeding and increased surface area, e.g., relative to a flat surface. The cells can be cultured in the microwells to form aggregates. The device further includes one or more fluid ports to provide fluid communication and fluid flow throughout the device. Such fluidic access provides a mechanism for supplying cells with necessary culture medium and ultimately releasing cell aggregates from the device for collection and downstream use.

### Devices

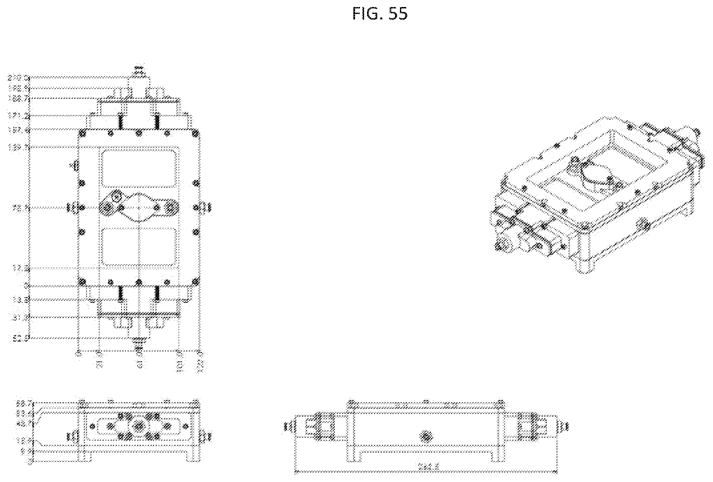

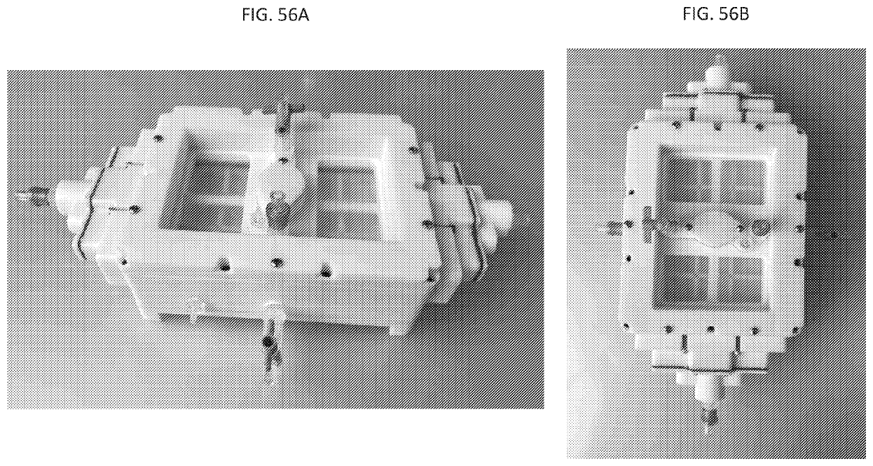

The devices described herein include a plate (1) with a microwell film composite (2) having a first layer with a plurality of microwells and a second layer that includes a permeable body (5) that includes pores (6). The pores may be formed by any suitable method known in the art, such as etching (e.g., chemical etching), drilling (e.g., electron beam drilling), laser, pin, ion beam, roller pin, computer numerical control (CNC) router, or the like. Pores may be formed by any structure known in the art, such as holes, voids, or non-woven nonlinear pathways. In some embodiments, the plurality of pores is formed by non-woven non-linear pathways. The pores may be, e.g., cylindrical. The device further includes one or more ports (7) for fluid input and/or output to provide fluid communication and fluid flow throughout the device. Such fluidic access provides a mechanism for supplying cells with necessary culture medium and ultimately releasing cell aggregates from the device for collection and downstream use. The device may further include a macroscopic manifold (3) that is connected to the microwell film composite (2) and is configured to direct fluid flow to the microwell film composite. The macroscopic manifold can apply a pressure (e.g., positive or negative pressure) on the device to manipulate the directionality of fluid flow. The device may further include one or more pumps operably connected to the device, e.g., to apply fluid flow. An exemplary device is shown in FIGS. 55 and 56.

### Plate (1)

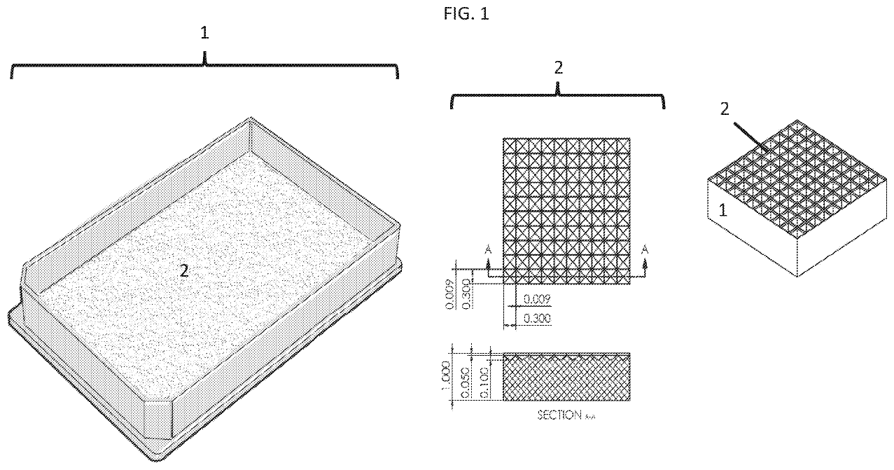

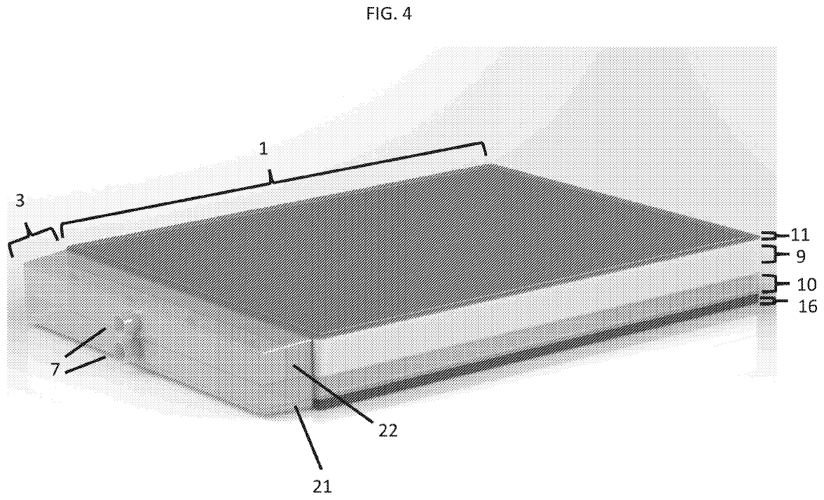

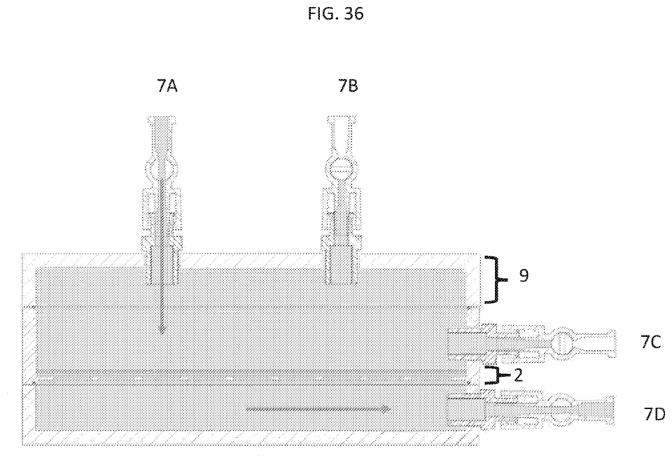

A device described herein includes a plate with a microwell film composite (2) (FIG. 1). The device may include a plurality of plates. For example, the device may include 2, 3, 4, 5, 6, 7, 8, 9, 10, 11, 12, 13, 14, 15, 16, 17, 18, 19, 20, or more plates (1), and each plate includes a corresponding microwell film composite (2). The plate may include one or more (e.g., 2, 3, 4, 5, or more) ports (7) for fluid input and/or fluid output. For example, the plate may include 1 or 2 ports for fluid input and 1 or 2 ports for fluid output (see, e.g., FIG. 4). The plate may further include a macroscopic manifold (3). The macroscopic manifold may be connected to the microwell film composite and is configured to direct fluid flow to the microwell film composite. In some embodiments, one or more of the ports (7) are located on the macroscopic manifold (3). The macroscopic manifold (3) may be positioned in any suitable location on the plate (1) to provide fluid communication with the microwell film composite (2). For example, the macroscopic manifold may be disposed on a side of the plate (1) (FIG. 4). Alternatively, the macroscopic manifold may be disposed above the plate (1) (see, e.g., manifold (9) in FIG. 36). In yet another embodiment, the macroscopic manifold may be disposed below the plate.

In some embodiments, the plate (1) has a surface area of from 100 mm² to 100,000 mm². For example, the plate (1) may have a surface area of 100 mm² to 1,000 mm², e.g., 100 mm², 200 mm², 300 mm², 400 mm², 500 mm², 600 mm², 700 mm², 800 mm², 900 mm², or 1,000 mm², e.g., from 1,000 mm² to 10,000 mm², e.g., 2,000 mm², 3,000 mm², 4,000 mm², 5,000 mm², 6,000 mm², 7,000 mm², 8,000 mm², 9,000 mm², or 10,000 mm², e.g., from 10^{,000} mm² to 100,000 mm², e.g., 20,000 mm², 30,000 mm², 40,000 mm², 50,000 mm², 60,000 mm², 70,000 mm², 80,000 mm², 90,000 mm², or 100,000 mm². In some embodiments, the plate (1) has a surface area of 200 mm² to 10⁵ mm². In some embodiments, the plate (1) has a surface area of 300 mm² to 10,000 mm². In some embodiments, the plate (1) has a surface area of 400 mm² to 1,000 mm². In some embodiments, the plate (1) has a surface area of 500 mm².

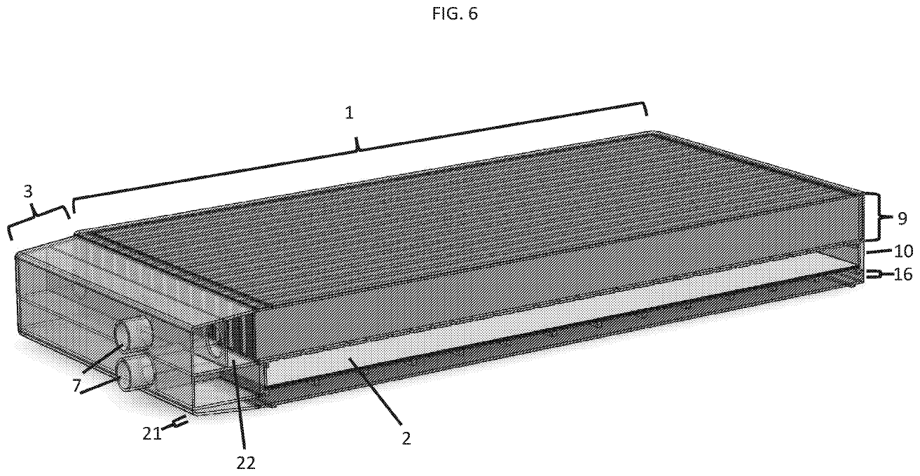

The one or more (e.g., 2, 3, 4, 5, or more) plates (1) may also include an upper manifold (9) (see, e.g., FIG. 6). Such an upper manifold may be in fluid connection with the macroscopic manifold (3) and can be configured to direct fluid flow and/or fluid distribution to or from the macroscopic manifold (3) to the microwell film composite (2).

The upper manifold may include an upper surface, and the one or more (e.g., 2, 3, 4, 5, or more) plates may further include a plate cover (11) positioned above the plate on the upper surface of the upper manifold (see, e.g., FIG. 4).

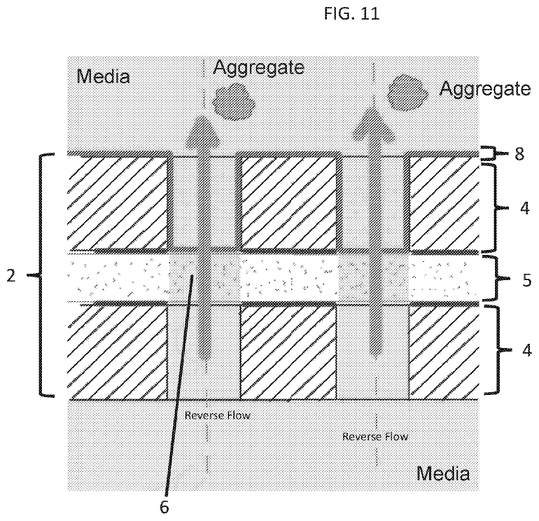

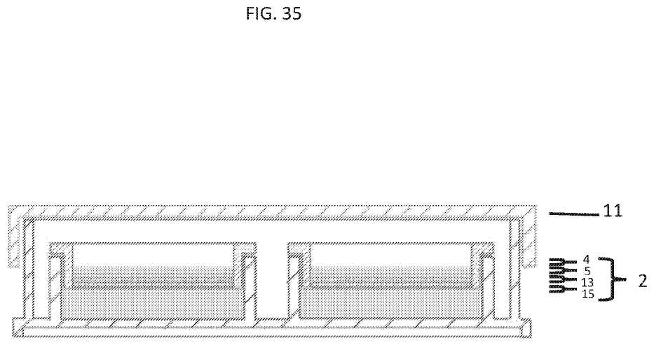

The plate may include a plate cover (11) that covers the plate (1), e.g., without a macroscopic manifold (FIGS. 11 and 35)

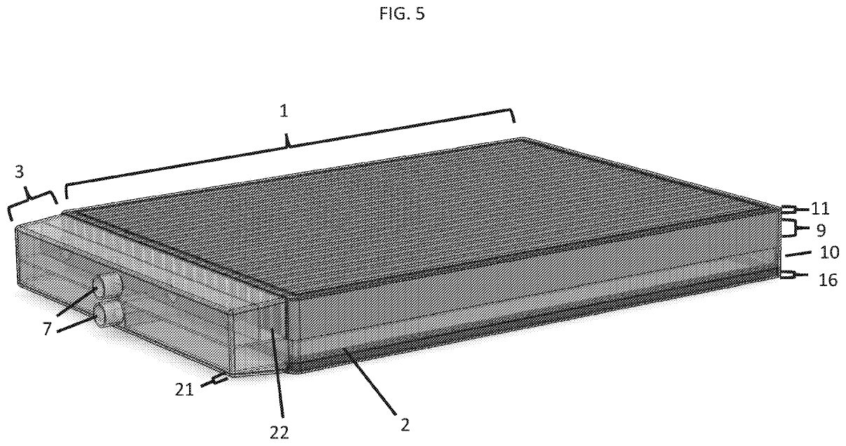

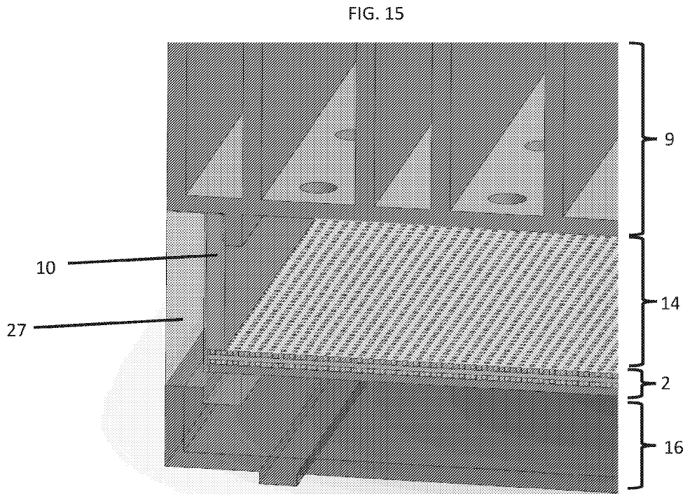

The one or more (e.g., 2, 3, 4, 5, or more) plates (1) may also include a retaining ring (10) (see, e.g., FIGS. 5 and 6). A retaining ring (10) may be positioned between the upper manifold (9) and the microwell film composite (2), such that the retaining ring is configured to position the upper manifold (9) a distance away from the microwell film composite (2) (FIG. 15). This distance may be, e.g., from 10 μm to 1×100 mm (e.g., 10 μm to 100 mm, 20 μm to 10 mm, 30 μm to 1 mm, 40 μm to 900 μm, 50 μm to 800 μm, 100 μm to 700 μm, or 500 μm to 600 μm) away from the microwell film composite (2). For example, in some embodiments, a retaining ring (10) may be positioned between the upper manifold (9) and the microwell film composite (2), such that the retaining ring is configured to position the upper manifold (9) a distance of 10 μm to 100 mm away from the microwell film composite (2). In some embodiments, a retaining ring (10) may be positioned between the upper manifold (9) and the microwell film composite (2), such that the retaining ring is configured to position the upper manifold (9) a distance of 20 μm to 10 mm away from the microwell film composite (2). In some embodiments, a retaining ring (10) may be positioned between the upper manifold (9) and the microwell film composite (2), such that the retaining ring is configured to position the upper manifold (9) a distance of 30 μm to 1 mm away from the microwell film composite (2). In some embodiments, a retaining ring (10) may be positioned between the upper manifold (9) and the microwell film composite (2), such that the retaining ring is configured to position the upper manifold (9) a distance of 40 μm to 900 away from the microwell film composite (2). In some embodiments, a retaining ring (10) may be positioned between the upper manifold (9) and the microwell film composite (2), such that the retaining ring is configured to position the upper manifold (9) a distance of 50 μm to 800 μm away from the microwell film composite (2). In some embodiments, a retaining ring (10) may be positioned between the upper manifold (9) and the microwell film composite (2), such that the retaining ring is configured to position the upper manifold (9) a distance of 100 μm to 700 μm away from the microwell film composite (2). In some embodiments, a retaining ring (10) may be positioned between the upper manifold (9) and the microwell film composite (2), such that the retaining ring is configured to position the upper manifold (9) a distance of 500 μm to 600 μm away from the microwell film composite (2).

In some embodiments, the one or more (e.g., 2, 3, 4, 5, or more) plates (1) include a film superstructure (27) (see, e.g., FIG. 15). A film superstructure (27) may be connected to the microwell film composite (2) and is configured to provide structural integrity to the layers (e.g., 4, 5, and/or 13) of the microwell film composite (2).

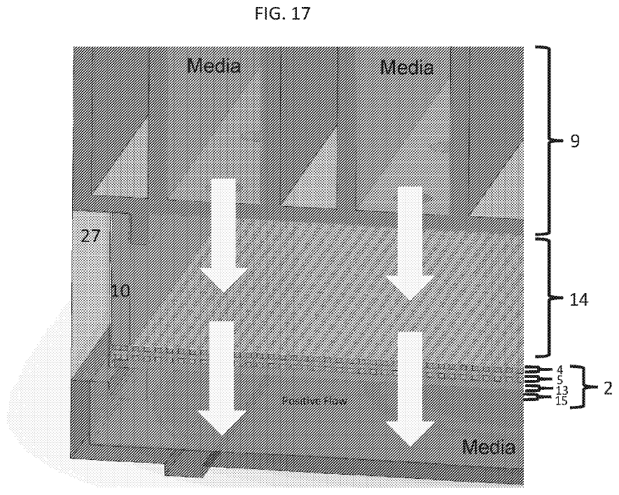

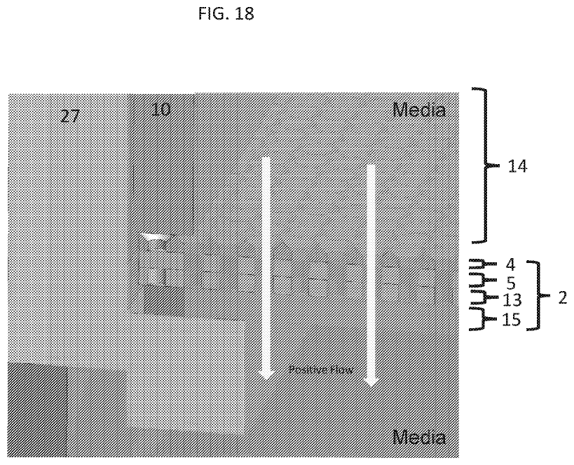

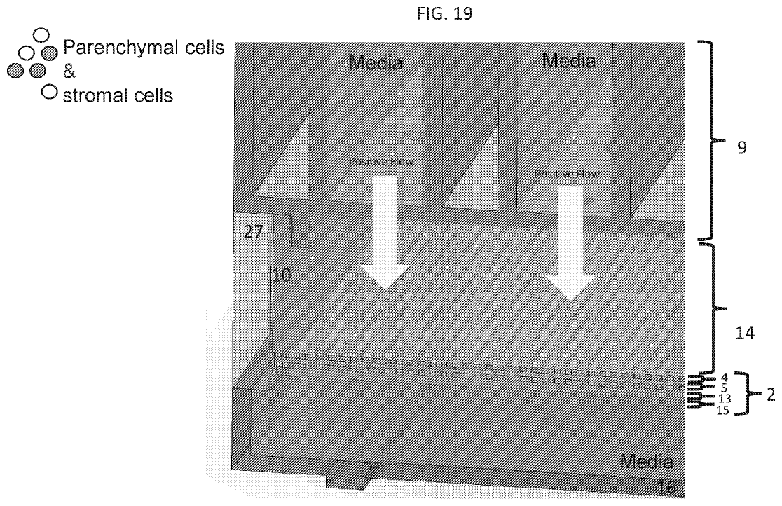

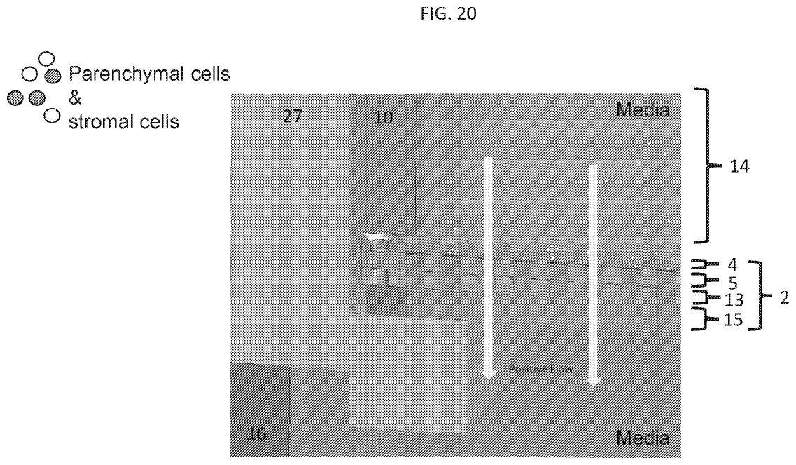

The film superstructure (27) may include one or more (e.g., 2, 3, 4, 5, or more) gaps (14) across the film superstructure (27) (FIGS. 17-20). Such gaps are included in the design to allow fluid to flow through the layer that includes a permeable body.

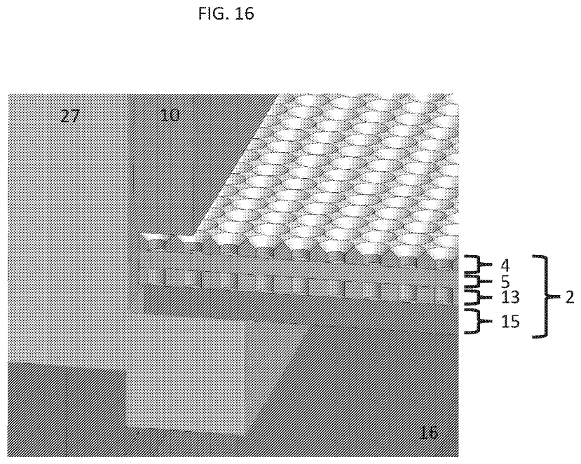

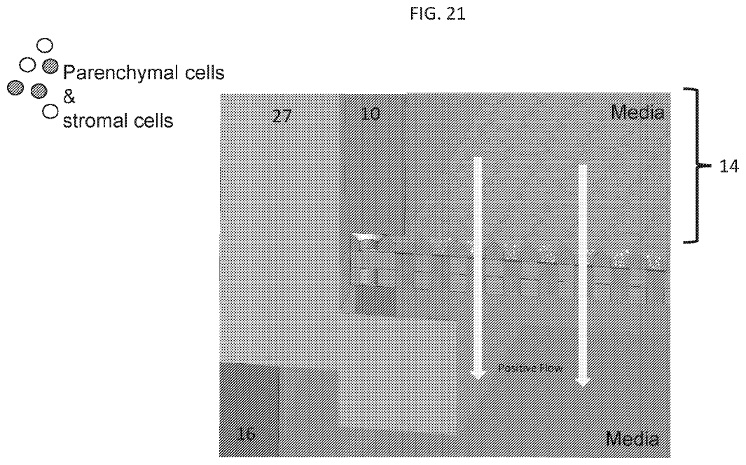

In some embodiments, the one or more (e.g., 2, 3, 4, 5, or more) plates (1) include a membrane film standoff (15) (see, e.g., FIGS. 16-24). A membrane film standoff (15) can be connected to the film superstructure (27) to provide structural integrity to the microwell film composite (2).

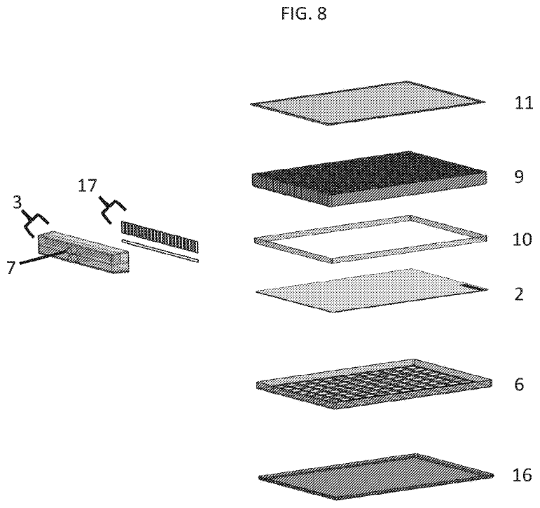

In some embodiments, the one or more (e.g., 2, 3, 4, 5, or more) plates (1) include a basin (16) (see, e.g., FIG. 8). The basin (16) can be connected to the film superstructure (27) to provide a receptacle for fluid that flows through the layer with the permeable body (5).

In some embodiments, the basin has a height of 10 μm to 100 mm (e.g., from 1 μm to 10 μm, e.g., 2 μm, 3 μm, 4 μm, 5 μm, 6 μm, 7 μm, 8 μm, 9 μm, or 10 μm, e.g., from 10 μm to 100 μm, e.g., 20 μm, 30 μm, 40 μm, 50 μm, 60 μm, 70 μm, 80 μm, 90 μm, or 100 μm, e.g., from 100 μm to 1 mm, e.g., 200 μm, 300 μm, 400 μm, 500 μm, 600 μm, 700 μm, 800 μm, 900 μm, or 1 mm, e.g., from 1 mm to 10 mm, e.g., 2 mm, 3 mm, 4 mm, 5 mm, 6 mm, 7 mm, 8 mm, 9 mm, or 10 mm, e.g., from 10 mm to 100 mm, e.g., 20 mm, 30 mm, 40 mm, 50 mm, 60 mm, 70 mm, 80 mm, 90 mm, or 100 mm).

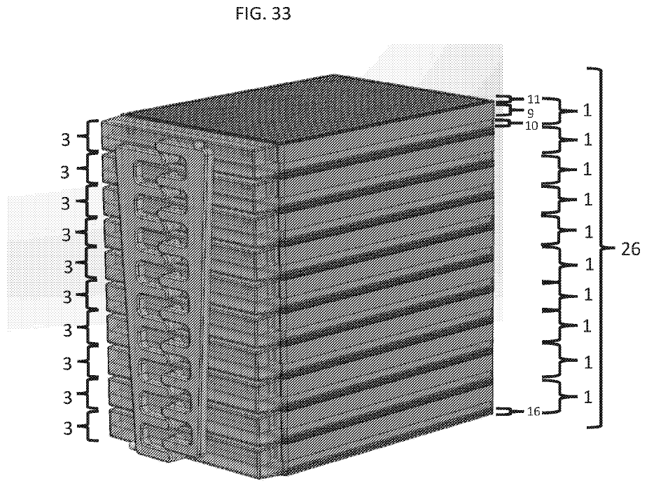

In some embodiments, the device includes more than one plates (1), such as five or more (e.g., 10, 15, 20, 30, or more) plates (26) (FIG. 33). For example, in some embodiments, the device includes ten or more plates (26). In some embodiments, the device includes 15 or more plates (26). In some embodiments, the device includes 20 or more plates (26). In some embodiments, the device includes 25 or more plates (26). In some embodiments, the device includes 30 or more plates (26).

### Microwell Film Composite (2)

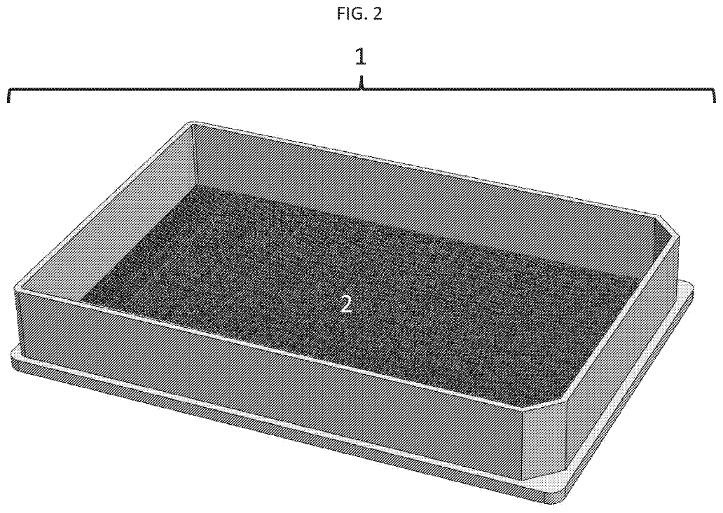

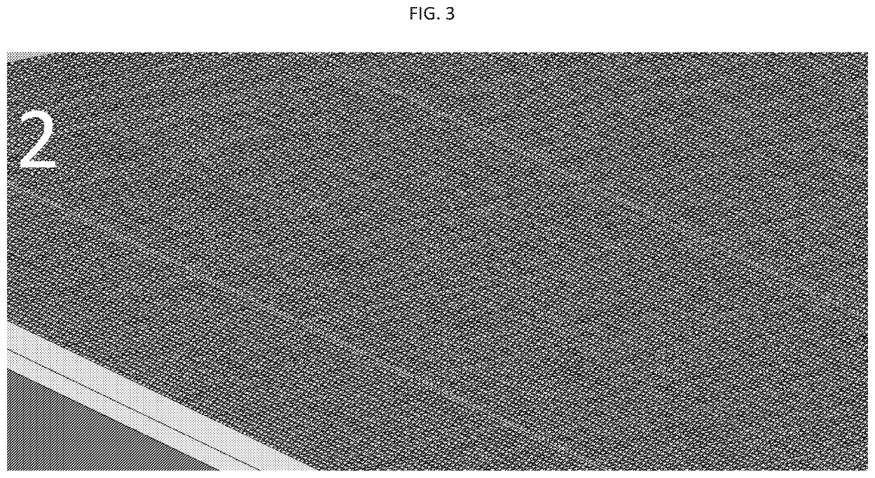

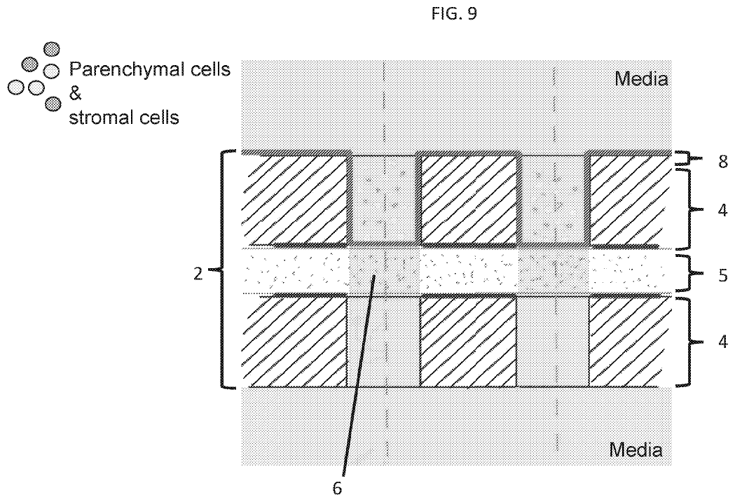

The devices described herein include a microwell film composite (2) (FIGS. 2 and 3). The microwell film composite (2) may include a first layer having a plurality of microwells (4) in which cells can be cultured and aggregated. Such a layer may also include a passivation coating (8), e.g., to prevent nonspecific binding to the surface of the microwells (4) (FIG. 9).

In some embodiments, the density of microwells (4) in the plate is from 1 well/mm² to 10⁶ wells/mm². For example, the plate may have a density of microwells of from 1 well/mm² to 10 well/mm², e.g., 2 well/mm², 3 well/mm², 4 well/mm², 5 well/mm², 6 well/mm², 7 well/mm², 8 well/mm², 9 well/mm², or 10 well/mm², e.g., from 10 well/mm² to 100 well/mm², e.g., 20 well/mm², 30 well/mm², 40 well/mm², 50 well/mm², 60 well/mm², 70 well/mm², 80 well/mm², 90 well/mm², or 100 well/mm², e.g., from 100 well/mm² to 1,000 well/mm², e.g., 200 well/mm², 300 well/mm², 400 well/mm², 500 well/mm², 600 well/mm², 700 well/mm², 800 well/mm², 900 well/mm², or 1,000 well/mm², e.g., from 1,000 well/mm² to 10,000 well/mm², e.g., 2,000 well/mm², 3,000 well/mm², 4,000 well/mm², 5,000 well/mm², 6,000 well/mm², 7,000 well/mm², 8,000 well/mm², 9,000 well/mm², or 10,000 well/mm², e.g., from 10^{,000} well/mm² to 100,000 well/mm², e.g., 20,000 well/mm², 30,000 well/mm², 40,000 well/mm², 50,000 well/mm², 60,000 well/mm², 70,000 well/mm², 80,000 well/mm², 90,000 well/mm², or 100,000 well/mm², e.g., from 100,000 well/mm² to 10⁶ wells/mm², e.g., 200,000 wells/mm², 300,000 wells/mm², 400,000 wells/mm², 500,000 wells/mm², 600,000 wells/mm², 700,000 wells/mm², 800,000 wells/mm², 900,000 wells/mm², or 10⁶ wells/mm².

In some embodiments, the microwell film composite (2) has a length and/or width that is independently, from 50 mm to 500 mm (e.g., from 50 mm to 100 mm, e.g., 50 mm, 60 mm, 70 mm, 80 mm, 90 mm, or 100 mm, e.g., from 100 mm to 500 mm, e.g., 100 mm, 150 mm, 200 mm, 250 mm, 300 mm, 350 mm, 400 mm, 450 mm, or 500 mm). In some embodiments, the microwell film composite (2) has a length and/or width that is independently 225 mm. In some embodiments, the microwell film composite (2) has a length and/or width that is independently 300 mm. In some embodiments, the microwell film composite (2) has a length of 225 mm and a width of 300 mm.

In some embodiments, the microwell film composite (2) includes from 1,000 to 500,000 microwells (4). In some embodiment, the microwell film composite (2) includes from 1,000 to 10,000 (e.g., 1,000, 2,000, 3,000, 4,000, 5,000, 6,000, 7,000, 8,000, 9,000, or 10,000), 10,000 to 100,000 (e.g., 10,000, 20,000, 30,000, 40,000, 50,000, 60,000, 70,000, 80,000, 90,000, or 100,000), or 100,000 to 500,000 (e.g., 100,000, 150,000, 200,000, 250,000, 300,000, 350,000, 400,000, 450,000, or 500,000) wells. In some embodiments, the microwell film composite (2) includes 70,000 wells. In some embodiments, the microwell film composite (2) includes 75,000 wells. In some embodiments, the microwell film composite (2) includes 86,000 wells. In some embodiments, the microwell film composite (2) includes 105,000 wells. In some embodiments, the microwell film composite (2) includes 187,000 wells. In some embodiments, the microwell film composite (2) includes 5,258 well. In some embodiments, the microwell film composite (2) includes 9,360 wells. In some embodiments, the microwell film composite (2) includes 14,962 wells. In some embodiments, the microwell film composite (2) includes 29,164 wells. In some embodiments, the microwell film composite (2) includes 37,921 wells. In some embodiments, the microwell film composite (2) includes 44,077 wells. In some embodiments, the microwell film composite (2) includes 53,061 wells. In some embodiments, the microwell film composite (2) includes 67,500 wells. In some embodiments, the microwell film composite (2) includes 107,896 wells. In some embodiments, the microwell film composite (2) includes 109,091 wells. In some embodiments, the microwell film composite (2) includes 120,000 wells. In some embodiments, the microwell film composite (2) includes 210,318 wells. In some embodiments, the microwell film composite (2) includes 317,864 wells. In some embodiments, the microwell film composite (2) includes 382,653 wells. In some embodiments, the microwell film composite (2) includes 786,713 wells. In some embodiments, the microwell film composite (2) includes 865,385 wells.

In some embodiments, each microwell (4) is from 1 μm to 100 mm (e.g., 10 μm to 100 mm, 20 μm to 10 mm, 30 μm to 1 mm, 40 μm to 900 μm, 50 μm to 800 μm, 100 μm to 700 μm, or 500 μm to 600 μm, e.g., from 1 μm to 10 μm, e.g., 2 μm, 3 μm, 4 μm, 5 μm, 6 μm, 7 μm, 8 μm, 9 μm, or 10 μm, e.g., from 10 μm to 100 μm, e.g., 20 μm, 30 μm, 40 μm, 50 μm, 60 μm, 70 μm, 80 μm, 90 μm, or 100 μm, e.g., from 100 μm to 1 mm, e.g., 200 μm, 300 μm, 400 μm, 500 μm, 600 μm, 700 μm, 800 μm, 900 μm, or 1 mm, e.g., from 1 mm to 10 mm, e.g., 2 mm, 3 mm, 4 mm, 5 mm, 6 mm, 7 mm, 8 mm, 9 mm, or 10 mm, e.g., from 10 mm to 100 mm, e.g., 20 mm, 30 mm, 40 mm, 50 mm, 60 mm, 70 mm, 80 mm, 90 mm, or 100 mm) in diameter. For example, in some embodiments, each microwell (4) is from 10 μm to 100 mm in diameter. In some embodiments, each microwell (4) is from 20 μm to 10 mm in diameter. In some embodiments, each microwell 30 μm to 1 mm in diameter. In some embodiments, each microwell (4) is from 40 μm to 900 μm in diameter. In some embodiments, each microwell (4) is from 50 μm to 800 μm in diameter. In some embodiments, each microwell (4) is from 100 μm to 700 μm in diameter. In some embodiments, each microwell (4) is from 500 μm to 600 μm in diameter.

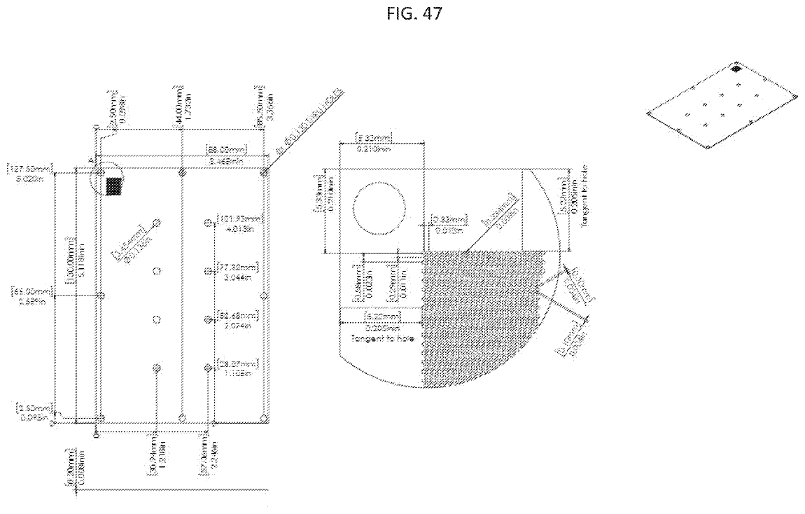

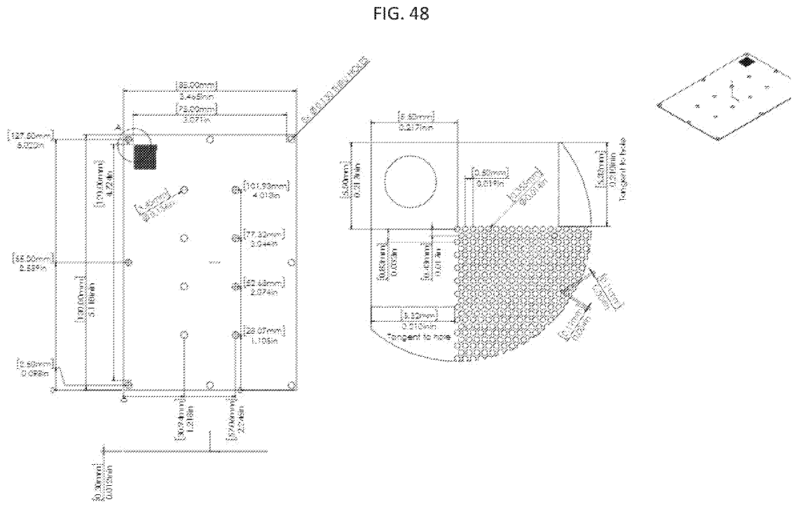

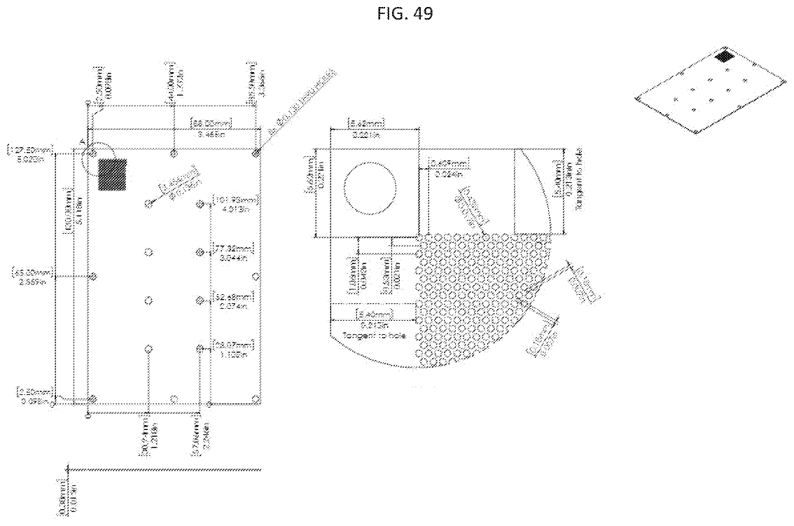

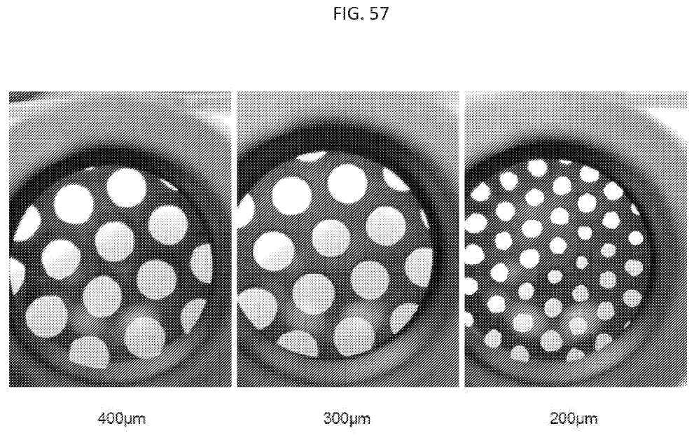

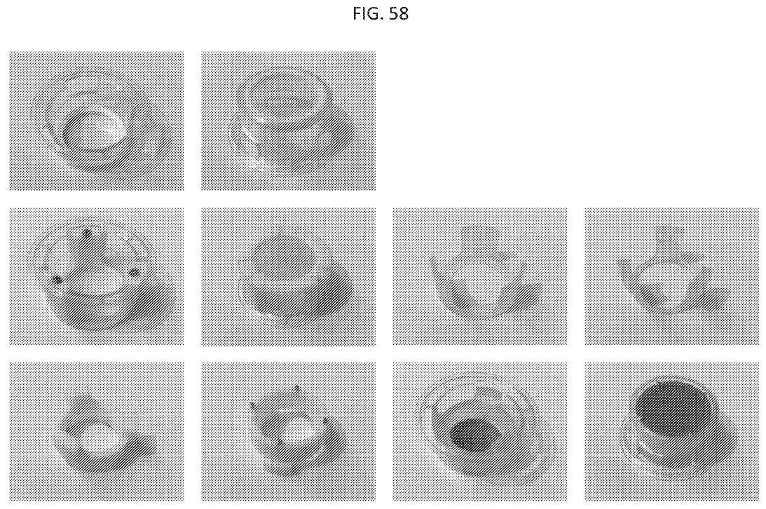

In some embodiments, each microwell is 200 μm in diameter. In some embodiments, each microwell is 234 μm in diameter (FIG. 47). In some embodiments, each microwell is 300 μm in diameter. In some embodiments, each microwell is 355 μm in diameter (FIG. 48). In some embodiments, each microwell is 400 μm in diameter. In some embodiments, each microwell is 432 μm in diameter (FIG. 49). In some embodiments, each microwell is 440 μm in diameter (see, e.g., FIG. 57 for exemplary microwell diameters and FIG. 58 for exemplary microwell components).

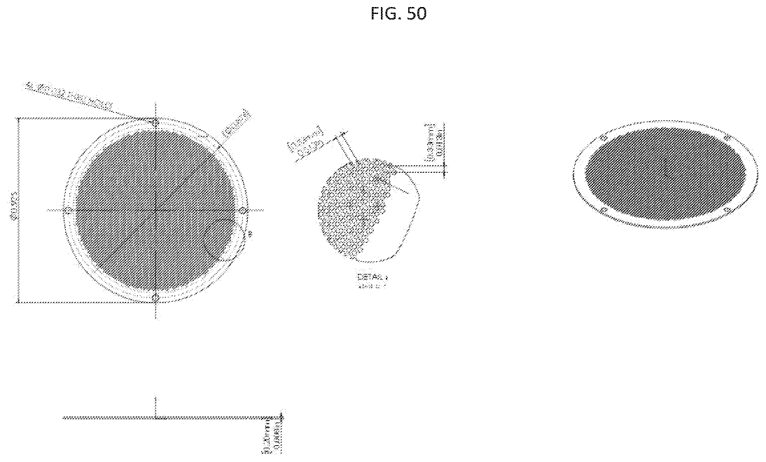

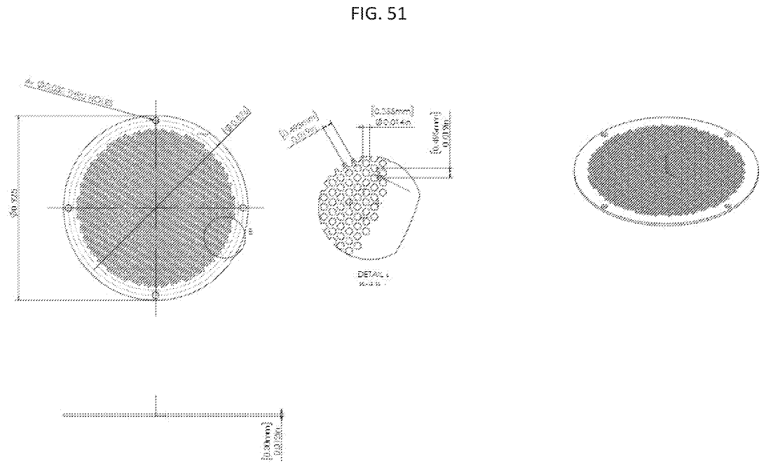

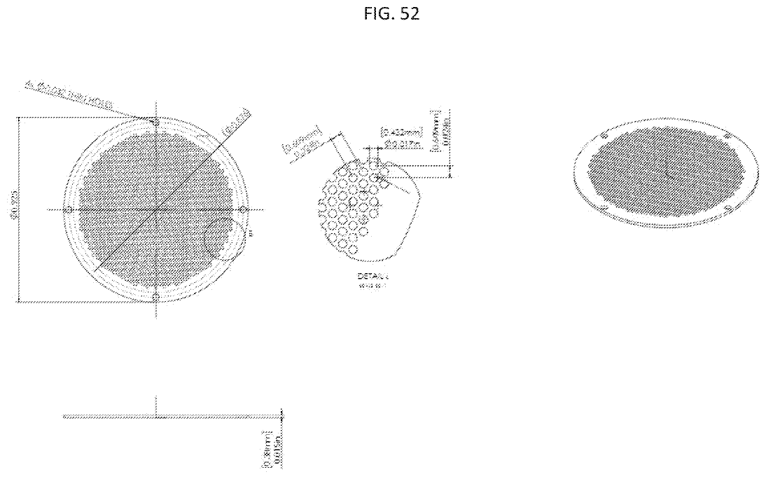

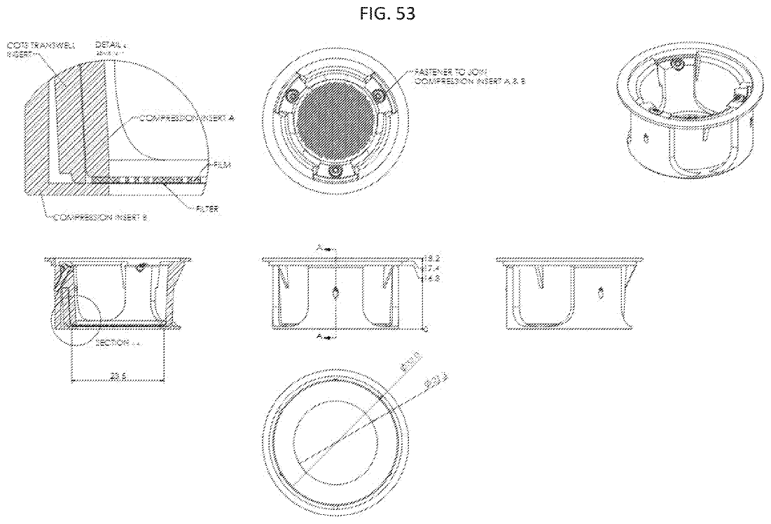

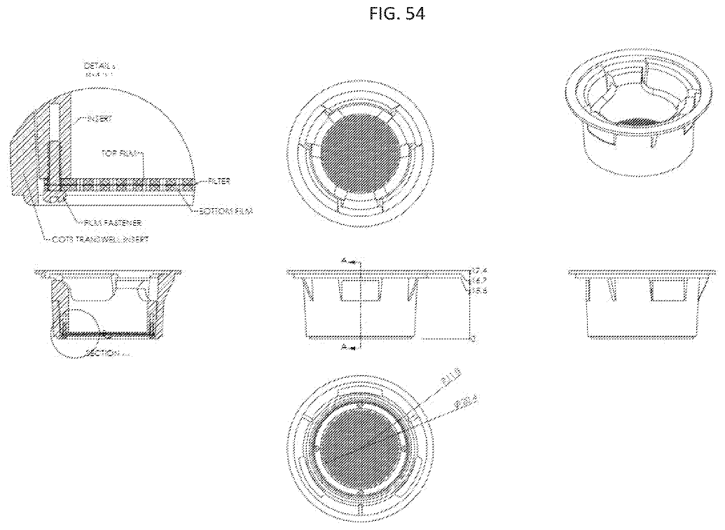

In some embodiments, each microwell is spaced from 100 μm to 2,000 μm apart from each other. For example, in some embodiments, each microwell is spaced 100 μm, 200 μm, 300 μm, 400 μm, 500 μm, 600 μm, 700 μm, 800 μm, 900 μm, 1,000 μm, 1,200 μm, 1,300 μm, 1,400 μm, 1,500 μm, 1,600 μm, 1,700 μm, 1,800 μm, 1,900 μm, or 2,000 μm (see, e.g., FIGS. 50-54). In some embodiments, each microwell is spaced 600 μm apart. In some embodiments, each microwell is spaced 800 μm apart. In some embodiments, each microwell is spaced 880 μm apart. In some embodiments, each microwell is spaced 1,000 μm apart.

In some embodiments, each microwell is 300 μm in diameter and spaced 600 μm apart. In some embodiments, each microwell is 400 μm in diameter and spaced 1,000 μm apart. In some embodiments, each microwell is 400 μm in diameter and spaced 800 μm apart. In some embodiments, each microwell is 440 μm in diameter and spaced 880 μm apart. In some embodiments, each microwell is 400 μm in diameter and spaced 1,000 μm apart. In some embodiments, each microwell is 400 μm in diameter and spaced 1,000 μm apart.

In some embodiments, the microwells (4) include a stainless material (e.g., stainless steel) or a polymer. For example, in some embodiments, the microwells (4) include a stainless material (e.g., stainless steel). In some embodiments, the microwells (4) include a polymer (e.g., polystyrene, PET, polycarbonate, polypropylene, cellulose acetate, PES, liquid silicone rubber (e.g., ELASTOSIL® LR 3003/30 A/B), or fluorinated ethylene propylene (FEP)). In some embodiments, the microwells (4) include polystyrene.

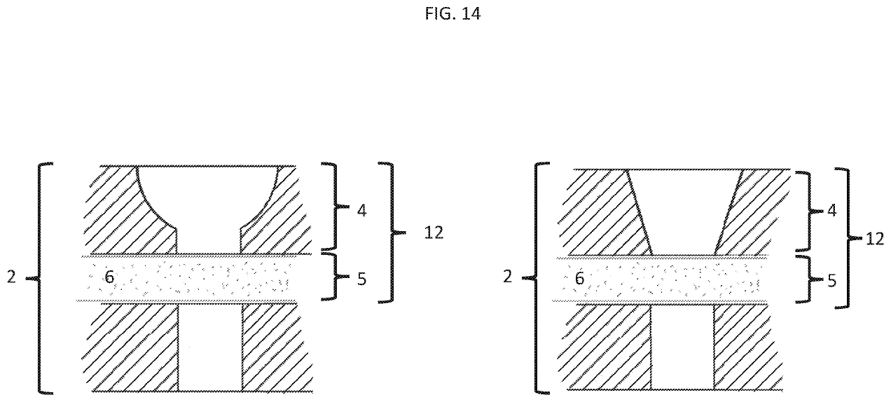

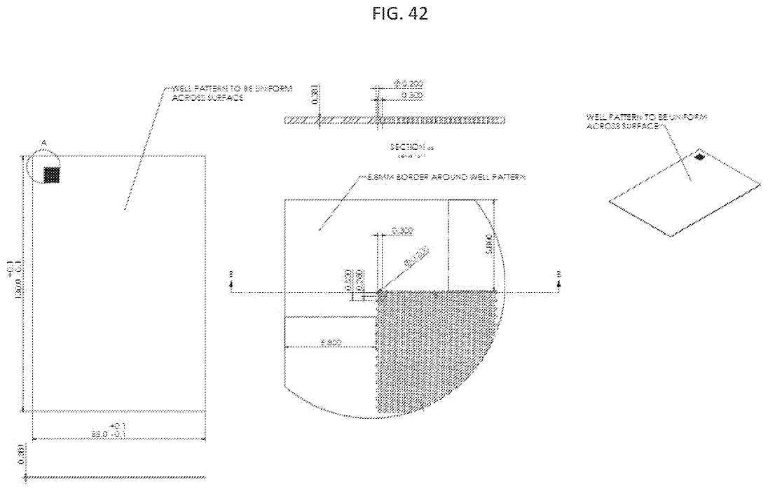

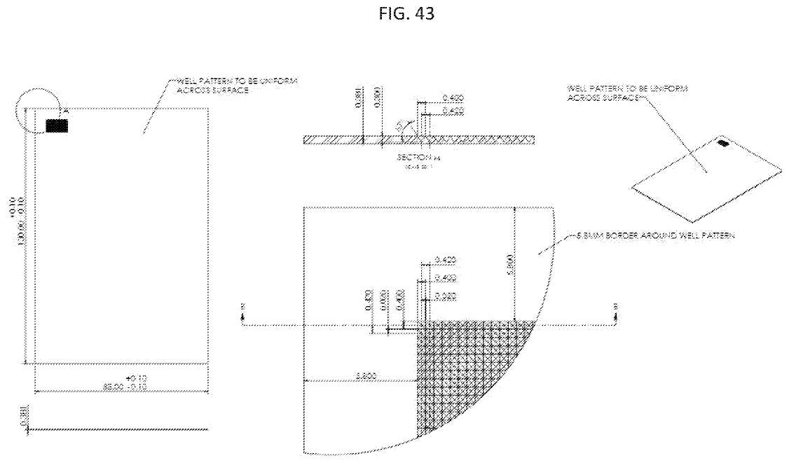

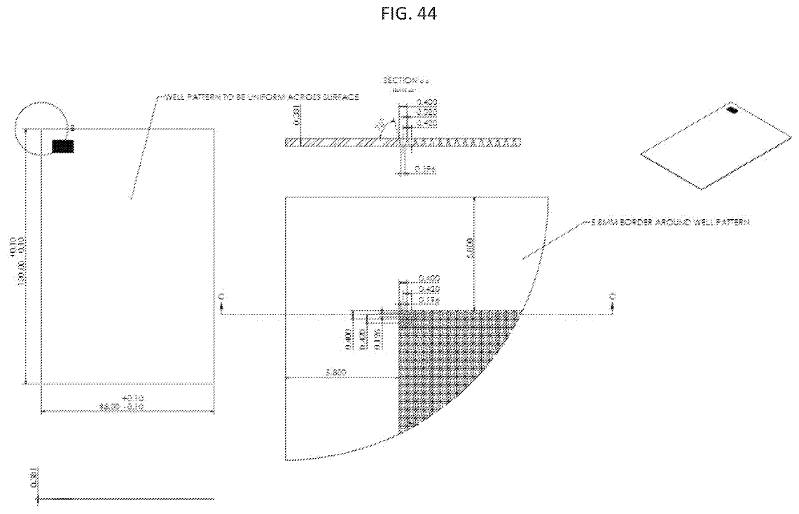

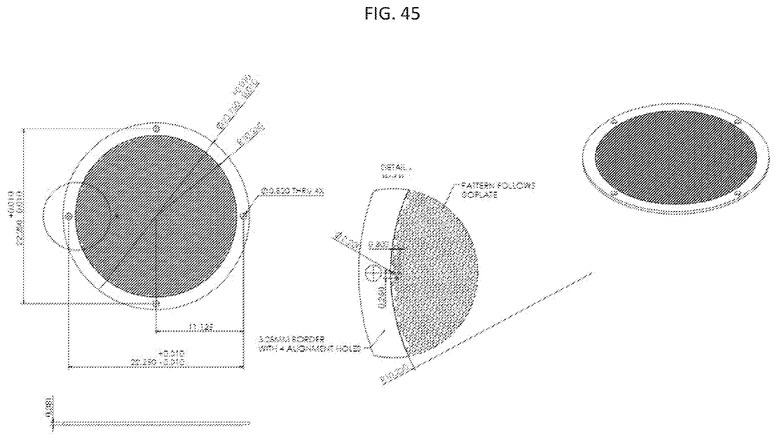

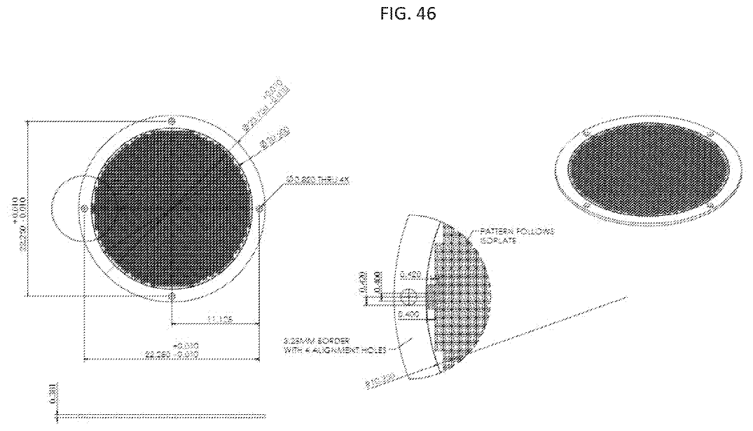

In some embodiments, the microwells (4) are conical, spherical, cylindrical, pyramidal, or chaliced shaped (FIGS. 14 and 42-46). For example, in some embodiments, the microwells (4) are conical. In some embodiments, the microwells (4) are spherical. In some embodiments, the microwells (4) are cylindrical (FIGS. 42 and 45). In some embodiments, the microwells (4) are pyramidal (FIG. 43). In some embodiments, the microwells (4) are square. In some embodiments, the microwells (4) are square with a side taper (FIGS. 44 and 46).

In some embodiments, the passivation coating (8) includes a non-covalent coating (e.g., a block co-polymer, polyethylene glycol (PEG), streptavidin, albumin/biotin, phospholipid surfactants, hyaluronic acid, or poly-lysine-based adherence) and/or a covalent coating (e.g., a covalent attachment e.g., carboxylic acid/amine bonds, disulfide bonds, n-hydroxysuccinimide (NHS) esters, NHS, maleimide, cycloadditions, epoxy, amine, carboxy, aldehyde, p-phenylene diisothiocyanate (PDITC), maleimide, thiol, poly-l-lysine, streptavidin, or neutravidin).

For example, in some embodiments, the passivation coating (8) includes a non-covalent coating. In some embodiments, the passivation coating (8) includes a block co-polymer. In some embodiments, the passivation coating (8) includes PEG. In some embodiments, the passivation coating (8) includes streptavidin. In some embodiments, the passivation coating (8) includes albumin/biotin. In some embodiments, the passivation coating (8) includes phospholipid surfactants. In some embodiments, the passivation coating (8) includes hyaluronic acid. In some embodiments, the passivation coating (8) includes a poly-lysine-based adherence.

In some embodiments, the passivation coating (8) includes a covalent coating. For example, in some embodiments, the passivation coating (8) includes a covalent attachment (e.g., carboxylic acid/amine bonds, disulfide bonds, n-hydroxysuccinimide (NHS) esters, NHS, maleimide, cycloadditions, epoxy, amine, carboxy, aldehyde, p-phenylene diisothiocyanate (PDITC), maleimide, thiol, poly-l-lysine, streptavidin, or neutravidin). In some embodiments, the passivation coating (8) includes carboxylic acid/amine bonds. In some embodiments, the passivation coating (8) includes disulfide bonds. In some embodiments, the passivation coating (8) includes NHS esters. In some embodiments, the passivation coating (8) includes NHS. In some embodiments, the passivation coating (8) includes maleimide. In some embodiments, the passivation coating (8) includes cycloadditions. In some embodiments, the passivation coating (8) includes epoxy. In some embodiments, the passivation coating (8) includes amine. In some embodiments, the passivation coating (8) includes carboxy. In some embodiments, the passivation coating (8) includes aldehyde. In some embodiments, the passivation coating (8) includes PDITC. In some embodiments, the passivation coating (8) includes maleimide. In some embodiments, the passivation coating (8) includes thiol. In some embodiments, the passivation coating (8) includes poly-l-lysine. In some embodiments, the passivation coating (8) includes streptavidin. In some embodiments, the passivation coating (8) includes neutravidin.

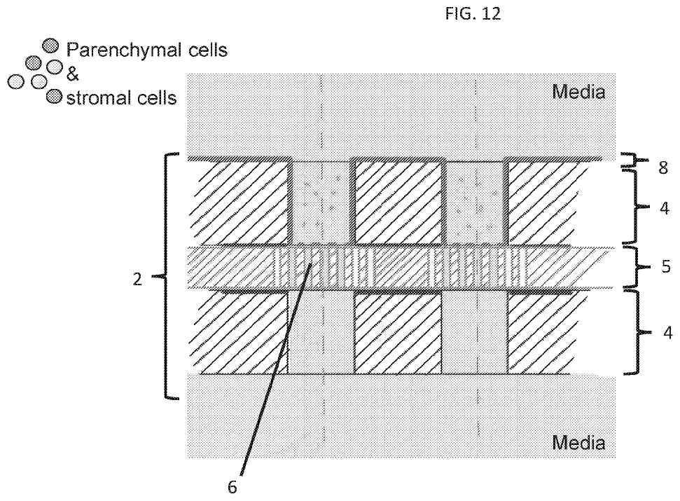

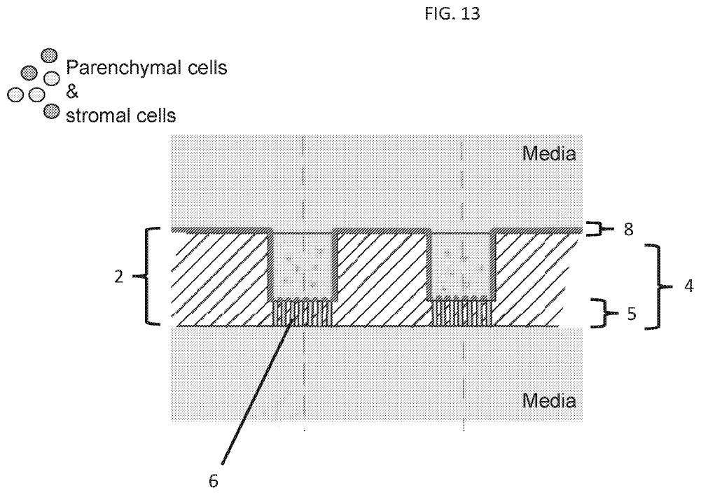

In some embodiments, the microwell film composite includes a second layer that includes a Permeable body (5) that may include pores (6) (FIGS. 12 and 13). In some embodiments, the permeable body is a porous filter. In some embodiments, the pores are formed by non-woven non-linear pathways. In some embodiments, the first layer including a plurality of microwells (4) and a second layer including a permeable body (5) are components of a laminate (12).

In some instances, the permeable body (5) may include a polymer (e.g., polycarbonate, polyester, polystyrene, polytetrafluoroethylene (PTFE), collagen-coated PTFE, polyethylene terephthalate (PET), polysulfone (PES), nylon, or cellulose acetate), or liquid silicone rubber (e.g., ELASTOSIL® LR 3003/30 A/B). In some embodiments, the permeable body may include stainless steel. For example, in some embodiments, the permeable body (5) includes polycarbonate. In some embodiments, the permeable body (5) includes polyester. In some embodiments, the permeable body (5) includes polystyrene. In some embodiments, the permeable body (5) includes PTFE. In some embodiments, the permeable body (5) includes collagen-coated PTFE.

In some embodiments, each pore (6) may have a size of from 0.1 μm to 15 μm (e.g., 0.2 μm to 14 μm, 0.3 μm to 13 μm, 0.4 μm to 12 μm, 0.5 μm to 11 μm, 1 μm to 10 μm, or 5 μm). Such a size will enable perfusion of a fluid including media through the pathway but prevent the passage of cells through the pathway. In particular, each pore (6) is configured to allow flow of the perfusion medium and prevent flow of stromal cells or parenchymal cells (FIGS. 12 and 13).

For example, in some embodiments, each pore (6) may have a size of from 0.2 μm to 14 μm. In some embodiments, each pore (6) may have a size of from 0.3 μm to 13 μm. In some embodiments, each pore (6) may have a size of from 0.4 μm to 12 μm. In some embodiments, each pore (6) may have a size of from 0.5 μm to 11 μm. In some embodiments, each pore (6) may have a size of from 1 μm to 10 μm. In some embodiments, each pore (6) may have a size of 5 μm.

In some embodiments, each pore (6) may have a size of from 0.1 μm to 3 μm (e.g., 0.2 μm to 2 μm, 0.3 μm to 1 μm, 0.4 μm to 0.9 μm, 0.5 μm to 0.8 μm, or 0.6 μm to 0.7 μm) in diameter. In some embodiments, each pore (6) may have a size of from 0.2 μm to 2 μm in diameter. In some embodiments, each pore (6) may have a size of from 0.3 μm to 1 μm in diameter. In some embodiments, each pore (6) may have a size of from 0.4 μm to 0.9 μm in diameter. In some embodiments, each pore (6) may have a size of from 0.5 μm to 0.8 μm in diameter. In some embodiments, each pore (6) may have a size of from 0.6 μm to 0.7 μm in diameter.

In some embodiments, the permeable body (5) has a density of pores across the permeable body of from 1×10⁴ pores/cm² to 1×10⁹ pores/cm². For example, the permeably body may have a density of pores across the permeably body of from 10⁴ pores/cm² to 10⁵ pores/cm², e.g., 20,000 pores/cm², 30,000 pores/cm², 40,000 pores/cm², 50,000 pores/cm², 60,000 pores/cm², 70,000 pores/cm², 80,000 pores/cm², 90,000 pores/cm², or 10⁵ pores/cm², e.g., from 10⁵ pores/cm² to 10⁶ pores/cm², e.g., 2×10⁵ pores/cm², 3×10⁵ pores/cm², 4×10⁵ pores/cm², 5×10⁵ pores/cm², 6×10⁵ pores/cm², 7×10⁵ pores/cm², 8×10⁵ pores/cm², 9×10⁵ pores/cm², or 10⁶ pores/cm², e.g., from 10⁶ pores/cm² to 10⁷ pores/cm², e.g., 2×10⁷ pores/cm², 3×10⁷ pores/cm², 4×10⁷ pores/cm², 5×10⁷ pores/cm², 6×10⁷ pores/cm², 7×10⁷ pores/cm², 8×10⁷ pores/cm², 9×10⁷ pores/cm², or 10⁸ pores/cm², e.g., from 10⁸ pores/cm² to 10⁹ pores/cm², e.g., 2×10⁸ pores/cm², 3×10⁸ pores/cm², 4×10⁸ pores/cm², 5×10⁸ pores/cm², 6×10⁸ pores/cm², 7×10⁸ pores/cm², 8×10⁸ pores/cm², 9×10⁸ pores/cm², or 10⁹ pores/cm².

In some embodiments, the laminate further includes a third layer having a perforation film (13) (e.g., polystyrene) (see, e.g., FIG. 16). In some embodiments, the third layer is adjoined to the layer that includes the permeable body (5) and is configured to direct fluid pressure to the microwell film composite (2). For example, in some embodiments, the third layer is polystyrene.

In some embodiments, a first layer that includes a plurality of microwells (4), a second layer including a permeable body (5), and a third layer including a perforation film (13) are components of a laminate (12) in which the microwells (4) includes polystyrene, the permeable body (5) includes PTFE or collagen-coated PTFE, and the perforation film (13) includes polystyrene (FIG. 16). For example, in some embodiments, a first layer that includes a plurality of microwells (4), a second layer including a permeable body (5), and a third layer that includes a perforation film (13) are components of a laminate (12) in which the microwells (4) includes polystyrene, the permeable body (5) includes PTFE, and the perforation film (13) includes polystyrene. In some embodiments, a first layer that includes a plurality of microwells (4), a second layer including a permeable body (5), and a third layer that includes a perforation film (13) are components of a laminate (12) in which the microwells (4) include polystyrene, the permeable body (5) includes collagen-coated PTFE, and the perforation film (13) includes polystyrene.

The layers of the microwell film composite (2) may be joined using any suitable approach. For example, in some embodiments, the layers are joined using an adhesive. The adhesive may have any suitable thickness (e.g., from 1 μm to 100 μm, e.g., 25 μm, 50 μm, or 81 μm). In other embodiments, the layers may be joined using ultrasonic welding.

### Macroscopic Manifold (3)

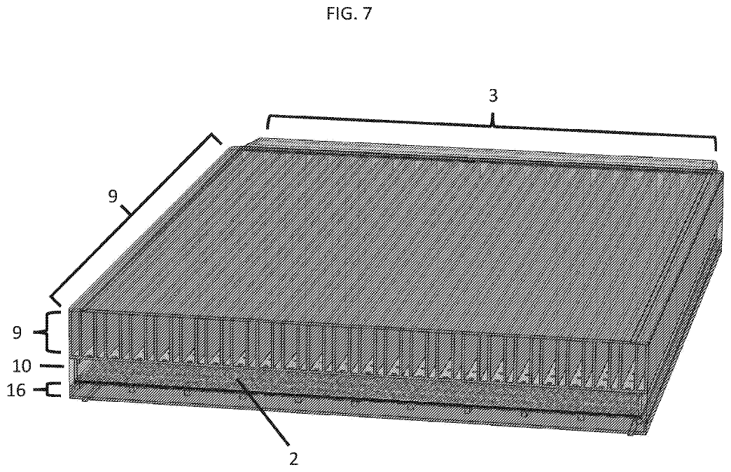

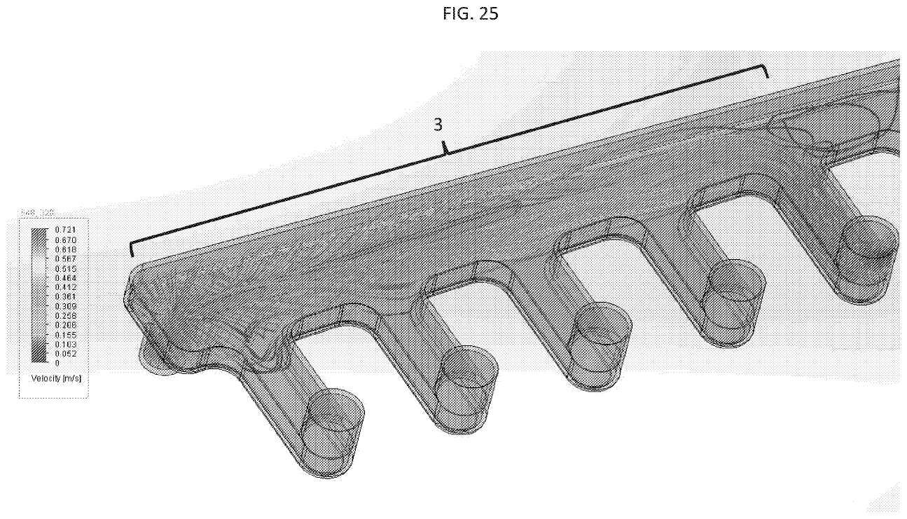

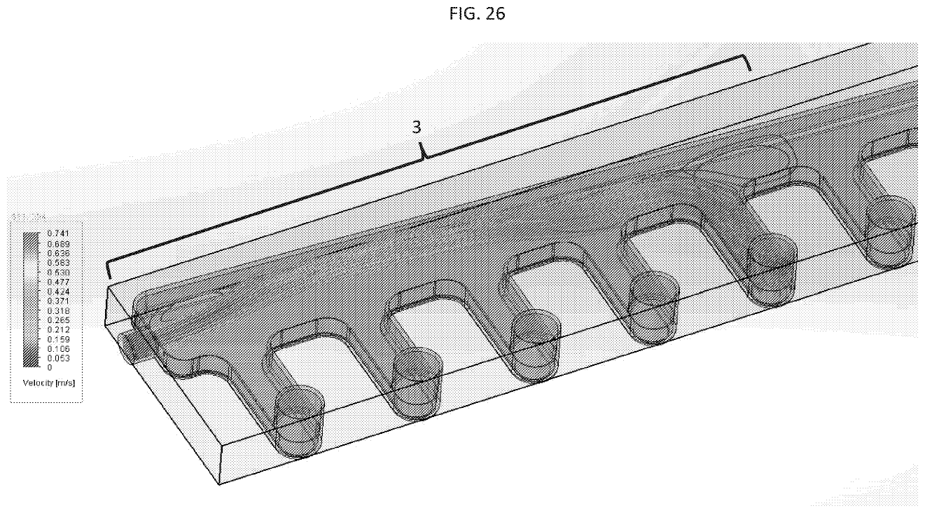

A device described herein may include a macroscopic manifold (3) attached to the plate and in fluid communication with the microwell film composite (2) (FIGS. 4-8). The macroscopic manifold (3) may be connected to the microwell film composite (2) and be configured to direct fluid flow to the microwell film composite (2) (FIGS. 25 and 26).

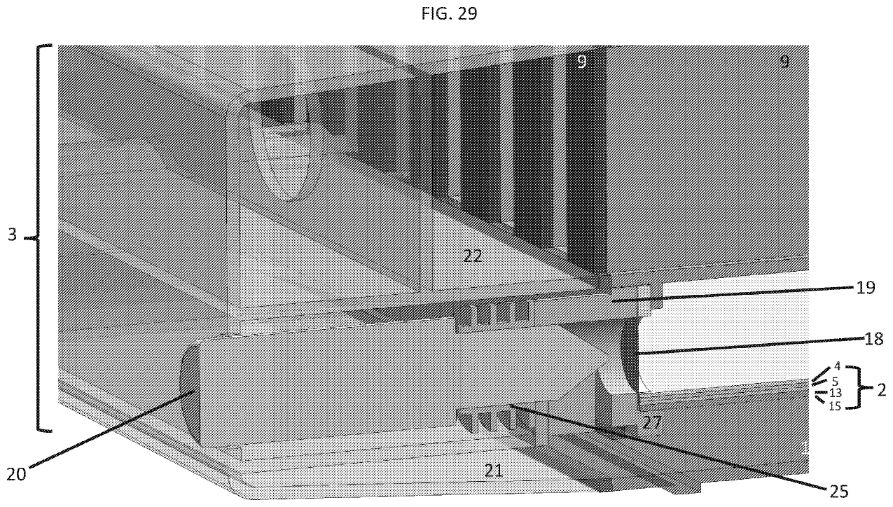

In some embodiments, the macroscopic manifold (3) includes one or more (e.g., 2, 3, 4, 5, or more) foil seals (18) (see, e.g., FIG. 29). Such foil seals (18) may be connected to the film superstructure (27) to direct fluid flow and/or pressure to or from the microwell film composite (2).

In some embodiments, the macroscopic manifold (3) includes one or more (e.g., 2, 3, 4, 5, or more) seal ports (19) (see, e.g., FIG. 29). Such seal ports (19) in fluid connection with the microwell film composite (2), such that the one or more (e.g., 2, 3, 4, 5, or more) seal ports (19) are configured to direct fluid flow and/or pressure to or from the microwell film composite (2).

In some embodiments, the macroscopic manifold (3) includes one or more (e.g., 2, 3, 4, 5, or more) seal punches (20) (see, e.g., FIG. 29). Such seal punches (20) are positioned in the one or more (e.g., 2, 3, 4, 5, or more) seal ports (19), such that the one or more (e.g., 2, 3, 4, 5, or more) seal punches (20) are configured to break the one or more (e.g., 2, 3, 4, 5, or more) foil seals (18).

For example, the one or more foil seals may block fluid flow until they are broken by the one or more seal punches, for example, which will create a passage for fluid through the one or more seal ports (19). For example, aggregates may be collected from the microwell film composite by breaking the one or more foil seals, creating a fluid passage into the seal ports into which the aggregates may flow for collection.

In some embodiments, the macroscopic manifold (3) includes two or more (e.g., 3, 4, 5, or more) seal ports (19) and two or more seal punches (20). For example, in some embodiments, the macroscopic manifold (3) includes three or more seal ports (19) and three or more seal punches (20). In some embodiments, the macroscopic manifold (3) includes four or more seal ports (19) and four or more seal punches (20). In some embodiments, the macroscopic manifold (3) includes five or more seal ports (19) and five or more seal punches (20).

In some embodiments, the macroscopic manifold (3) includes one or more (e.g., 2, 3, 4, 5, or more) seal bellows (25). Such seal bellows (25) connect the one or more (e.g., 2, 3, 4, 5, or more) seal punches (20) to the one or more seal ports (19).

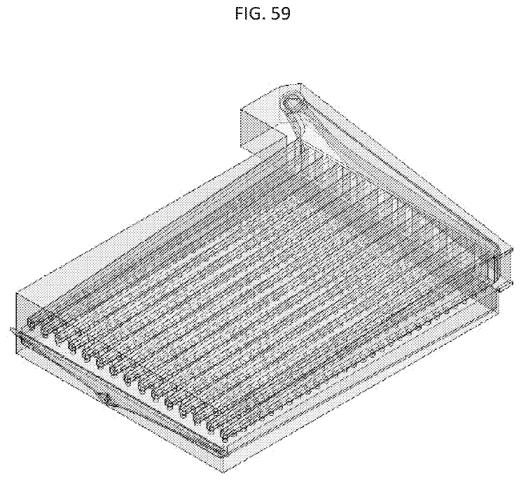

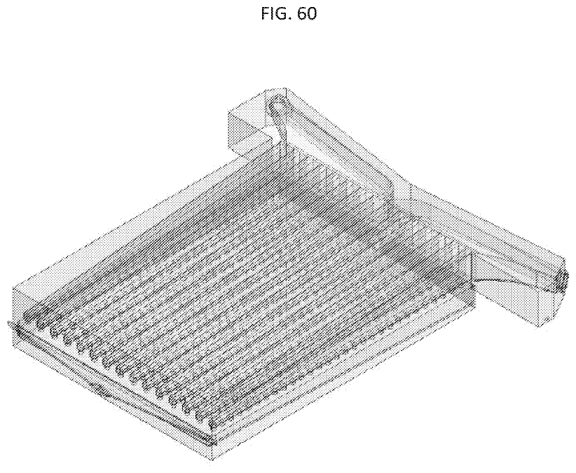

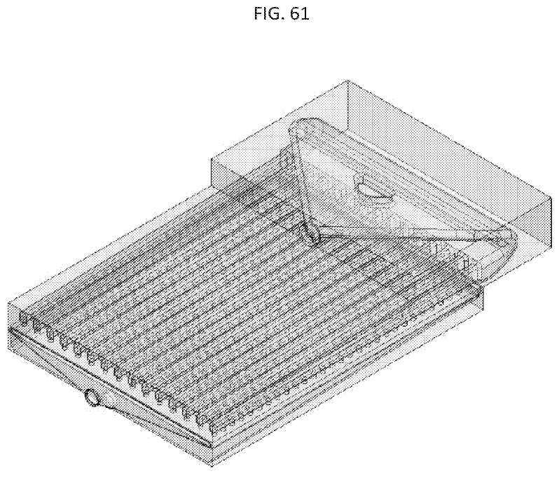

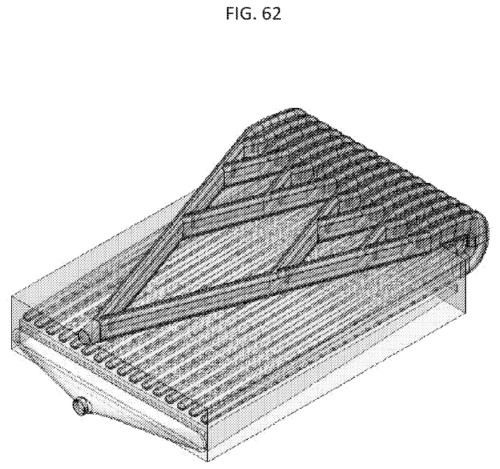

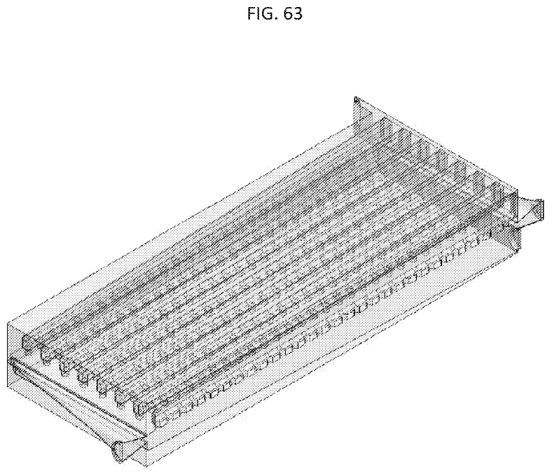

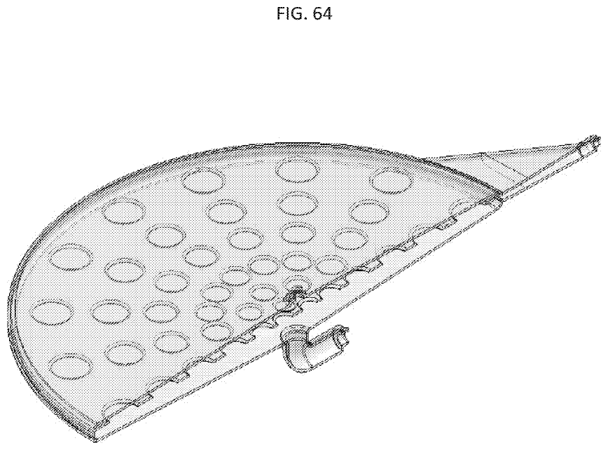

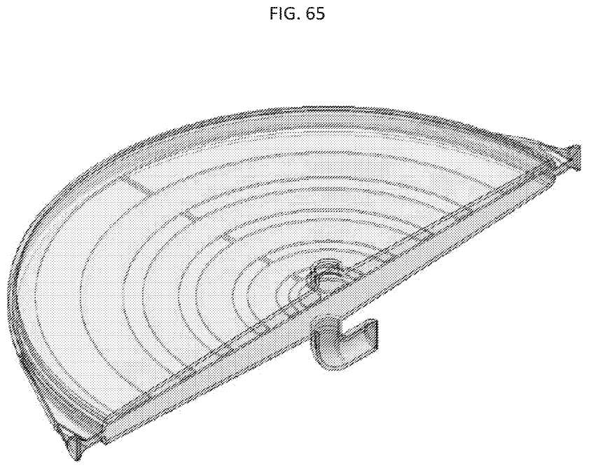

In some embodiments, the macroscopic manifold (3) includes a sealed outlet (21). Such a sealed outlet (21) is in fluid connection with the one or more seal ports (19), such that the sealed outlet (21) is configured to direct fluid flow from the microwells (4) to the one or more seal ports (19). In some embodiments, the macroscopic manifold (3) includes a sealed inlet (22). Such a sealed inlet (22) is in fluid connection with the one or more seal ports (19), such that the sealed inlet (22) is configured to direct fluid flow from the microwells (4) to the one or more seal ports (19). The macroscopic manifold may include any suitable geometry to connect to the microwell film composite (2) and be configured to direct fluid flow to the microwell film composite (2) (see, e.g., FIGS. 59-65). For example, the macroscopic manifold may be an angled inlet manifold (FIG. 59), a dual inlet manifold (FIG. 60), a loop inlet manifold (FIG. 61), a bifurcation manifold (FIG. 62), a radial manifold (FIG. 64), or a radial channel manifold (FIG. 65).

### Additional Elements

A device described herein may additionally include elements such as a gasket (17), a cam bar (23), and/or an actuator (e.g., a button) (24), which are described in the sections that follow.

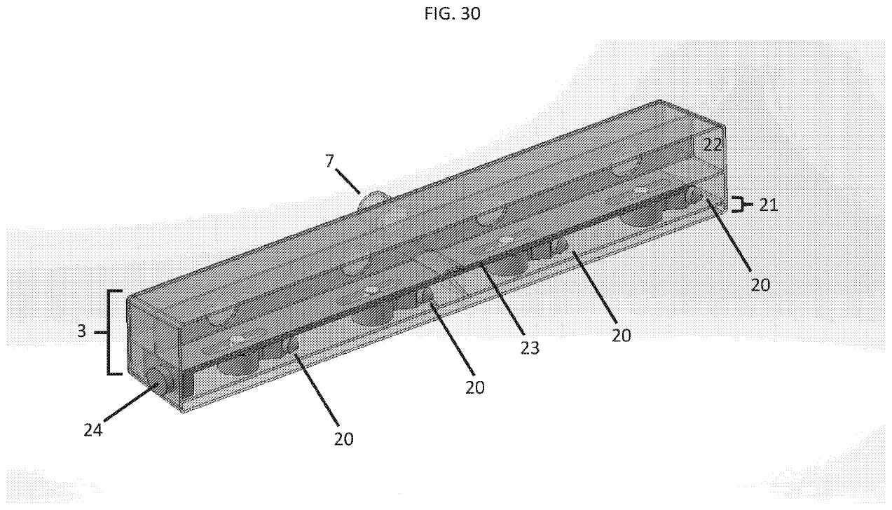

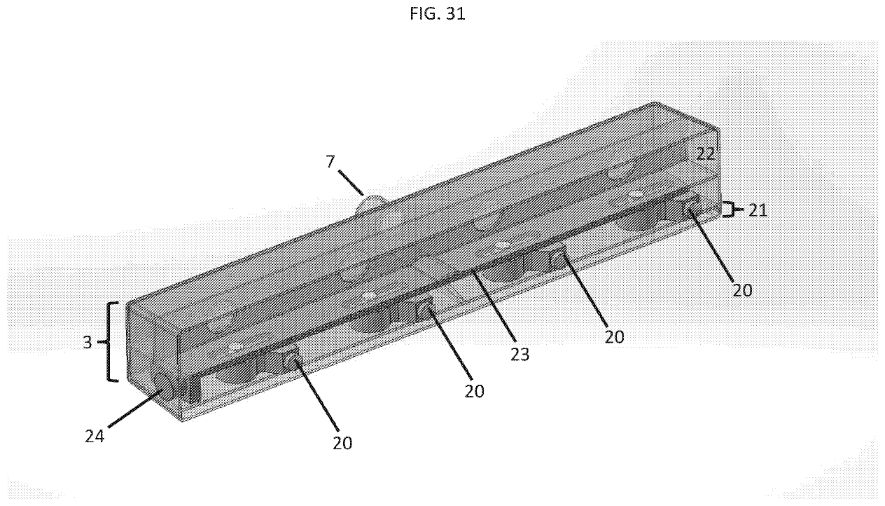

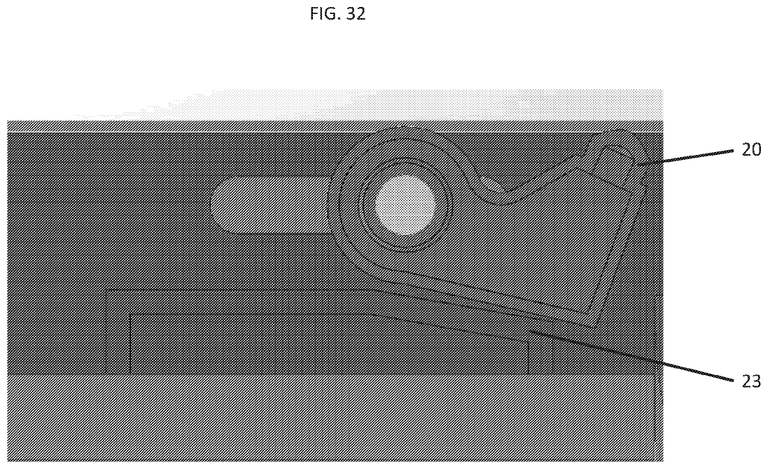

In some embodiments, a device described herein may include a cam bar (23) (see, e.g., FIGS. 30-32), which may be connected to the one or more seal punches (20). The cam bar may, for example, allow for engagement of a plurality of seal punches by an actuator (24).

In some embodiments, a device described herein may include an actuator (24), which may be connected to the cam bar (24) and configured to engage the one or more seal punches (20). In some embodiments, the cam bar is connected to the macroscopic manifold (3). The actuator may be operably connected to the cam bar, which actuates the seal punches to break the foil seals in the seal ports. The actuator may be activated, e.g., by pushing the actuator (e.g., a button or a lever).

In some embodiments, a device described herein may include one or more (e.g., 2, 3, 4, 5, or more) gaskets (17). The one or more gaskets (17) may form a seal between the macroscopic manifold (3) and the microwell film composite (2).

### Methods of Aggregation

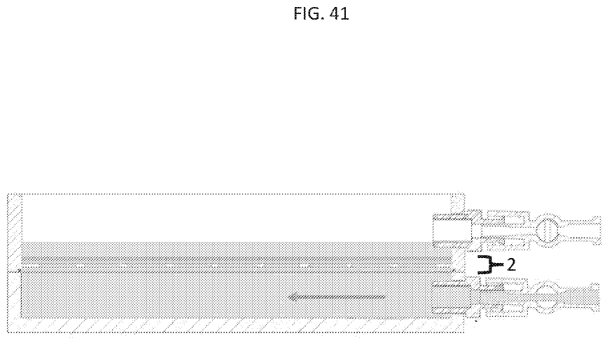

The invention features a method of using a device as described herein. The methods employ using a device for aggregating a population of cells, such as a population of stromal cells and/or a population of parenchymal cells. The methods of aggregation may include aggregating a population of stromal cells and a population of parenchymal cells. The method includes the steps of (a) providing a device as described herein and a medium (e.g., perfusion medium) that includes a population of cells (e.g., a first population of cells and a second population of cells, e.g., a first population of stromal cells and a second population of parenchymal cells); and (b) introducing the medium (e.g., perfusion medium) that includes the population of cells (e.g., the first population of cells and the second population of cells, e.g., the first population of stromal cells and the second population of parenchymal cells) into the device through one of the ports (7) to direct the medium (e.g., perfusion medium) that includes the cells into the microwells in the microwell film composite (2) (FIGS. 17-21 and 36-41). The aggregates may include, for example, two or more populations of cells (e.g., two, three, four, five, six, seven, eight, nine, ten, or more populations of cells).

In some embodiments the first population of cells and/or the second population of cells are induced pluripotent (iPSC)-derived cells, engineered cells, primary cells, embryonic stem cells (ESC)-derived cells, or transdifferentiated cells.

The introduction of the first population of cells and the second population of cells can be sequential, e.g., the first population of cells may be introduced, e.g., in a first medium, and the second population of cells may be introduced, e.g., in a second medium. Similarly, the second population of cells may be introduced, e.g., in a second medium, and the first population of cells may be introduced, e.g., in a first medium. Alternatively, the first population of cells and the second population of cells may be introduced in the same medium.

The introduction of stromal cells and parenchymal cells can be sequential, e.g., the stromal cells may be introduced, e.g., in a first medium, and the parenchymal cells may be introduced, e.g., in a second medium. Similarly, the parenchymal cells may be introduced, e.g., in a first medium, and the stromal cells may be introduced, e.g., in a second medium. Alternatively, the stromal cells and the parenchymal cells may be introduced in the same medium.

The media used to culture cells may be any suitable cell culture media. Exemplary media components used to culture the cells is shown in Table 1 below.

TABLE 1
Cell culture media and supplements

| Base Media | Supplements |
|---|---|
| 4.5 g/L Dulbecco's | 10% heat inactivated fetal bovine serum |
| Modified Eagle | 6.4 μg/mL insulin |
| Medium (DMEM) +L- | 6.4 μg/mL transferrin |
| glutamine, −sodium | 6.4 ng/mL selenous acid |
| pyruvate | 1.3 mg/mL bovine serum albumin |
| | 5.5 μg/mL linoleic acid |
| | 15 mM HEPES |
| | 40 ng/mL Glucagon |
| | 60 ng/mL Dexamethasone |
| | 100 U/mL Penicillin |
| | 100 μg/mL Streptomycin |
| 4.5 g/L DMEM +L- | 10% heat inactivated fetal bovine serum |
| glutamine, −sodium | 6.4 μg/mL insulin |
| pyruvate | 6.4 μg/mL transferrin |
| | 6.4 ng/mL selenous acid |
| | 1.3 mg/mL bovine serum albumin |
| | 5.5 μg/mL linoleic acid |
| | 15 mM HEPES |
| | 40 ng/mL Glucagon |
| | 60 ng/mL Dexamethasone |
| | 100 U/mL Penicillin |
| | 100 μg/mL Streptomycin |
| | 10 μM Y-27632 ROCK inhibitor |
| 4.5 g/L DMEM +L- | 10% heat inactivated fetal bovine serum |
| glutamine, −sodium | 6.4 μg/mL insulin |
| pyruvate | 6.4 μg/mL transferrin |
| | 6.4 ng/mL selenous acid |
| | 1.3 mg/mL bovine serum albumin |
| | 5.5 μg/mL linoleic acid |
| | 15 mM HEPES |
| | 40 ng/mL Glucagon |
| | 60 ng/mL Dexamethasone |
| | 100 U/mL Penicillin |
| | 100 μg/mL Streptomycin |
| | 5 μg/mL laminin |
| 4.5 g/L DMEM +L- | 1% human platelet lysate |
| glutamine, −sodium | 40 ng/mL Glucagon |
| pyruvate | 60 ng/mL Dexamethasone |
| | 10 μM Y-27632 ROCK inhibitor |
| 4.5 g/L DMEM +L- | 5% human platelet lysate |
| glutamine, −sodium | 40 ng/mL Glucagon |
| pyruvate | 60 ng/mL Dexamethasone |
| | 10 μM Y-27632 ROCK inhibitor |
| 4.5 g/L DMEM +L- | 1% human serum |
| glutamine, −sodium | 40 ng/mL Glucagon |
| pyruvate | 60 ng/mL Dexamethasone |
| | 10 μM Y-27632 ROCK inhibitor |
| 4.5 g/L DMEM +L- | 5% human serum |
| glutamine, −sodium | 40 ng/mL Glucagon |
| pyruvate | 60 ng/mL Dexamethasone |
| | 10 μM Y-27632 ROCK inhibitor |
| 4.5 g/L DMEM +L- | 10% human serum |
| glutamine, −sodium | 40 ng/mL Glucagon |
| pyruvate | 60 ng/mL Dexamethasone |
| | 10 μM Y-27632 ROCK inhibitor |

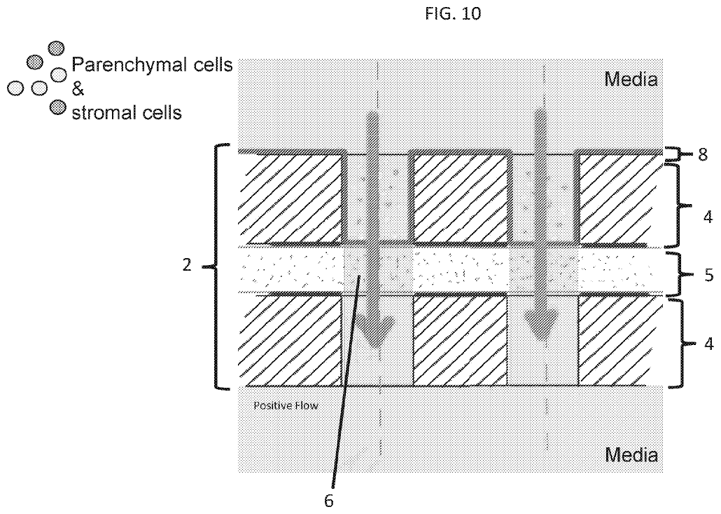

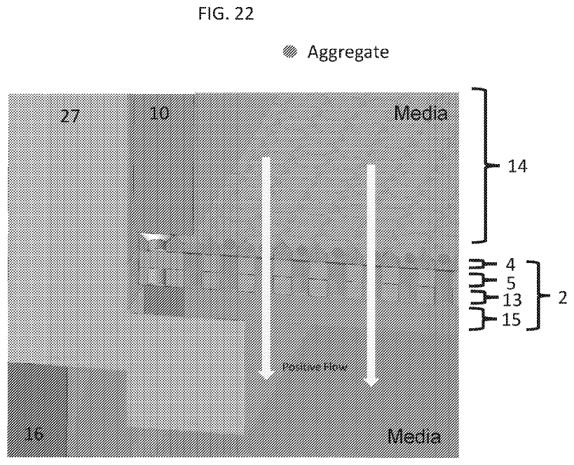

In some embodiments, the device includes a macroscopic manifold (3) connected to the microwell film composite (2), and the method includes applying a positive pressure to the macroscopic manifold to direct fluid flow to the microwell film composite (FIGS. 10 and 22).

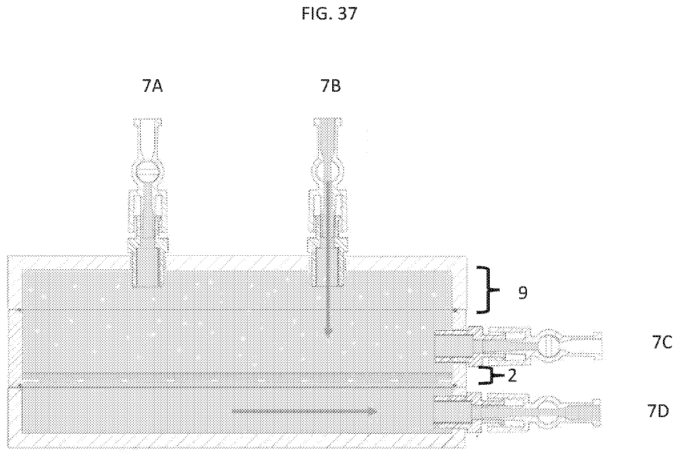

Other methods may be used to apply the cells into the microwells. For example, in some embodiments, the method includes shaking or centrifuging the device, e.g., to settle the cells in the microwells. The pores (6) allow flow of the aggregation medium while preventing flow of cells (e.g., stromal cells or parenchymal cells) through the permeable body (5) (FIG. 37).

In some embodiments, the method includes introducing an aggregation medium through a port (7) (FIG. 36).

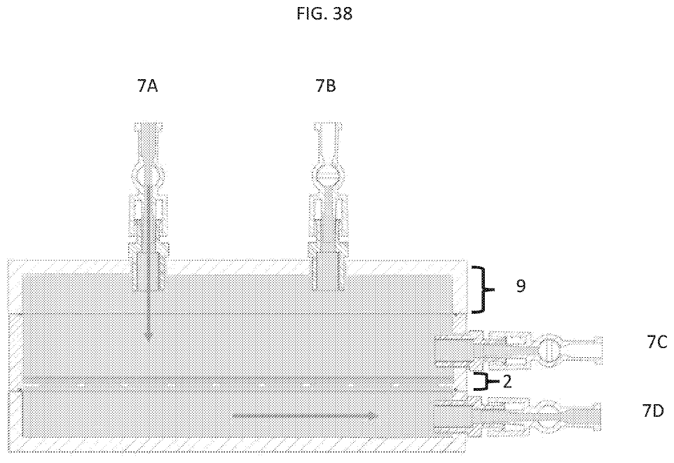

In some embodiments, the method includes applying a pressure to the macroscopic manifold to direct the aggregation medium into the microwells of the microwell film composite (2) (FIG. 38). In some embodiments, the method further includes culturing the cells (e.g., stromal cells and parenchymal cells) in the microwells under conditions (e.g., perfusion conditions) such that aggregates of stromal cells and parenchymal cells form in the microwells.

In some embodiments, the method includes culturing the stromal cells and the parenchymal cells in the microwells under conditions (e.g., perfusion conditions) in the aggregation medium such that aggregates of stromal cells and parenchymal cells form in the microwells.

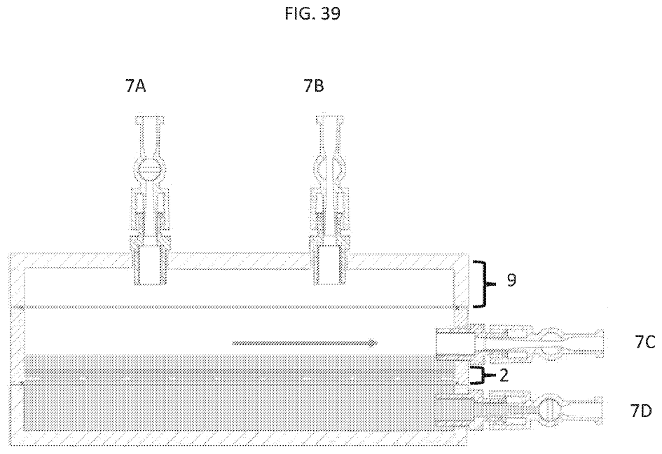

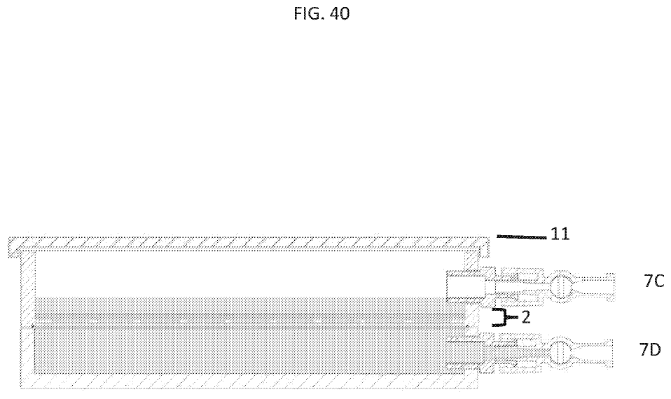

In some embodiments, the method further includes washing or removing the aggregation medium. Washing or removing a medium may include pipetting, decanting, draining, or replacing the medium, e.g., via flow through a port (7) (FIGS. 39 and 40).

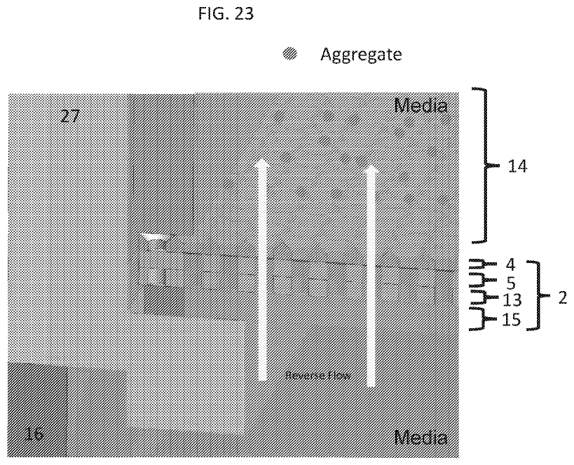

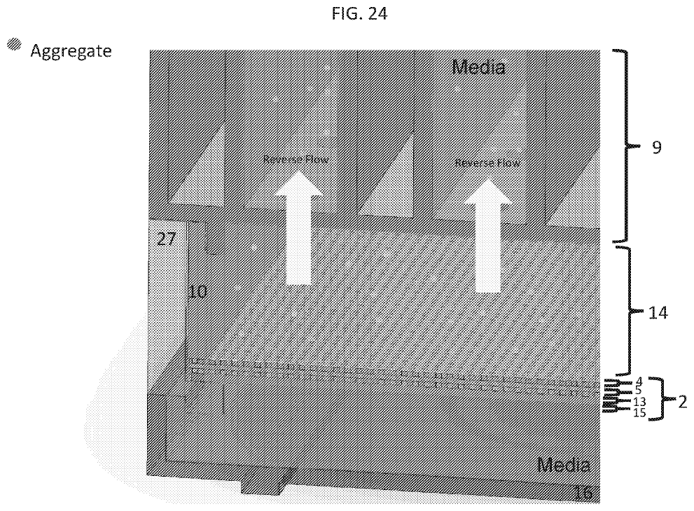

In some embodiments, the method further includes releasing the aggregates from the microwells, thereby producing a population of aggregates including stromal cells and parenchymal cells (FIGS. 23, 24 and 41). Such releasing may include, for example, applying a negative pressure to the macroscopic manifold to release the aggregates (FIG. 11). In other embodiments, the method includes applying a negative pressure to the upper manifold (FIG. 24). Alternatively, this step may include decanting or pipetting the aggregates to release the aggregates. The method may further include collecting the population of aggregates (e.g., by applying a negative pressure).

In some embodiments, the device includes a collection outlet in fluid communication with the first layer that includes the plurality of microwells, and the method includes collecting the population of aggregates as it passes through the collection outlet. The collection outlet may be, e.g., a port (7), such as port 7C in FIG. 37.

In some embodiments, the one or more plates include an upper manifold (9). The upper manifold is in fluid connection with the macroscopic manifold and directs fluid flow and/or fluid distribution to or from the macroscopic manifold (3) to the microwell film composite (2).

In some embodiments, the macroscopic manifold (3) further includes one or more seal ports (19) in fluid connection with the microwell film composite (2). The one or more seal ports (19) direct fluid flow and/or pressure to or from the microwell film composite (2).

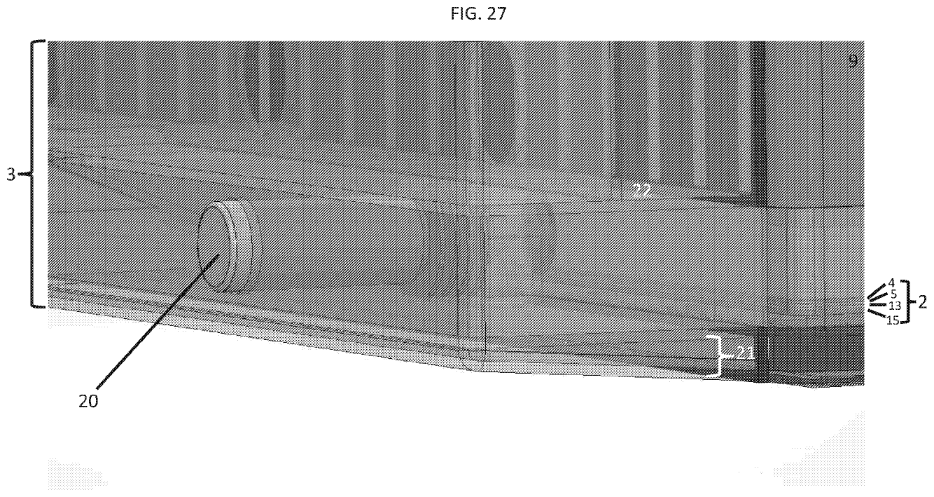

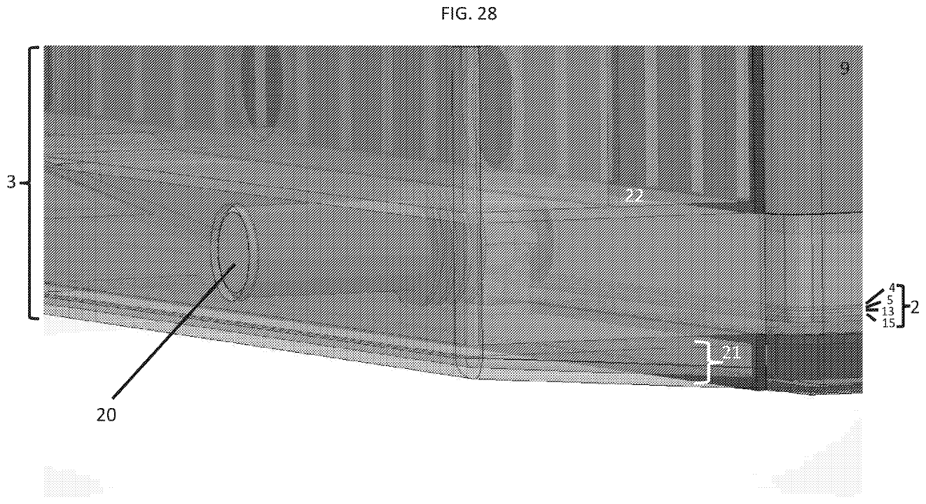

In some embodiments, the macroscopic manifold (2) further includes one or more foil seals (18) attached to the seal ports (19) (FIG. 29). The macroscopic manifold (3) may further include one or more seal punches (20) positioned in the one or more seal ports, and the seal punches break the one or more foil seals (FIGS. 27 and 28). In some embodiments, the device further includes a cam bar (23) connected to the one or more seal punches (20), and the method includes actuating the cam bar to engage the one or more seal punches to break the one or more foil seals (FIGS. 30-32). In such embodiments, the aggregates may be collected by breaking the one or more foil seals (18) with the one or more seal punches (20) and collecting the aggregates via the seal ports (19) (see, e.g., FIG. 29).

### Cell Populations

Cell populations may be optimized to maintain the appropriate morphology, phenotype, and cellular function conducive to use in the methods and devices of the disclosure. For example, primary human hepatocytes or neonatal foreskin stromal cells can be isolated and/or pre-cultured under conditions optimized to ensure that the respective cells of choice initially have the desired morphology, phenotype, and cellular function and, thus, are poised to maintain said morphology, phenotype and/or function while being aggregated in a device described herein.

In some embodiments, a method described herein may include providing a first population of cells and a second population of cells.

In some embodiments, a method described herein may include providing a population of parenchymal cells (e.g., hepatocytes, pancreatic exocrine cells, myocytes, pancreatic endocrine cells, neurons, enterocytes, adipocytes, splenic cells, kidney cells, biliary cells, Kupffer cells, stellate cells, cardiac muscle cells, alveolar cells, bronchiolar cells, club cells, urothelial cells, mucous cells, parietal cells, chief cells, G cells, goblet cells, enteroendocrine cells, Paneth cells, M cells, tuft cells, glial cells, gall bladder cells, keratinocytes, melanocytes, Merkel cells, Langerhans cells, osteocytes, osteoclasts, esophageal cells, photoreceptor cells, and corneal epithelial cells). In some embodiments, the parenchymal cells are pancreatic cells (e.g., alpha, beta, gamma, delta, epsilon cells, or any combination thereof). In some embodiments, the parenchymal cells include beta cells.

In some embodiments the first population of cells and/or the second population of cells are induced pluripotent (iPSC)-derived cells, engineered cells, primary cells, embryonic stem cells (ESC)-derived cells, or transdifferentiated cells. In some embodiments, the primary cells include primary cells expanded in vitro.

In some embodiments, the engineered cells are engineered to express or secrete a protein (e.g., an antibody, a cytokine, an enzyme, a coagulation factor, or a hormone). In some embodiments, the protein is an endogenous human protein or an engineered protein.

In some embodiments, the first and/or second population of cells includes endocrine, exocrine, paracrine, heterocrine, autocrine, or juxtacrine cells.

In some embodiments, the first and/or second population of cells includes leading cells, adrenal cortical cells, pituitary cells, thyrocytes, granulosa cells, mammary gland epithelial cells, thymocytes, thymic epithelial cells, hypothalamus cells, skeletal muscle cells, smooth muscle cells, and/or neuronal cells.

In some embodiments, the pituitary cells include thyrotropic pituitary cells, lactotropic pituitary cells, corticotropic pituitary cells, somatotropic pituitary cells, and/or gonadotropic pituitary cells. In some embodiments, the neuronal cells include dopaminergic cells.

In some embodiments, the first and/or second population of cells includes parenchymal cells (e.g., hepatocytes, pancreatic exocrine cells, myocytes, pancreatic endocrine cells, neurons, enterocytes, adipocytes, splenic cells, kidney cells, biliary cells, Kupffer cells, stellate cells, cardiac muscle cells, alveolar cells, bronchiolar cells, club cells, urothelial cells, mucous cells, parietal cells, chief cells, G cells, goblet cells, enteroendocrine cells, Paneth cells, M cells, tuft cells, glial cells, gall bladder cells, keratinocytes, melanocytes, Merkel cells, Langerhans cells, osteocytes, osteoclasts, esophageal cells, photoreceptor cells, and corneal epithelial cells). In some embodiments, the parenchymal cells are pancreatic cells (e.g., alpha, beta, gamma, delta, epsilon cells, or any combination thereof). In some embodiments, the parenchymal cells include beta cells.

In some embodiments, the first and/or second population of cells are engineered cells, primary cells, or transdifferentiated cells.

In some embodiments, the method includes encapsulating two or more populations of cells (e.g., two, three, four, five, six, seven, eight, nine, ten, or more populations of cells).

In some embodiments, a method described herein may include providing a population of stromal cells.

In some embodiments, the ratio of parenchymal cells to stromal cells is from 1:10 to 4:1 (e.g., 1:10 to 4:1, 1:10 to 3:1, 1:10 to 2:1, 1:10 to 1:1, 1:9 to 4:1, 1:9 to 3:1, 1:9 to 2:1, 1:9 to 1:1, 1:8 to 4:1, 1:8 to 3:1, 1:8 to 2:1, 1:8 to 1:1, 1:7 to 4:1, 1:7 to 3:1, 1:7 to 2:1, 1:7 to 1:1, 1:6 to 4:1, 1:6 to 3:1, 1:6 to 2:1, 1:6 to 1:1, 1:5 to 4:1, 1:5 to 3:1, 1:5 to 2:1, 1:5 to 1:1, 1:4 to 4:1, 1:4 to 3:1, 1:4 to 2:1, 1:4 to 1:1, 1:3 to 4:1, 1:3 to 3:1, 1:3 to 2:1, 1:3 to 1:1, 1:2 to 4:1, 1:2 to 3:1, 1:2 to 2:1, 1:2 to 1:1, 1:1 to 4:1, 1:1 to 3:1, 1:1 to 2:1, or 1:0 to 1:1).

For example, in some embodiments, the ratio of parenchymal cells to stromal cells is from 1:9 to 4:1. In some embodiments, the ratio of parenchymal cells to stromal cells is from 1:8 to 4:1. In some embodiments, the ratio of parenchymal cells to stromal cells is from 1:7 to 4:1. In some embodiments, the ratio of parenchymal cells to stromal cells is from 1:6 to 4:1. In some embodiments, the ratio of parenchymal cells to stromal cells is from 1:5 to 4:1. In some embodiments, the ratio of parenchymal cells to stromal cells is from 1:4 to 4:1. In some embodiments, the ratio of parenchymal cells to stromal cells is from 1:3 to 4:1. In some embodiments, the ratio of parenchymal cells to stromal cells is from 1:2 to 4:1. In some embodiments, the ratio of parenchymal cells to stromal cells is from 1:1 to 4:1. In some embodiments, the ratio of parenchymal cells to stromal cells is from 1:0 to 4:1.

In some embodiments, the ratio of parenchymal cells to stromal cells is from 1:10 to 3:1. In some embodiments, the ratio of parenchymal cells to stromal cells is from 1:9 to 3:1. In some embodiments, the ratio of parenchymal cells to stromal cells is from 1:8 to 3:1. In some embodiments, the ratio of parenchymal cells to stromal cells is from 1:7 to 3:1. In some embodiments, the ratio of parenchymal cells to stromal cells is from 1:6 to 3:1. In some embodiments, the ratio of parenchymal cells to stromal cells is from 1:5 to 3:1. In some embodiments, the ratio of parenchymal cells to stromal cells is from 1:4 to 3:1. In some embodiments, the ratio of parenchymal cells to stromal cells is from 1:3 to 3:1. In some embodiments, the ratio of parenchymal cells to stromal cells is from 1:2 to 3:1. In some embodiments, the ratio of parenchymal cells to stromal cells is from 1:1 to 3:1. In some embodiments, the ratio of parenchymal cells to stromal cells is from 1:0 to 3:1.

In some embodiments, the ratio of parenchymal cells to stromal cells is from 1:10 to 2:1. In some embodiments, the ratio of parenchymal cells to stromal cells is from 1:9 to 2:1. In some embodiments, the ratio of parenchymal cells to stromal cells is from 1:8 to 2:1. In some embodiments, the ratio of parenchymal cells to stromal cells is from 1:7 to 2:1. In some embodiments, the ratio of parenchymal cells to stromal cells is from 1:6 to 2:1. In some embodiments, the ratio of parenchymal cells to stromal cells is from 1:5 to 2:1. In some embodiments, the ratio of parenchymal cells to stromal cells is from 1:4 to 2:1. In some embodiments, the ratio of parenchymal cells to stromal cells is from 1:3 to 2:1. In some embodiments, the ratio of parenchymal cells to stromal cells is from 1:2 to 2:1. In some embodiments, the ratio of parenchymal cells to stromal cells is from 1:1 to 2:1. In some embodiments, the ratio of parenchymal cells to stromal cells is from 1:0 to 2:1.

In some embodiments, the ratio of parenchymal cells to stromal cells is from 1:10 to 1:1. In some embodiments, the ratio of parenchymal cells to stromal cells is from 1:9 to 1:1. In some embodiments, the ratio of parenchymal cells to stromal cells is from 1:8 to 1:1. In some embodiments, the ratio of parenchymal cells to stromal cells is from 1:7 to 1:1. In some embodiments, the ratio of parenchymal cells to stromal cells is from 1:6 to 1:1. In some embodiments, the ratio of parenchymal cells to stromal cells is from 1:5 to 1:1. In some embodiments, the ratio of parenchymal cells to stromal cells is from 1:4 to 1:1. In some embodiments, the ratio of parenchymal cells to stromal cells is from 1:3 to 1:1. In some embodiments, the ratio of parenchymal cells to stromal cells is from 1:2 to 1:1. In some embodiments, the ratio of parenchymal cells to stromal cells is from 1:1 to 1:0.

Using any of the methods described herein, the disclosure provides a composition including a population of aggregates that includes stromal cells and parenchymal cells produced by said methods.

### Parenchymal Cells

A device described herein can be used to aggregate one or more populations of cells, one of which may include parenchymal cells (e.g., hepatocytes, pancreatic exocrine cells, myocytes, pancreatic endocrine cells, neurons, enterocytes, adipocytes, splenic cells, kidney cells, biliary cells, Kupffer cells, stellate cells, cardiac muscle cells, alveolar cells, bronchiolar cells, club cells, urothelial cells, mucous cells, parietal cells, chief cells, G cells, goblet cells, enteroendocrine cells, Paneth cells, M cells, tuft cells, glial cells, gall bladder cells, keratinocytes, melanocytes, Merkel cells, Langerhans cells, osteocytes, osteoclasts, esophageal cells, photoreceptor cells, and corneal epithelial cells). For example, in some embodiments, the parenchymal cells are hepatocytes. In some embodiments, the parenchymal cells are pancreatic exocrine cells. In some embodiments, the parenchymal cells are myocytes. In some embodiments, the parenchymal cells are pancreatic endocrine cells. In some embodiments, the parenchymal cells are neurons. In some embodiments, the parenchymal cells are enterocytes. In some embodiments, the parenchymal cells are adipocytes. In some embodiments, the parenchymal cells are splenic cells. In some embodiments, the parenchymal cells are kidney cells. In some embodiments, the parenchymal cells are biliary cells. In some embodiments, the parenchymal cells are Kupffer cells. In some embodiments, the parenchymal cells are stellate cells. In some embodiments, the parenchymal cells are cardiac muscle cells. In some embodiments, the parenchymal cells are alveolar cells. In some embodiments, the parenchymal cells are bronchiolar cells. In some embodiments, the parenchymal cells are club cells. In some embodiments, the parenchymal cells are urothelial cells. In some embodiments, the parenchymal cells are mucous cells. In some embodiments, the parenchymal cells are parietal cells. In some embodiments, the parenchymal cells are chief cells. In some embodiments, the parenchymal cells are G cells. In some embodiments, the parenchymal cells are goblet cells. In some embodiments, the parenchymal cells are enteroendocrine cells. In some embodiments, the parenchymal cells are Paneth cells. In some embodiments, the parenchymal cells are M cells. In some embodiments, the parenchymal cells are tuft cells. In some embodiments, the parenchymal cells are glial cells. In some embodiments, the parenchymal cells are gall bladder cells. In some embodiments, the parenchymal cells are keratinocytes. In some embodiments, the parenchymal cells are melanocytes. In some embodiments, the parenchymal cells are Merkel cells. In some embodiments, the parenchymal cells are Langerhans cells. In some embodiments, the parenchymal cells are osteocytes. In some embodiments, the parenchymal cells are osteoclasts. In some embodiments, the parenchymal cells are esophageal cells. In some embodiments, the parenchymal cells are photoreceptor cells. In some embodiments, the parenchymal cells are corneal epithelial cells.

In some embodiments, the parenchymal cells are pancreatic cells (e.g., alpha, beta, gamma, delta, epsilon cells, or any combination thereof). In some embodiments, the parenchymal cells are alpha cells. In some embodiments, the parenchymal cells are beta cells. In some embodiments, the parenchymal cells are gamma cells. In some embodiments, the parenchymal cells are delta cells. In some embodiments, the parenchymal cells are epsilon cells.

In some embodiments, the parenchymal cells are hepatocytes (e.g., primary human hepatocytes (PHH)).

In some embodiments, the ratio of hepatocytes to stromal cells is from 1:10 to 4:1 (e.g., 1:10 to 4:1, 1:10 to 3:1, 1:10 to 2:1, 1:10 to 1:1, 1:9 to 4:1, 1:9 to 3:1, 1:9 to 2:1, 1:9 to 1:1, 1:8 to 4:1, 1:8 to 3:1, 1:8 to 2:1, 1:8 to 1:1, 1:7 to 4:1, 1:7 to 3:1, 1:7 to 2:1, 1:7 to 1:1, 1:6 to 4:1, 1:6 to 3:1, 1:6 to 2:1, 1:6 to 1:1, 1:5 to 4:1, 1:5 to 3:1, 1:5 to 2:1, 1:5 to 1:1, 1:4 to 4:1, 1:4 to 3:1, 1:4 to 2:1, 1:4 to 1:1, 1:3 to 4:1, 1:3 to 3:1, 1:3 to 2:1, 1:3 to 1:1, 1:2 to 4:1, 1:2 to 3:1, 1:2 to 2:1, 1:2 to 1:1, 1:1 to 4:1, 1:1 to 3:1, 1:1 to 2:1, or 1:0 to 1:1).

For example, in some embodiments, the ratio of hepatocytes to stromal cells is from 1:9 to 4:1. In some embodiments, the ratio of hepatocytes to stromal cells is from 1:8 to 4:1. In some embodiments, the ratio of hepatocytes to stromal cells is from 1:7 to 4:1. In some embodiments, the ratio of hepatocytes to stromal cells is from 1:6 to 4:1. In some embodiments, the ratio of hepatocytes to stromal cells is from 1:5 to 4:1. In some embodiments, the ratio of hepatocytes to stromal cells is from 1:4 to 4:1. In some embodiments, the ratio of hepatocytes to stromal cells is from 1:3 to 4:1. In some embodiments, the ratio of hepatocytes to stromal cells is from 1:2 to 4:1. In some embodiments, the ratio of hepatocytes to stromal cells is from 1:1 to 4:1. In some embodiments, the ratio of hepatocytes to stromal cells is from 1:0 to 4:1.

In some embodiments, the ratio of hepatocytes to stromal cells is from 1:10 to 3:1. In some embodiments, the ratio of hepatocytes to stromal cells is from 1:9 to 3:1. In some embodiments, the ratio of hepatocytes to stromal cells is from 1:8 to 3:1. In some embodiments, the ratio of hepatocytes to stromal cells is from 1:7 to 3:1. In some embodiments, the ratio of hepatocytes to stromal cells is from 1:6 to 3:1. In some embodiments, the ratio of hepatocytes to stromal cells is from 1:5 to 3:1. In some embodiments, the ratio of hepatocytes to stromal cells is from 1:4 to 3:1. In some embodiments, the ratio of hepatocytes to stromal cells is from 1:3 to 3:1. In some embodiments, the ratio of hepatocytes to stromal cells is from 1:2 to 3:1. In some embodiments, the ratio of hepatocytes to stromal cells is from 1:1 to 3:1. In some embodiments, the ratio of hepatocytes to stromal cells is from 1:0 to 3:1.

In some embodiments, the ratio of hepatocytes to stromal cells is from 1:10 to 2:1. In some embodiments, the ratio of hepatocytes to stromal cells is from 1:9 to 2:1. In some embodiments, the ratio of hepatocytes to stromal cells is from 1:8 to 2:1. In some embodiments, the ratio of hepatocytes to stromal cells is from 1:7 to 2:1. In some embodiments, the ratio of hepatocytes to stromal cells is from 1:6 to 2:1. In some embodiments, the ratio of hepatocytes to stromal cells is from 1:5 to 2:1. In some embodiments, the ratio of hepatocytes to stromal cells is from 1:4 to 2:1. In some embodiments, the ratio of hepatocytes to stromal cells is from 1:3 to 2:1. In some embodiments, the ratio of hepatocytes to stromal cells is from 1:2 to 2:1. In some embodiments, the ratio of hepatocytes to stromal cells is from 1:1 to 2:1. In some embodiments, the ratio of hepatocytes to stromal cells is from 1:0 to 2:1.

In some embodiments, the ratio of hepatocytes to stromal cells is from 1:10 to 1:1. In some embodiments, the ratio of hepatocytes to stromal cells is from 1:9 to 1:1. In some embodiments, the ratio of hepatocytes to stromal cells is from 1:8 to 1:1. In some embodiments, the ratio of hepatocytes to stromal cells is from 1:7 to 1:1. In some embodiments, the ratio of hepatocytes to stromal cells is from 1:6 to 1:1. In some embodiments, the ratio of hepatocytes to stromal cells is from 1:5 to 1:1. In some embodiments, the ratio of hepatocytes to stromal cells is from 1:4 to 1:1. In some embodiments, the ratio of hepatocytes to stromal cells is from 1:3 to 1:1. In some embodiments, the ratio of hepatocytes to stromal cells is from 1:2 to 1:1. In some embodiments, the ratio of hepatocytes to stromal cells is from 1:1 to 1:0.

In some embodiments, the parenchymal cells are PHH.

In some embodiments, the population of PHH includes an amount of from 2.5×10⁴ to 1.8×10¹¹ (e.g., from 3×10⁴ to 1.8×10¹¹, 4×10⁴ to 1.8×10¹¹, 5×10⁴ to 1.8×10¹¹, 1×10⁵ to 1.8×10¹¹, 4×10⁵ to 1.8×10¹¹, 5×10⁵ to 1.8×10¹¹, 6×10⁵ to 1.8×10¹¹, 7×10⁵ to 1.8×10¹¹, 8×10⁵ to 1.8×10¹¹, 9×10⁵ to 1.8×10¹¹, 1×10⁶ to 1.8×10¹¹, 2×10⁶ to 1.8×10¹¹, 3×10⁶ to 1.8×10¹¹, 4×10⁶ to 1.8×10¹¹, 5×10⁶ to 1.8×10¹¹, 6×10⁶ to 1.8×10¹¹, 7×10⁶ to 1.8×10¹¹, 8×10⁶ to 1.8×10¹¹, 9×10⁶ to 1.8×10¹¹, 1×10⁷ to 1.8×10¹¹, 2×10⁷ to 1.8×10¹¹, 1.8×10⁷ to 1.8×10¹¹, 4×10⁷ to 1.8×10¹¹, 5×10⁷ to 1.8×10¹¹, 6×10⁷ to 1.8×10¹¹, 7×10⁷ to 1.8×10¹¹, 8×10⁷ to 1.8×10¹¹, 9×10⁷ to 1.8×10¹¹, 1×10⁸ to 1.8×10¹¹, 2×10⁸ to 1.8×10¹¹, 3×10⁸ to 1.8×10¹¹, 4×10⁸ to 1.8×10¹¹, 5×10⁸ to 1.8×10¹¹, 6×10⁸ to 1.8×10¹¹, 7×10⁸ to 1.8×10¹¹, 8×10⁸ to 1.8×10¹¹, 9×10⁸ to 1.8×10¹¹, 1×10⁹ to 1.8×10¹¹, 2×10⁹ to 1.8×10¹¹, 3×10⁹ to 1.8×10¹¹, 4×10⁹ to 1.8×10¹¹, 5×10⁹ to 1.8×10¹¹, 6×10⁹ to 1.8×10¹¹, 7×10⁹ to 1.8×10¹¹, 8×10⁹ to 1.8×10¹¹, 9×10⁹ to 1.8×10¹¹, 1×10¹⁰ to 1.8×10¹¹, 2×10¹⁰ to 1.8×10¹¹, 3×10¹⁰ to 1.8×10¹¹, 4×10¹⁰ to 1.8×10¹¹, 5×10¹⁰ to 1.8×10¹¹, 6×10¹⁰ to 1.8×10¹¹, 7×10¹⁰ to 1.8×10¹¹, 8×10¹⁰ to 1.8×10¹¹, 9×10¹⁰ to 1.8×10¹¹, or 1×10¹¹ to 1.8×10¹¹) PHH. For example, in some embodiments, the population of PHH includes an amount of from 3×10⁴ to 1.8×10¹¹. In some embodiments, the population of PHH includes an amount of from 4×10⁴ to 1.8×10¹¹. In some embodiments, the population of PHH includes an amount of from 5×10⁴ to 1.8×10¹¹. In some embodiments, the population of PHH includes an amount of from 1×10⁵ to 1.8×10¹¹. In some embodiments, the population of PHH includes an amount of from 2×10⁵ to 1.8×10¹¹. In some embodiments, the population of PHH includes an amount of from 3×10⁵ to 1.8×10¹¹ PHH. In some embodiments, the population of PHH includes an amount of from 4×10⁵ to 1.8×10¹¹ PHH. In some embodiments, the population of PHH includes an amount of from 5×10⁵ to 1.8×10¹¹ PHH. In some embodiments, the population of PHH includes an amount of from 6×10⁵ to 1.8×10¹¹ PHH. In some embodiments, the population of PHH includes an amount of from 7×10⁵ to 1.8×10¹¹ PHH. In some embodiments, the population of PHH includes an amount of from 8×10⁵ to 1.8×10¹¹ PHH. In some embodiments, the population of PHH includes an amount of from 9×10⁵ to 1.8×10¹¹ PHH. In some embodiments, the population of PHH includes an amount of from 1×10⁶ to 1.8×10¹¹ PHH. In some embodiments, the population of PHH includes an amount of from 2×10⁶ to 1.8×10¹¹ PHH. In some embodiments, the population of PHH includes an amount of from 3×10⁶ to 1.8×10¹¹ PHH. In some embodiments, the population of PHH includes an amount of from 4×10⁶ to 1.8×10¹¹ PHH. In some embodiments, the population of PHH includes an amount of from 5×10⁶ to 1.8×10¹¹ PHH. In some embodiments, the population of PHH includes an amount of from 6×10⁶ to 1.8×10¹¹ PHH. In some embodiments, the population of PHH includes an amount of from 7×10⁶ to 1.8×10¹¹ PHH. In some embodiments, the population of PHH includes an amount of from 8×10⁶ to 1.8×10¹¹ PHH. In some embodiments, the population of PHH includes an amount of from 9×10⁶ to 1.8×10¹¹. In some embodiments, the population of PHH includes an amount of from 1×10⁷ to 1.8×10¹¹ PHH. In some embodiments, the population of PHH includes an amount of from 2×10⁷ to 1.8×10¹¹ PHH. In some embodiments, the population of PHH includes an amount of from 3×10⁷ to 1.8×10¹¹ PHH. In some embodiments, the population of PHH includes an amount of from 4×10⁷ to 1.8×10¹¹ PHH. In some embodiments, the population of PHH includes an amount of from 5×10⁷ to 1.8×10¹¹ PHH. In some embodiments, the population of PHH includes an amount of from 6×10⁷ to 1.8×10¹¹ PHH. In some embodiments, the population of PHH includes an amount of from 7×10⁷ to 1.8×10¹¹ PHH. In some embodiments, the population of PHH includes an amount of from 8×10⁷ to 1.8×10¹¹ PHH. In some embodiments, the population of PHH includes an amount of from 9×10⁷ to 1.8×10¹¹ PHH. In some embodiments, the population of PHH includes an amount of from 1×10⁸ to 1.8×10¹¹ PHH. In some embodiments, the population of PHH includes an amount of from 2×10⁸ to 1.8×10¹¹ PHH. In some embodiments, the population of PHH includes an amount of from 3×10⁸ to 1.8×10¹¹ PHH. In some embodiments, the population of PHH includes an amount of from 4×10⁸ to 1.8×10¹¹ PHH. In some embodiments, the population of PHH includes an amount of from 5×10⁸ to 1.8×10¹¹ PHH. In some embodiments, the population of PHH includes an amount of from 6×10⁸ to 1.8×10¹¹ PHH. In some embodiments, the population of PHH includes an amount of from 7×10⁸ to 1.8×10¹¹ PHH. In some embodiments, the population of PHH includes an amount of from 8×10⁸ to 1.8×10¹¹ PHH. In some embodiments, the population of PHH includes an amount of from 9×10⁸ to 1.8×10¹¹ PHH. In some embodiments, the population of PHH includes an amount of from 1×10⁹ to 1.8×10¹¹ PHH. In some embodiments, the population of PHH includes an amount of from 2×10⁹ to 1.8×10¹¹ PHH. In some embodiments, the population of PHH includes an amount of from 3×10⁹ to 1.8×10¹¹ PHH. In some embodiments, the population of PHH includes an amount of from 4×10⁹ to 1.8×10¹¹ PHH. In some embodiments, the population of PHH includes an amount of from 5×10⁹ to 1.8×10¹¹ PHH. In some embodiments, the population of PHH includes an amount of from 6×10⁹ to 1.8×10¹¹ PHH. In some embodiments, the population of PHH includes an amount of from 7×10⁹ to 1.8×10¹¹ PHH. In some embodiments, the population of PHH includes an amount of from 8×10⁹ to 1.8×10¹¹ PHH. In some embodiments, the population of PHH includes an amount of from 9×10⁹ to 1.8×10¹¹ PHH. In some embodiments, the population of PHH includes an amount of from 1×10¹⁰ to 1.8×10¹¹ PHH. In some embodiments, the population of PHH includes an amount of from 2×10¹⁰ to 1.8×10¹¹ PHH. In some embodiments, the population of PHH includes an amount of from 3×10¹⁰ to 1.8×10¹¹ PHH. In some embodiments, the population of PHH includes an amount of from 4×10¹⁰ to 1.8×10¹¹ PHH. In some embodiments, the population of PHH includes an amount of from 5×10¹⁰ to 1.8×10¹¹ PHH. In some embodiments, the population of PHH includes an amount of from 6×10¹⁰ to 1.8×10¹¹ PHH. In some embodiments, the population of PHH includes an amount of from 7×10¹⁰ to 1.8×10¹¹ PHH. In some embodiments, the population of PHH includes an amount of from 8×10¹⁰ to 1.8×10¹¹ PHH. In some embodiments, the population of PHH includes an amount of from 9×10¹⁰ to 1.8×10¹¹ PHH. In some embodiments, the population of PHH includes an amount of from 1×10¹¹ to 1.8×10¹¹ PHH.

### Stromal Cells

A device described herein can be used to aggregate one or more populations of cells, one of which may include parenchymal cells stromal cells (e.g., fibroblasts, endothelial cells, or pericytes). For example, in some embodiments, the stromal cells are fibroblasts (e.g., normal human dermal fibroblasts or neonatal foreskin fibroblasts). In some embodiments, the stromal cells are endothelial cells. In some embodiments, the stromal cells are pericytes. In some embodiments, the stromal cells are normal human dermal fibroblasts. In some embodiments, the stromal cells are neonatal foreskin fibroblasts.

In some embodiments, the stromal cells are normal human dermal fibroblasts.

In some embodiments, the population of stromal cells (e.g., fibroblasts) is up to 1.8×10¹² (e.g., from 1×10³ to 1.8×10¹², from 2×10³ to 1.8×10¹², from 3×10³ to 1.8×10¹², from 4×10³ to 1.8×10¹², from 5×10³ to 1.8×10¹², from 6×10³ to 1.8×10¹², from 7×10³ to 1.8×10¹², from 8×10³ to 1.8×10¹², from 9×10³ to 1.8×10¹², from 1×10⁴ to 1.8×10¹², from 2×10⁴ to 1.8×10¹², from 3×10⁴ to 1.8×10¹², from 4×10⁴ to 1.8×10¹², from 5×10⁴ to 1.8×10¹², from 6×10⁴ to 1.8×10¹², from 7×10⁴ to 1.8×10¹², from 8×10⁴ to 1.8×10¹², from 9×10⁴ to 1.8×10¹², from 1×10⁵ to 1.8×10¹², from 2×10⁵ to 1.8×10¹², from 3×10⁵ to 1.8×10¹², from 4×10⁵ to 1.8×10¹², from 5×10⁵ to 1.8×10¹², from 6×10⁵ to 1.8×10¹², from 7×10⁵ to 1.8×10¹², from 8×10⁵ to 1.8×10¹², from 9×10⁵ to 1.8×10¹², from 1×10⁶ to 1.8×10¹² from 2×10⁶ to 1.8×10¹², 3×10⁶ to 1.8×10¹², 4×10⁶ to 1.8×10¹², 5×10⁶ to 1.8×10¹², 6×10⁶ to 1.8×10¹², 7×10⁶ to 1.8×10¹², 8×10⁶ to 1.8×10¹², 9×10⁶ to 1.8×10¹², from 1×10⁷ to 1.8×10¹², from 2×10⁷ to 1.8×10¹², from 18×10⁷ to 1.8×10¹², from 4×10⁷ to 1.8×10¹², from 5×10⁷ to 1.8×10¹², from 6×10⁷ to 1.8×10¹², from 7×10⁷ to 1.8×10¹², from 8×10⁷ to 1.8×10¹², from 9×10⁷ to 1.8×10¹², from 1×10⁸ to 1.8×10¹², from 2×10⁸ to 1.8×10¹², from 3×10⁸ to 1.8×10¹², from 4×10⁸ to 1.8×10¹², from 5×10⁸ to 1.8×10¹², from 6×10⁸ to 1.8×10¹², from 7×10⁸ to 1.8×10¹², from 8×10⁸ to 1.8×10¹², from 9×10⁸ to 1.8×10¹², from 1×10⁹ to 1.8×10¹², from 2×10⁹ to 1.8×10¹², from 3×10⁹ to 1.8×10¹², from 4×10⁹ to 1.8×10¹², from 5×10⁹ to 1.8×10¹², from 6×10⁹ to 1.8×10¹², from 7×10⁹ to 1.8×10¹², from 8×10⁹ to 1.8×10¹², from 9×10⁹ to 1.8×10¹², from 1×10¹⁰ to 1.8×10¹², from 2×10¹⁰ to 1.8×10¹², from 3×10¹⁰ to 1.8×10¹², from 4×10¹⁰ to 1.8×10¹², from 5×10¹⁰ to 1.8×10¹², from 6×10¹⁰ to 1.8×10¹², from 7×10¹⁰ to 1.8×10¹², from 8×10¹⁰ to 1.8×10¹², from 9×10¹⁰ to 1.8×10¹², from 1×10¹¹ to 1.8×10¹², from 2×10¹¹ to 1.8×10¹², from 3×10¹¹ to 1.8×10¹², from 4×10¹¹ to 1.8×10¹², from 5×10¹¹ to 1.8×10¹², from 6×10¹¹ to 1.8×10¹², from 7×10¹¹ to 1.8×10¹², from 8×10¹¹ to 1.8×10¹², from 9×10¹¹ to 1.8×10¹², or from 1×10¹² to 1.8×10¹²) stromal cells. For example, in some embodiments, the population of stromal cells includes an amount of from 1 to 1.8×10¹² stromal cells. In some embodiments, the population of stromal cells includes an amount of from 10 to 1.8×10¹² stromal cells. In some embodiments, the population of stromal cells includes an amount of from 100 to 1.8×10¹² stromal cells. In some embodiments, the population of stromal cells includes an amount of from 1×10³ to 1.8×10¹² stromal cells. In some embodiments, the population of stromal cells includes an amount from 2×10³ to 1.8×10¹² stromal cells. In some embodiments, the population of stromal cells includes an amount of from 3×10³ to 1.8×10¹² stromal cells. In some embodiments, the population of stromal cells includes an amount of from 4×10³ to 1.8×10¹² stromal cells. In some embodiments, the population of stromal cells includes an amount of from 5×10³ to 1.8×10¹² stromal cells. In some embodiments, the population of stromal cells includes an amount of from 6×10³ to 1.8×10¹² stromal cells. In some embodiments, the population of stromal cells includes an amount of from 7×10³ to 1.8×10¹² stromal cells. In some embodiments, the population of stromal cells includes an amount of from 8×10³ to 1.8×10¹² stromal cells. In some embodiments, the population of stromal cells includes an amount of from 9×10³ to 1.8×10¹² stromal cells. In some embodiments, the population of stromal cells includes an amount of from 1×10⁴ to 1.8×10¹² stromal cells. In some embodiments, the population of stromal cells includes an amount of from 2×10⁴ to 1.8×10¹² stromal cells. In some embodiments, the population of stromal cells includes an amount of from 3×10⁴ to 1.8×10¹² stromal cells. In some embodiments, the population of stromal cells includes an amount of from 4×10⁴ to 1.8×10¹² stromal cells. In some embodiments, the population of stromal cells includes an amount of from 5×10⁴ to 1.8×10¹² stromal cells. In some embodiments, the population of stromal cells includes an amount of from 6×10⁴ to 1.8×10¹² stromal cells. In some embodiments, the population of stromal cells includes an amount of from 7×10⁴ to 1.8×10¹² stromal cells. In some embodiments, the population of stromal cells includes an amount of from 8×10⁴ to 1.8×10¹² stromal cells. In some embodiments, the population of stromal cells includes an amount of from 9×10⁴ to 1.8×10¹² stromal cells. In some embodiments, the population of stromal cells includes an amount of from 1×10⁵ to 1.8×10¹² stromal cells. In some embodiments, the population of stromal cells includes an amount of from 2×10⁵ to 1.8×10¹² stromal cells. In some embodiments, the population of stromal cells includes an amount of from 3×10⁵ to 1.8×10¹² stromal cells. In some embodiments, the population of stromal cells includes an amount of from 4×10⁵ to 1.8×10¹² stromal cells. In some embodiments, the population of stromal cells includes an amount of from 5×10⁵ to 1.8×10¹² stromal cells. In some embodiments, the population of stromal cells includes an amount of from 6×10⁵ to 1.8×10¹² stromal cells. In some embodiments, the population of stromal cells includes an amount of from 7×10⁵ to 1.8×10¹² stromal cells. In some embodiments, the population of stromal cells includes an amount of from 8×10⁵ to 1.8×10¹² stromal cells. In some embodiments, the population of stromal cells includes an amount of from 9×10⁵ to 1.8×10¹² stromal cells. In some embodiments, the population of stromal cells includes an amount of from 1×10⁶ to 1.8×10¹² stromal cells. In some embodiments, the population of stromal cells includes an amount of from 2×10⁶ to 1.8×10¹² stromal cells. In some embodiments, the population of stromal cells includes an amount of from 3×10⁶ to 1.8×10¹² stromal cells. In some embodiments, the population of stromal cells includes an amount of from 4×10⁶ to 1.8×10¹² stromal cells. In some embodiments, the population of stromal cells includes an amount of from 5×10⁶ to 1.8×10¹² stromal cells. In some embodiments, the population of stromal cells includes an amount of from 6×10⁶ to 1.8×10¹² stromal cells. In some embodiments, the population of stromal cells includes an amount of from 7×10⁶ to 1.8×10¹² stromal cells. In some embodiments, the population of stromal cells includes an amount of from 8×10⁶ to 1.8×10¹² stromal cells. In some embodiments, the population of stromal cells includes an amount of from 9×10⁶ to 1.8×10¹² stromal cells. In some embodiments, the population of stromal cells includes an amount of from 1×10⁷ to 1.8×10¹² stromal cells. In some embodiments, the population of stromal cells includes an amount of from 2×10⁷ to 1.8×10¹² stromal cells. In some embodiments, the population of stromal cells includes an amount of from 3×10⁷ to 1.8×10¹² stromal cells. In some embodiments, the population of stromal cells includes an amount of from 4×10⁷ to 1.8×10¹² stromal cells. In some embodiments, the population of stromal cells includes an amount of from 5×10⁷ to 1.8×10¹² stromal cells. In some embodiments, the population of stromal cells includes an amount of from 6×10⁷ to 1.8×10¹² stromal cells. In some embodiments, the population of stromal cells includes an amount of from 7×10⁷ to 1.8×10¹² stromal cells. In some embodiments, the population of stromal cells includes an amount of from 8×10⁷ to 1.8×10¹² stromal cells. In some embodiments, the population of stromal cells includes an amount of from 9×10⁷ to 1.8×10¹² stromal cells. In some embodiments, the population of stromal cells includes an amount of from 1×10⁸ to 1.8×10¹² stromal cells. In some embodiments, the population of stromal cells includes an amount of from 2×10⁸ to 1.8×10¹² stromal cells. In some embodiments, the population of stromal cells includes an amount of from 3×10⁸ to 1.8×10¹² stromal cells. In some embodiments, the population of stromal cells includes an amount of from 4×10⁸ to 1.8×10¹² stromal cells. In some embodiments, the population of stromal cells includes an amount of from 5×10⁸ to 1.8×10¹² stromal cells. In some embodiments, the population of stromal cells includes an amount of from 6×10⁸ to 1.8×10¹² stromal cells. In some embodiments, the population of stromal cells includes an amount of from 7×10⁸ to 1.8×10¹² stromal cells. In some embodiments, the population of stromal cells includes an amount of from 8×10⁸ to 1.8×10¹² stromal cells. In some embodiments, the population of stromal cells includes an amount of from 9×10⁸ to 1.8×10¹² stromal cells. In some embodiments, the population of stromal cells includes an amount of from 1×10⁹ to 1.8×10¹² stromal cells. In some embodiments, the population of stromal cells includes an amount of from 2×10⁹ to 1.8×10¹² stromal cells. In some embodiments, the population of stromal cells includes an amount of from 3×10⁹ to 1.8×10¹² stromal cells. In some embodiments, the population of stromal cells includes an amount of from 4×10⁹ to 1.8×10¹² stromal cells. In some embodiments, the population of stromal cells includes an amount of from 5×10⁹ to 1.8×10¹² stromal cells. In some embodiments, the population of stromal cells includes an amount of from 6×10⁹ to 1.8×10¹² stromal cells. In some embodiments, the population of stromal cells includes an amount of from 7×10⁹ to 1.8×10¹² stromal cells. In some embodiments, the population of stromal cells includes an amount of from 8×10⁹ to 1.8×10¹² stromal cells. In some embodiments, the population of stromal cells includes an amount of from 9×10⁹ to 1.8×10¹² stromal cells. In some embodiments, the population of stromal cells includes an amount of from 1×10¹⁰ to 1.8×10¹² stromal cells. In some embodiments, the population of stromal cells includes an amount of from 2×10¹⁰ to 1.8×10¹² stromal cells. In some embodiments, the population of stromal cells includes an amount of from 3×10¹⁰ to 1.8×10¹² stromal cells. In some embodiments, the population of stromal cells includes an amount of from 4×10¹⁰ to 1.8×10¹² stromal cells. In some embodiments, the population of stromal cells includes an amount of from 5×10¹⁰ to 1.8×10¹² stromal cells. In some embodiments, the population of stromal cells includes an amount of from 6×10¹⁰ to 1.8×10¹² stromal cells. In some embodiments, the population of stromal cells includes an amount of from 7×10¹⁰ to 1.8×10¹² stromal cells. In some embodiments, the population of stromal cells includes an amount of from 8×10¹⁰ to 1.8×10¹² stromal cells. In some embodiments, the population of stromal cells includes an amount of from 9×10¹⁰ to 1.8×10¹² stromal cells. In some embodiments, the population of stromal cells includes an amount of from 1×10¹¹ to 1.8×10¹² stromal cells. In some embodiments, the population of stromal cells includes an amount of from 2×10¹¹ to 1.8×10¹² stromal cells. In some embodiments, the population of stromal cells includes an amount of from 3×10¹¹ to 1.8×10¹² stromal cells. In some embodiments, the population of stromal cells includes an amount of from 4×10¹¹ to 1.8×10¹² stromal cells. In some embodiments, the population of stromal cells includes an amount of from 5×10¹¹ to 1.8×10¹² stromal cells. In some embodiments, the population of stromal cells includes an amount of from 6×10¹¹ to 1.8×10¹² stromal cells. In some embodiments, the population of stromal cells includes an amount of from 7×10¹¹ to 1.8×10¹² stromal cells. In some embodiments, the population of stromal cells includes an amount of from 8×10¹¹ to 1.8×10¹² stromal cells. In some embodiments, the population of stromal cells includes an amount of from 9×10¹¹ to 1.8×10¹² stromal cells. In some embodiments, the population of stromal cells includes an amount of from 1×10¹² to 1.8×10¹² stromal cells.

### EXAMPLES

The following examples are put forth so as to provide those of ordinary skill in the art with a description of how the compositions and methods described herein may be used and evaluated and are intended to be purely exemplary of the invention and are not intended to limit the scope of what the inventors regard as their invention.

### Example 1. Use of Device for Cell Aggregation

A device as described herein containing a plate (1) with a microwell film composite (2) and macroscopic manifold (3) is provided (FIG. 6). The device may contain an upper manifold (9), retaining ring (10), and a basin (16). The microwell film composite includes microwells (4) and a permeable body (5) disposed below the microwells that includes pores (6) (FIG. 12).

A medium may be introduced into port (7) to provide fluid communication throughout the plate. A population of stromal cells and a population of parenchymal cells may be provided through port (7) into the device to direct the medium that includes the cells into the microwells in the microwell film composite (2). Additional media is introduced into port (7) to allow the cells to settle into microwells (4). The macroscopic manifold (3) applies a positive pressure to direct flow throughout the device.

The introduction of stromal cells and parenchymal cells can be sequential, e.g., the stromal cells may be introduced, e.g., in a first medium, and the parenchymal cells may be introduced, e.g., in a second medium. Similarly, the parenchymal cells may be introduced, e.g., in a first medium, and the stromal cells may be introduced, e.g., in a second medium. Alternatively, the stromal cells and the parenchymal cells may be introduced in the same medium. Once the cells are in the microwells (4), an aggregation medium may be introduced into port (7) and flown into the microwells (4) (FIG. 10). The cells may then be cultured to form aggregates. Once the aggregates form, a negative pressure may be applied to port (7) in the macroscopic manifold (3) to reverse the flow (FIG. 11). The aggregates may be released from the microwells an collected through a port.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

Other embodiments are within the claims.

1. A device comprising one or more plates comprising a microwell film composite comprising a first layer comprising a plurality of microwells and a second layer comprising a permeable body, wherein the permeable body comprises a plurality of pores, and wherein the device comprises one or more ports for fluid input and/or output.

2. The device of claim 1, further comprising a macroscopic manifold, wherein the macroscopic manifold is connected to the microwell film composite and is configured to direct fluid flow to the microwell film composite.

3. The device of claim 2, wherein the macroscopic manifold comprises one or more of the ports for fluid input and/or output.

4. The device of claim 2 or 3, wherein the device further comprises one or more gaskets, wherein the one or more gaskets form a seal between the macroscopic manifold and the microwell film composite.

5. The device of any one of claims 2-4, wherein the macroscopic manifold further comprises one or more seal ports in fluid connection with the microwell film composite, wherein the one or more seal ports are configured to direct fluid flow and/or pressure to or from the microwell film composite.

6. The device of claim 5, wherein the macroscopic manifold further comprises one or more foil seals attached to the seal ports.

7. The device of claim 6, wherein the macroscopic manifold further comprises one or more seal punches positioned in the one or more seal ports, wherein the one or more seal punches are configured to break the one or more foil seals.

8. The device of claim 7, wherein the macroscopic manifold comprises two or more seal ports and two or more seal punches.

9. The device of claim 8, wherein the macroscopic manifold comprises three or more seal ports and three or more seal punches.

10. The device of claim 9, wherein the macroscopic manifold comprises four or more seal ports and four or more seal punches.

11. The device of any one of claims 7-10, wherein the macroscopic manifold further comprises one or more seal bellows that connect the one or more seal punches to the one or more seal ports.

12. The device of any one of claims 7-11, wherein the device further comprises a cam bar connected to the one or more seal punches.

13. The device of claim 12, wherein the device further comprises an actuator connected to the cam bar and configured to engage the one or more seal punches.

14. The device of claim 12 or 13, wherein the cam bar is connected to the macroscopic manifold.

15. The device of any one of claims 5-14, wherein the macroscopic manifold further comprises a sealed outlet in fluid connection with the one or more seal ports, wherein the sealed outlet is configured to direct fluid flow from the microwells to the one or more seal ports.

16. The device of any one of claims 5-15, wherein the macroscopic manifold further comprises a sealed inlet in fluid connection with the one or more seal ports, wherein the sealed inlet is configured to direct fluid flow from the microwells to the one or more seal ports.

17. The device of any one of claims 1-16, further comprising a collection outlet in fluid communication with the first layer comprising the plurality of microwells.

18. The device of any one of claims 1-17, wherein the plate has a surface area of from 100 mm² to 1×10⁵ mm².

19. The device of any one of claims 1-18, wherein the plate has a density of microwells of from 1 well/mm² to 10⁶ wells/mm².

20. The device of any one of claims 1-19, wherein each microwell has a diameter of from 1 μm to 1 mm.

21. The device of any one of claims 1-20, wherein the permeable body comprises a polymer or stainless steel.

22. The device of claim 21, wherein the polymer comprises polycarbonate, polyester, polystyrene, polytetrafluoroethylene (PTFE), collagen-coated PTFE, polyethylene terephthalate (PET), polysulfone (PES), nylon, or cellulose acetate.

23. The device of any one of claims 1-22, wherein the microwells comprise a stainless material or a polymer.

24. The device of claim 23, wherein the microwells comprise a polymer.

25. The device of claim 24, wherein the polymer comprises polystyrene, PET, polycarbonate, polypropylene, cellulose acetate, PES, or fluorinated ethylene propylene (FEP).

26. The device of any one of claims 1-25, wherein each pore has a diameter of from 0.1 μm to 15 μm.

27. The device of claim 26, wherein each pore has a diameter of from 0.1 μm to 3 μm.

28. The device of any one of claims 1-27, wherein the permeable body has a density of pores across the permeable body of from 1×10⁴ pores/cm² to 1×10⁹ pores/cm².

29. The device of any one of claims 1-28, wherein the permeable body is a filter.

30. The device of any one of claims 1-29, wherein the permeable body comprises a plurality of non-woven non-linear pathways.

31. The device of any one of claims 1-30, wherein each of the pores is cylindrical.

32. The device of any one of claims 1-31, wherein the layer comprising microwells further comprises a passivation coating.

33. The device of claim 32, wherein the passivation coating comprises a non-covalent coating and/or a covalent coating.

34. The device of claim 32 or 33, wherein the non-covalent coating comprises a block co-polymer, polyethylene glycol (PEG), streptavidin, albumin/biotin, phospholipid surfactants, hyaluronic acid, or poly-lysine-based adherence.

35. The device of any one of claims 32-34, wherein the covalent coating comprises a covalent attachment.

36. The device of claim 35, wherein the covalent attachment comprises carboxylic acid/amine bonds, disulfide bonds, n-hydroxysuccinimide (NHS) esters, NHS, maleimide, cycloadditions, epoxy, amine, carboxy, aldehyde, p-phenylene diisothiocyanate (PDITC), maleimide, thiol, poly-l-lysine, streptavidin, or neutravidin.

37. The device of any one of claims 1-36, wherein the microwells are conical, spherical, cylindrical, pyramidal, or chaliced shaped.

38. The device of any one of claims 1-37, wherein the one or more plates further comprise an upper manifold, wherein the upper manifold is in fluid connection with the macroscopic manifold and is configured to direct fluid flow and/or fluid distribution to or from the macroscopic manifold to the microwell film composite.

39. The device of claim 38, wherein the one or more plates further comprise a retaining ring positioned between the upper manifold and the microwell film composite, wherein the retaining ring is configured to position the upper manifold from 10 μm to 100 mm from the microwell film composite.

40. The device of any one of claims 37-39, wherein the upper manifold comprises an upper surface and a lower surface, and the one or more plates further comprise a plate cover positioned on the upper surface of the upper manifold.

41. The device of any one of claims 1-40, wherein the first layer and the second layer are components of a laminate.

42. The device of claim 41, wherein the laminate further comprises a third layer comprising a perforation film, wherein the third layer is adjoined to the layer comprising the permeable body and is configured to direct fluid pressure to the microwell film composite.

43. The device of claim 42, wherein the perforation film comprises polystyrene.

44. The device of claim 42 or 43, wherein the microwells comprise polystyrene, wherein the permeable body comprises PTFE or collagen-coated PTFE, and wherein the perforation film comprises polystyrene.

45. The device of any one of claims 1-44, wherein the one or more plates further comprise a film superstructure, wherein the film superstructure is connected to the microwell film composite and is configured to provide structural integrity to the layers of the microwell film composite.

46. The device of claim 45, wherein the film superstructure comprises one or more gaps across the film superstructure, wherein the one or more gaps allow fluid to flow through the layer comprising a permeable body.

47. The device of claim 46, wherein the one or more gaps are configured to be a distance of 10 μm to 100 mm apart.

48. The device of any one of claims 45-47, wherein the one or more plates further comprise a membrane film standoff, wherein the membrane film standoff is connected to the film superstructure to provide structural integrity to the microwell film composite.

49. The device of any one of claims 45-48, wherein the one or more plates further comprise a basin, wherein the basin is connected to the film superstructure to provide a receptacle for fluid that flows through the layer comprising the permeable body.

50. The device of claim 49, wherein the basin has a height of from 10 μm to 100 mm.

51. The device of any one of claims 1-50, wherein the device comprises five or more plates.

52. The device of claim 51, wherein the device comprises ten or more plates.

53. The device of claim 52, wherein the device comprises fifteen or more plates.

54. The device of claim 53, wherein the device comprises twenty or more plates.

55. A method of aggregating a first population of cells and a second population of cells, comprising:
(a) providing the device of any one of claims 1-54 and a perfusion medium comprising a first population of cells and a second population of cells; and
(b) introducing the perfusion medium comprising the first population of cells and the second population of cells into the device through one of the ports to direct the perfusion medium comprising the first population of cells and the second population of cells into the microwells.

56. The method of claim 55, wherein the device comprises a macroscopic manifold connected to the microwell film composite and step (b) comprises applying a positive pressure to the macroscopic manifold to direct fluid flow to the microwell film composite.

57. The method of claim 55 or 56, wherein step (b) comprises shaking or centrifuging the device.

58. The method of any one of claims 55-57, wherein each pore allows flow of the aggregation medium, and wherein each pore prevents flow of the first population of cells and the second population of cells.

59. The method of any one of claims 55-58, wherein step (b) further comprises introducing an aggregation medium through one of the ports.

60. The method of claim 59, wherein step (b) comprises applying a pressure to the macroscopic manifold to direct the aggregation medium into the microwells.

61. The method of any one of claims 55-60, further comprising (c) culturing the first population of cells and the second population of cells in the microwells under conditions such that aggregates of the first population of cells and the second population cells form in the microwells.

62. The method of any one of claims 59-61, wherein step (c) further comprises culturing the first population of cells and the second population of cells in the microwells under conditions in the aggregation medium such that aggregates of the first population of cells and the second population cells form in the microwells.

63. The method of any one of claims 59-62, further comprising washing or removing the aggregation medium.

64. The method of claim 63, further comprising (d) releasing the aggregates from the microwells, thereby producing a population of aggregates comprising stromal cells and parenchymal cells.

65. The method of claim 64, wherein step (d) comprises applying a negative pressure to the macroscopic manifold to release the aggregates.

66. The method of any one of claims 55-65, further comprising collecting the population of aggregates.

67. The method of claim 66, wherein the device comprises a collection outlet in fluid communication with the first layer comprising the plurality of microwells, and the method comprises collecting the population of aggregates as it passes through the collection outlet.

68. The method of any one of claims 55-67, wherein the first population of cells and/or the second population of cells are induced pluripotent (iPSC)-derived cells, engineered cells, primary cells, embryonic stem cells (ESC)-derived cells, or transdifferentiated cells.

69. The method of any one of claims 55-68, wherein the first population of cells comprises stromal cells and the second population of cells comprises parenchymal cells.

70. The method of claim 69, wherein the parenchymal cells hepatocytes, pancreatic exocrine cells, myocytes, pancreatic endocrine cells, neurons, enterocytes, adipocytes, splenic cells, kidney cells, biliary cells, Kupffer cells, stellate cells, cardiac muscle cells, alveolar cells, bronchiolar cells, club cells, urothelial cells, mucous cells, parietal cells, chief cells, G cells, goblet cells, enteroendocrine cells, Paneth cells, M cells, tuft cells, glial cells, gall bladder cells, keratinocytes, melanocytes, Merkel cells, Langerhans cells, osteocytes, osteoclasts, esophageal cells, photoreceptor cells, or corneal epithelial cells.

71. The method of claim 70, wherein the hepatocytes are primary human hepatocytes (PHH), iPSC-derived hepatocytes, or ESC-derived hepatocytes.

72. The method of claim 71, wherein the population of PHH comprises from 2.5×10⁴ to 1.8×10¹¹ PHH.

73. The method of any one of claims 70-72, wherein a ratio of hepatocytes to stromal cells is from 1:10 to 4:1.

74. The method of any one of claims 69-73, wherein the stromal cells are fibroblasts, endothelial cells, or pericytes.

75. The method of claim 74, wherein the fibroblasts are normal human dermal fibroblasts or neonatal foreskin fibroblasts.

76. The method of claim 75, wherein the fibroblasts are normal human dermal fibroblasts.

77. The method of any one of claims 69-76, wherein the population of stromal cells comprises from 6×10³ to 1.8×10¹² stromal cells.

78. The method of any one of claims 69-77, wherein the population of parenchymal cells and the population of stromal cells are provided in a ratio of parenchymal cells to stromal cells of from 1:10 to 4:1.

79. The method of any one of claims 69-78, wherein the one or more plates further comprise an upper manifold, wherein the upper manifold is in fluid connection with the macroscopic manifold and directs fluid flow and/or fluid distribution to or from the macroscopic manifold to the microwell film composite.

80. The method of any one of claims 69-79, wherein the macroscopic manifold further comprises one or more seal ports in fluid connection with the microwell film composite, wherein the one or more seal ports direct fluid flow and/or pressure to or from the microwell film composite.

81. The method of claim 80, wherein the macroscopic manifold further comprises one or more foil seals attached to the seal ports.

82. The method of claim 81, wherein the macroscopic manifold further comprises one or more seal punches positioned in the one or more seal ports, and the seal punches break the one or more foil seals.

83. The method of claim 82, wherein the device further comprises a cam bar connected to the one or more seal punches, and the method comprises actuating the cam bar to engage the one or more seal punches to break the one or more foil seals.

84. A population of aggregates comprising a first population of cells and a second population of cells produced by the method of any one of claims 53-83.

85. The population of aggregates of claim 84, wherein the first population of cells comprises stromal cells and the second population of cells comprises parenchymal cells.

86. The population of aggregates of claim 85, wherein the first population of cells comprises fibroblasts and the second population of cells comprises hepatocytes.
